(19) United States
(12) Patent Application Publication
(10) Pub. No.: US 2023/0125841 A1
(43) Pub. Date: Apr. 27, 2023

(54) PLASMA SYSTEM WITH DIRECTIONAL FEATURES

(71) Applicant: CAPS Medical Ltd., Netanya (IL)

(72) Inventor: Ilan Oleg UCHITEL, Kfar-Saba (IL)

(73) Assignee: CAPS Medical Ltd., Netanya (IL)

(21) Appl. No.: 17/912,587

(22) PCT Filed: Mar. 18, 2021
(86) PCT No.: PCT/IL2021/050302

§ 371 Date: Sep. 19, 2022

Related U.S. Application Data
(60) Provisional application No. 62/991,642, filed on Mar. 19, 2020.

(51) Int. Cl. A61N 1/44; H05H 1/24

(52) U.S. Cl. CPC A61N 1/44; H05H 1/2431; H05H 2245/32

(57) ABSTRACT

Adjustable distal tips of cold plasma generating devices configured for introduction to and operation within narrow intra-body confines. In some embodiments, a plasma delivery tip of a cold plasma generating device is expandable from a compact delivery configuration, allowing device operation with plasma plume parameters difficult to achieve within size constraints of a narrow delivery catheter and/or endoscope working channel. Additionally or alternatively, in some embodiments, operating parameters of a plasma delivery tip are adjustable to tune characteristics of the plasma plume. Adjustable parameters optionally include, for example: lumen diameter, lumen aperture shape/direction, discharge electrode geometry, dielectric barrier characteristics, and/or relative placement of these components, including placement relative to a stream of ionizing gas. In some embodiments, plasma delivery tip elements are adapted to assist device navigation and/or tissue penetration.

### RELATED APPLICATIONS

This application claims the benefit of priority under 35 USC § 119(e) of U.S. Provisional Patent Application No. 62/991,642 filed Mar. 19, 2020, the contents of which are incorporated herein by reference in their entirety.

This application is one of four co-filed applications including PCT application having Attorney Docket Nos. 85937, 85988, and 85989, the contents of each of which is included herein by reference in its entirety.

### FIELD AND BACKGROUND OF THE INVENTION

The present invention, in some embodiments thereof, relates to the field of cold atmospheric plasma generation and more particularly, to delivery of cold plasma within body cavities.

Plasma is a general term encompassing compositions of ionized gas, generally including free electrons and ions, as well as neutral atoms and molecules, and often free radicals. Plasma may be produced by electric discharge through gas, causing gas atoms or molecules to be excited and ionize. During the past decade, significant interest in plasma applications has grown. Some applications are based on Dielectric Barrier Discharge (DBD) for generation of the non-thermal plasma of low temperature, or so-called “cold” plasma. Such cold plasma is a low-ionized and non-thermal plasma generated at atmospheric pressure conditions. It has been found that cold plasma can be used for various applications in medicine and industry.

### SUMMARY OF THE INVENTION

According to an aspect of some embodiments of the present disclosure, there is provided a plasma delivery tip of a medical-grade plasma generating device, including: a gas delivery lumen having a proximal-to-distal axis, and through which a flow of ionization gas flows to a distal aperture of the gas delivery lumen; a discharge electrode, which transmits a high voltage to the flow of ionization gas when attached to a high voltage source; and a dielectric barrier layer positioned between the discharge electrode and the flow of ionization gas, along which cold plasma is generated by dielectric barrier discharge when the discharge electrodes transmits the high voltage; wherein the plasma delivery tip is dynamically adjustable in geometry to modify a parameter affecting the generation of plasma.

According to some embodiments of the present disclosure, the plasma is dynamically adjustable by modifying a relative position of at least two of the gas delivery lumen, the discharge electrode, and the dielectric barrier layer.

According to some embodiments of the present disclosure, the relative position is adjusted by moving the discharge electrode along the proximal-to-distal axis, relative to the gas delivery lumen.

According to some embodiments of the present disclosure, the relative position is adjusted by offsetting the discharge electrode radially within the gas delivery lumen.

According to some embodiments of the present disclosure, the relative position is maintained by a positioning support positioned within the gas delivery lumen.

According to some embodiments of the present disclosure, the relative position is adjustable by rotating the positioning support.

According to some embodiments of the present disclosure, the relative position is adjustable by sliding the positioning support.

According to some embodiments of the present disclosure, the plasma delivery tip sized for insertion to a target region through an aperture or conduit 7 mm in diameter or less.

According to some embodiments of the present disclosure, the plasma is dynamically adjustable by modifying a shape of at least one of the gas delivery lumen, the discharge electrode, and the dielectric barrier layer.

According to some embodiments of the present disclosure, the adjusted shape includes a changed diameter of the gas delivery lumen.

According to some embodiments of the present disclosure, adjustment of the diameter of the gas delivery lumen is actuated by advancing the gas delivery lumen from confinement within a sheath and allowing elasticity of the gas delivery lumen to expand it to a width greater than the sheath along at least one axis.

According to some embodiments of the present disclosure, adjustment of the diameter of the gas delivery lumen is actuated by forces exerted longitudinally along the proximal-to-distal axis.

According to some embodiments of the present disclosure, adjustment of the diameter of the gas delivery lumen includes expansion of the lumen by release of longitudinal compression along the proximal-to-distal axis.

According to some embodiments of the present disclosure, adjustment of the diameter of the gas delivery lumen includes expansion of the lumen by release of longitudinal stretching along the proximal-to-distal axis.

According to some embodiments of the present disclosure, there is provided a sheath layer circumferentially surrounding at least a portion of the dielectric barrier layer, and attached to the dielectric barrier layer on a distal side; wherein the dielectric barrier layer defines the gas delivery lumen, and a diameter of the gas delivery lumen is adjusted by adjusting relative forces exerted along the proximal-distal axis on the sheath layer and the dielectric barrier layer from a proximal side of the plasma delivery tip.

According to some embodiments of the present disclosure, adjustment of the diameter of the gas delivery lumen is actuated by forces acting circumferentially around the proximal-to-distal axis.

According to some embodiments of the present disclosure, the discharge electrode includes a wire extending around at least 75% of a circumference of the dielectric barrier layer, the dielectric barrier layer defines the gas delivery lumen, and tightening the wire shrinks the dielectric barrier layer, reducing a diameter of the gas delivery lumen.

According to some embodiments of the present disclosure, the discharge electrode includes a conductive element extending at least 75% of a circumference of the dielectric barrier layer, and adapted to expand or shrink according to an increase or decrease in diameter of the dielectric barrier layer.

According to some embodiments of the present disclosure, the conductive element includes a conductive material deposited onto an elastic supporting substrate.

According to some embodiments of the present disclosure, the conductive element includes a ring with a gap that expands or shrinks according to an increase or decrease in diameter of the dielectric barrier layer.

According to some embodiments of the present disclosure, the adjusted shape includes a changed outer diameter of the gas delivery lumen.

According to some embodiments of the present disclosure, the adjusted shape includes a shape of the discharge electrode.

According to some embodiments of the present disclosure, the shape of the discharge electrode adjusts to a changed length along the proximal-to-distal axis.

According to some embodiments of the present disclosure, the shape of the discharge electrode adjusts to a changed diameter of the discharge electrode.

According to some embodiments of the present disclosure, the dielectric barrier includes a plurality of circumferentially arranged segments, adapted to expand by flaring radially outward.

According to some embodiments of the present disclosure, the discharge electrode includes a plurality of circumferentially distributed segments, each extending along the proximal-to-distal axis.

According to some embodiments of the present disclosure, the discharge electrode includes a shape memory alloy that changes shape upon heating to a predetermined temperature.

According to some embodiments of the present disclosure, the discharge electrode changes to a shape which generates less plasma upon heating to the predetermined temperature.

According to some embodiments of the present disclosure, the discharge electrode circumferentially surrounds at least 75% of the dielectric barrier layer, and the dielectric barrier layer circumferentially surrounds the gas delivery lumen.

According to some embodiments of the present disclosure, a distal tip of the gas delivery lumen is beveled to form a pointed tip.

According to some embodiments of the present disclosure, the plasma delivery tip includes channels extending along the proximal-to-distal axis configured to return the ionization gas in a proximal direction.

According to some embodiments of the present disclosure, the channels comprise a plurality of circumferentially separated projections separated by indentations around an outer surface of the plasma delivery tip.

According to some embodiments of the present disclosure, the channels are helical.

According to some embodiments of the present disclosure, the dielectric barrier layer circumferentially surrounds the discharge electrode, and the gas delivery lumen circumferentially surrounds the dielectric barrier layer.

According to an aspect of some embodiments of the present disclosure, there is provided a method of configuring a plume of cold plasma delivered from a medical-grade plasma delivery tip, the method including: flowing an ionization gas through a gas delivery lumen, and alongside a discharge electrode separated from the ionization gas by a dielectric barrier layer; supplying high voltage electrical pulses to the discharge electrode; and adjusting at least one of the gas delivery lumen, the discharge electrode, and the dielectric barrier layer to re-configure plasma generating parameters of the plasma delivery tip.

According to some embodiments of the present disclosure, the plasma delivery tip is positioned on a distal side of a probe conduit, and the adjusting includes actuating the change in shape from a control positioned on a proximal side of the probe conduit.

According to an aspect of some embodiments of the present disclosure, there is provided a method of configuring a plume of cold plasma delivered from a medical-grade plasma delivery tip, the method including: advancing a plasma delivery tip distally in a collapsed configuration until it protrudes from a distal end of a sheath; and expanding the plasma delivery tip.

According to some embodiments of the present disclosure, the expanding includes expanding a gas delivery lumen of the plasma delivery tip.

According to some embodiments of the present disclosure, the expanding includes expanding a discharge electrode of the plasma delivery tip.

According to some embodiments of the present disclosure, the expanding includes increasing a thickness of a dielectric barrier layer of the plasma delivery tip.

According to some embodiments of the present disclosure, the collapsed configuration of the plasma delivery tip has an outer diameter of 7 mm or less.

According to some embodiments of the present disclosure, the sheath is flexible.

According to some embodiments of the present disclosure, the sheath is rigid.

According to an aspect of some embodiments of the present disclosure, there is provided a plasma delivery tip of a medical-grade plasma generating device, including: a gas delivery lumen having a proximal-to-distal axis, and along which a flow of ionization gas flows to a distal aperture of the gas delivery lumen; and a discharge electrode, which, when attached to a high voltage source, transmits a high voltage to generate plasma within the flow of ionization gas; and a control operable to adjust cold plasma production by modifying at least one of: a shape of at least one of the gas delivery lumen and the discharge electrode, and a relative position of the gas delivery lumen and the discharge electrode.

According to an aspect of some embodiments of the present disclosure, there is provided a method of configuring a plume of cold plasma delivered from a medical-grade plasma delivery tip, the method including: flowing an ionization gas through a gas delivery lumen, to impinge on a discharge electrode; supplying high voltage electrical pulses to the discharge electrode; and adjusting, during the supplying, the relative positioning of the gas delivery lumen and the discharge electrode to adjust the generation of cold plasma.

According to some embodiments of the present disclosure, the adjusting includes re-orienting the discharge electrode with respect to the flow of ionization gas.

According to some embodiments of the present disclosure, the adjusting is performed for a portion of the discharge electrode positioned outside of the gas delivery lumen.

According to some embodiments of the present disclosure, the adjusting is performed by moving the portion of the discharge electrode to a position offset from a longitudinal axis of a distal end of the gas delivery lumen.

According to an aspect of some embodiments of the present disclosure, there is provided a method of configuring a plume of cold plasma delivered from a medical-grade plasma delivery tip, the method including: flowing an ionization gas through a distal portion of gas delivery lumen, to impinge on a portion of a discharge electrode; and supplying high voltage electrical pulses to the discharge electrode; wherein the distal portion of the gas delivery lumen has a central longitudinal axis, and the portion of the discharge electrode is positioned away from the longitudinal axis, at a distance beyond a lumenal cross-section radius of the gas delivery lumen.

According to an aspect of some embodiments of the present disclosure, there is provided a method of delivering plasma to a target surface, including: positioning a distal end of a working channel within a lumen including the target surface; advancing a plasma delivery tip out of the working channel along a proximal-to-distal axis of the working channel; and moving the plasma delivery tip relative to the working channel while generating at least one plasma plume oriented to a direction oblique or perpendicular to the proximal-to-distal axis.

According to some embodiments of the present disclosure, the moving includes bending the plasma delivery tip.

According to some embodiments of the present disclosure, the moving includes rotating the plasma delivery tip.

According to some embodiments of the present disclosure, the method includes generating a plurality of plasma plumes oriented to a same direction oblique or perpendicular to the proximal-to-distal axis.

According to some embodiments of the present disclosure, the method includes generating a plurality of plasma plumes oriented to a plurality of radial directions oblique to the proximal-to-distal axis.

According to some embodiments of the present disclosure, the advancing releases the plasma delivery tip out of a confining lumen; a portion of the plasma delivery tip re-orients relative to the proximal-to-distal axis as it is released from the confining lumen; and the plasma plume is generated from a flow of ionization gas exiting an aperture of the re-orienting portion of the plasma delivery tip.

According to some embodiments of the present disclosure, the confining lumen includes the working channel.

According to some embodiments of the present disclosure, the confining lumen includes a sleeve held at least partially within the working channel.

According to some embodiments of the present disclosure, the moving comprise rotating the plasma delivery tip, and the rotating is performed with a plasma plume generated by a plasma generation site of the plasma delivery tip and oriented to a first angle relative to the proximal-to-distal axis; and then with the plasma plume generated by the plasma generation site oriented to a second angle relative to the proximal-distal axis.

According to some embodiments of the present disclosure, the plasma plume is re-oriented between the first and second angles by a change in bending of the portion of the plasma delivery tip as it is released from the confining lumen.

According to some embodiments of the present disclosure, the portion of the plasma tip includes an elastic tube held straight while in the confining lumen, and predisposed to bend as it is released from the confining lumen.

According to an aspect of some embodiments of the present disclosure, there is provided a plasma delivery tip of a medical-grade plasma generating device, including: a gas delivery lumen having a proximal-to-distal axis, and along which a flow of ionization gas flows to a distal aperture of the gas delivery lumen; a discharge electrode, which transmits a high voltage to the flow of ionization gas when attached to a high voltage source; and an electrical power conduit configured to interconnect the discharge electrode with the high voltage source; wherein the electrical power conduit is also adapted to receive mechanical tension to adjust the plasma delivery tip.

According to some embodiments of the present disclosure, the mechanical tension adjusts a steering angle of the plasma delivery tip.

According to some embodiments of the present disclosure, the plasma delivery tip sized for insertion to a target region through an aperture or conduit 7 mm in diameter or less.

According to an aspect of some embodiments of the present disclosure, there is provided a method of adjusting a plasma plume from a plasma delivery tip of a medical-grade plasma delivery device, the method including: generating a plasma plume including ionization gas ionized by a discharge electrode positioned with the plasma delivery tip and extending from an aperture of the plasma delivery tip; and adjusting an orientation of the aperture by operation of a control member that flexes the plasma delivery tip.

According to some embodiments of the present disclosure, the control member flexes the plasma delivery tip while the plasma delivery tip remains confined within a sheath.

According to some embodiments of the present disclosure, the control member flexes 15 mm or less of the plasma delivery tip.

According to some embodiments of the present disclosure, the control member flexes the plasma delivery tip by rotation of the plasma delivery tip within a sheath.

According to an aspect of some embodiments of the present disclosure, there is provided a plasma delivery tip of a medical-grade plasma generating device, including: a gas delivery lumen having a proximal-to-distal axis, and along which a flow of ionization gas flows distally to an exit aperture of the gas delivery lumen; and a discharge electrode, which transmits a high voltage to the flow of ionization gas when attached to a high voltage source to generate a flow of cold plasma; wherein the exit aperture of the gas delivery lumen is oriented to direct a plasma plume exiting the gas delivery lumen away from the proximal-to-distal axis.

According to some embodiments of the present disclosure, the plasma delivery tip includes a dielectric barrier layer positioned between the discharge electrode and the flow of ionization gas, along which the flow of cold plasma is generated by dielectric barrier discharge when the discharge electrodes transmits the high voltage.

According to some embodiments of the present disclosure, the plasma delivery tip sized for insertion to a target region through an aperture or conduit 7 mm in diameter or less.

According to an aspect of some embodiments of the present disclosure, there is provided a plasma delivery tip of a medical-grade plasma generating device, including: a gas delivery lumen having a proximal-to-distal axis, and along which a flow of ionization gas flows to a distal aperture of the gas delivery lumen; a discharge electrode positioned to ionize the flow of ionization gas into a plasma; and at least one gas return channel extending along the gas delivery lumen, through which the ionization gas returns proximally after exiting the gas delivery lumen.

According to some embodiments of the present disclosure, the at least one gas return channel extends helically around the gas delivery lumen.

According to some embodiments of the present disclosure, the gas return channel is provided with a connector to allow attachment to a source of negative pressure.

According to some embodiments of the present disclosure, the gas return channel is open to a pressure lower than a pressure developed negative pressure.

According to some embodiments of the present disclosure, the plasma is thermally non-damaging.

According to an aspect of some embodiments of the present disclosure, there is provided a method of operating a plasma generating device including: generating a plasma plume that exits a distal end of a lumen of the plasma generating device; and inserting a medical tool along the lumen until it exits the distal end.

According to some embodiments of the present disclosure, the method includes withdrawing an element used in generating the plasma plume from the lumen before inserting the medical tool.

According to some embodiments of the present disclosure, the element includes a discharge electrode.

According to some embodiments of the present disclosure, the element includes a surface that shapes and/or directs the plasma plume.

According to an aspect of some embodiments of the present disclosure, there is provided a plasma delivery tip of a medical-grade plasma generating device, including: a gas delivery lumen having a proximal-to-distal axis, through which a flow of ionization gas flows out of an aperture of the gas delivery lumen; and a discharge electrode, which transmits pulses of high voltage into the flow of ionization gas when attached to a high voltage source via an electrical power conduit, ionizing the ionization gas to a plasma; wherein the electrical power conduit slides distally from within the gas delivery lumen to advance the discharge electrode and act as a guidewire for guiding advance of the gas delivery lumen.

According to some embodiments of the present disclosure, the discharge electrode is encapsulated within a sharp-pointed cap.

According to some embodiments of the present disclosure, the electrical power conduit and discharge electrode are configured to be withdrawn from the gas delivery lumen, allowing the gas delivery lumen to serve as a working channel for delivery of another tool to a distal end of the gas delivery lumen.

According to some embodiments of the present disclosure, the plasma is a thermally non-damaging plasma.

According to some embodiments of the present disclosure, the plasma delivery tip sized for insertion to a target region through an aperture or conduit 7 mm in diameter or less.

According to an aspect of some embodiments of the present disclosure, there is provided a medical-grade plasma generating device including: a first conduit through which a flow of ionization gas flows out of an aperture of the first conduit; and a discharge electrode, which transmits pulses of high voltage into the flow of ionization gas when attached to a high voltage source, ionizing the ionization gas to a plasma; a second conduit, through which the discharge electrode is advanced to an intrabody location for generation of plasma using the flow of ionization gas supplied by the first conduit.

According to some embodiments of the present disclosure, the plasma is a thermally non-damaging plasma.

According to some embodiments of the present disclosure, the first and second conduits are sized for insertion to the intrabody location through an aperture or third conduit 7 mm in diameter or less.

According to an aspect of some embodiments of the present disclosure, there is provided a method of constructing a discharge electrode for use with a medical-grade plasma device, the method including: stripping an outer insulating layer from a distal portion of a coaxial cable; replacing a flexible conducting electrical shielding layer from the distal portion of a coaxial cable with a stiffened electrical shielding layer, leaving a portion of a central conductor of the coaxial cable unshielded; and insulating the unshielded portion of the central conductor with a dielectric barrier layer.

According to some embodiments of the present disclosure, the method includes placing an outer insulating layer back over the stiffened electrical shielding layer.

According to some embodiments of the present disclosure, the coaxial cable has an outer diameter of less than 4 mm.

According to an aspect of some embodiments of the present disclosure, there is provided a discharge assembly of a plasma generating device, including: a coaxial cable having an outer insulator, an outer conductor, an inner insulator, and a central conductor; an electrical shielding layer, stiffer than the outer conductor, and extending distally from the outer conductor; and a discharge electrode within a dielectric barrier layer; wherein the discharge electrode includes a portion of the central conductor extending distally beyond a distal end of the electrical shielding layer, and the dielectric barrier layer includes an insulator provided separately from the inner insulator.

According to some embodiments of the present disclosure, the discharge assembly provided together with the plasma generating device, and operable to generate plasma within a lumen of the plasma generating device.

According to an aspect of some embodiments of the present disclosure, there is provided a plasma delivery tip of a medical-grade plasma generating device for the delivery of plasma to a target surface external to the plasma delivery tip, including: a gas delivery lumen having a proximal-to-distal axis, and along which a flow of ionization gas flows to one or more distal apertures of the gas delivery lumen; and a plurality of discharge electrodes, each placed to generate a corresponding respective plasma plume at a respective plasma generating site through which the flow of ionization gas passes.

According to some embodiments of the present disclosure, flow of the ionization gas through the one or more distal apertures directs the plasma plumes to different respective regions of the target surface.

According to some embodiments of the present disclosure, the plasma plumes partially overlap on the way to the target surface.

According to some embodiments of the present disclosure, the one or more distal apertures comprise a plurality of separate apertures from which respective separate plasma plumes are emitted after generation of plasma by respective different discharge electrodes.

According to some embodiments of the present disclosure, the plurality of discharge electrodes comprise electrodes positioned on a circumference of a lumenal wall of the plasma delivery tip within which the ionization gas flows.

According to some embodiments of the present disclosure, the plurality of discharge electrodes comprise electrodes positioned within the flow of ionization gas.

According to some embodiments of the present disclosure, the electrodes positioned within the flow of ionization gas are circumferentially surrounded by the flow of ionization gas.

According to some embodiments of the present disclosure, the electrodes positioned within the flow of ionization gas are also positioned at least partially distal to the distal aperture out of which the ionization gas used to generate the corresponding respective plasma plume flows.

According to some embodiments of the present disclosure, the electrodes positioned within the flow of ionization gas are also positioned external to the distal aperture out of which the ionization gas used to generate the corresponding respective plasma plume flows.

According to some embodiments of the present disclosure, the discharge electrodes are arranged along the proximal-to-distal axis of the gas delivery lumen, and the corresponding respective plasma plumes are directed laterally away from the axis.

According to an aspect of some embodiments of the present disclosure, there is provided a plasma delivery tip including: a gas delivery lumen having a proximal-to-distal axis, and along which a flow of ionization gas flows to a plurality of distal apertures of the gas delivery lumen; at least one discharge electrode placed to generate plasma within the flow of ionization gas; wherein the distal apertures are oriented to direct plasma plumes emitted from the plasma delivery tip away from the proximal-to-distal axis.

According to some embodiments of the present disclosure, a distal portion of the gas delivery lumen is rotatingly coupled to the plasma delivery tip, and the distal apertures are oriented to direct the flow of the ionization gas out of them in a direction generating thrust that rotates the distal portion of the gas lumen and spins the plasma plumes.

According to some embodiments of the present disclosure, the plasma delivery tip includes a discharge electrode positioned proximal to the rotating distal portion of the gas delivery lumen.

According to some embodiments of the present disclosure, the at least one discharge electrode includes a separate respective discharge electrode positioned to generate plasma at each of the plurality of distal apertures.

According to some embodiments of the present disclosure, the plasma delivery tip includes a sliding electrical coupling through which electrical power is conducted to the distal portion of the plasma delivery tip.

According to some embodiments of the present disclosure, the distal apertures direct the plasma plumes in radially opposite directions.

According to some embodiments of the present disclosure, the distal apertures direct the plasma plumes to at least two different angles away from the proximal-to-distal axis.

According to some embodiments of the present disclosure, the plasma delivery tip also includes a distal aperture that directs a plasma plume along the proximal-to-distal axis.

According to some embodiments of the present disclosure, the plasma delivery tip is sized to be delivered along a working channel of an endoscopic device, and rotatable around the proximal-to-distal axis to distribute plasma from the plasma plumes circumferentially.

According to an aspect of some embodiments of the present disclosure, there is provided a plasma delivery tip of a medical-grade plasma generating device for the delivery of plasma to a target surface external to the plasma delivery tip, including: a gas delivery lumen having a proximal-to-distal axis, and along which a flow of ionization gas flows to a plurality of plasma generation sites; each plasma generation site including an outlet aperture for the ionization gas and a discharge electrode operable to generate a plasma plume from the ionization gas; and a confining lumen confining the plasma generation sites in a collapsed configuration; wherein the plasma generation sites deploy to a deployed configuration upon release from the confining lumen, and the deployed configuration redistributes the outlet apertures to a distribution which is larger along at least one axis than the distribution of the outlet apertures in the collapsed configuration.

According to some embodiments of the present disclosure, the deployed configuration distances each of the outlet apertures from each other.

According to some embodiments of the present disclosure, the deployed configuration aligns the outlet apertures along a line.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically represents a plasma treatment device, according to some embodiments of the present disclosure.

FIG. 1B schematically represents a plasma delivery tip configured with an adjustable lumen diameter, according to some embodiments of the present disclosure;

FIG. 1C schematically represents a plasma delivery tip configured with a tension-adjustable lumenal wall thickness, according to some embodiments of the present disclosure;

FIG. 1D schematically represents a plasma delivery tip configured with co-adjustable lumenal wall thickness and lumen diameter, according to some embodiments of the present disclosure;

FIG. 1E schematically represents a plasma delivery tip configured with telescoping-adjustable lumenal wall thickness and lumen diameter, according to some embodiments of the present disclosure;

FIG. 1F schematically represents a plasma delivery tip configured with a twist-adjustable lumen diameter, according to some embodiments of the present disclosure;

FIG. 1G schematically represents a plasma delivery tip configured with a twist-adjustable lumen diameter, according to some embodiments of the present disclosure;

FIG. 1H schematically illustrates, in cross-section, different thermal measuring device configurations for use with a plasma delivery tip, according to some embodiments of the present disclosure;

FIG. 2A schematically represents a plasma delivery tip configured with an adjustable-length plasma discharge electrode, according to some embodiments of the present disclosure;

FIG. 2B schematically represents a plasma delivery tip configured with an adjustable-diameter plasma discharge electrode, according to some embodiments of the present disclosure;

FIG. 2C schematically represents an end-on view of the adjustable-diameter plasma discharge electrode of FIG. 2B, according to some embodiments of the present disclosure;

FIG. 2D schematically represents a plasma delivery tip configured with an adjustable-diameter plasma discharge electrode, according to some embodiments of the present disclosure;

FIG. 2E schematically represents a plasma delivery tip configured with a lasso-type adjustable-diameter plasma discharge electrode, according to some embodiments of the present disclosure;

FIG. 2F schematically represents a plasma delivery tip configured with an open loop-type adjustable-diameter plasma discharge electrode, according to some embodiments of the present disclosure;

FIG. 2G schematically represents a plasma delivery tip configured with a helix-type adjustable-length plasma discharge electrode, according to some embodiments of the present disclosure;

FIGS. 2H-2J schematically represent a plasma delivery tip configured with a segmented expanding distal end, according to some embodiments of the present disclosure;

FIG. 3A schematically represents a plasma delivery tip configured with a steerable end, according to some embodiments of the present disclosure;

FIG. 3B schematically represents a plasma delivery tip configured with an end configured to constrict to a penetrating cone, according to some embodiments of the present disclosure;

FIG. 4 schematically represents a plasma delivery tip configured with a beveled distal end, according to some embodiments of the present disclosure;

FIG. 5A schematically represents a plasma delivery tip configured with a channeled insulating tube, according to some embodiments of the present disclosure;

FIG. 5B schematically represents a plasma delivery tip configured with a helically channeled insulating tube, according to some embodiments of the present disclosure;

FIG. 5C schematically represents an end-on view of a cross section of the channeled insulating tube of FIGS. 5A-5B, according to some embodiments of the present disclosure;

FIG. 5D schematically represents a plasma delivery tip configured with a helically channeled insulating tube, according to some embodiments of the present disclosure;

FIG. 6A schematically illustrates a plasma delivery tip comprising a discharge electrode assembly which is positioned within a lumen of a gas supply tube, according to some embodiments of the present disclosure;

FIG. 6B schematically illustrates position adjustments of the discharge electrode within a plasma delivery tip, according to some embodiments of the present disclosure;

FIGS. 6C-6D schematically illustrate a positioning support configured for use with a plasma delivery tip comprising a discharge electrode assembly which is positioned within a lumen of a gas supply tube, according to some embodiments of the present disclosure;

FIGS. 6E-6G schematically illustrate positioning support allowing both longitudinal′ and radial position adjustment of a plasma delivery tip comprising a discharge electrode assembly which is positioned within a lumen of a gas supply tube, according to some embodiments of the present disclosure;

FIGS. 7A-7D schematically illustrate adjustable discharge electrodes of various discharge electrode assemblies configured to be positioned within a lumen of a gas supply tube, according to some embodiments of the present disclosure;

FIG. 8A schematically illustrates a discharge electrode assembly configured to be positioned within a lumen of a plasma gas supply tube, and comprising a dielectric barrier layer adjustable by inflation, according to some embodiments of the present disclosure;

FIG. 8B schematically illustrates a discharge electrode assembly configured to be positioned within a lumen of a plasma gas supply tube, and comprising a multi-laminar dielectric barrier layer, according to some embodiments of the present disclosure;

FIG. 9 schematically illustrates a discharge electrode assembly and coaxial cable (an instance of a coaxial cable) configured for use as a guidewire for guiding advance of gas supply tube, according to some embodiments of the present disclosure;

FIGS. 10A-10C schematically represent the optional use of alternative tools with a gas supply tube, according to some embodiments of the present disclosure;

FIGS. 11A-11D schematically illustrate different arrangements of lumens for ionizing gas delivery, plasma/ionizing gas removal, and/or current delivery, according to some embodiments of the present disclosure;

FIGS. 12A-12B schematically illustrates construction details of a small-diameter discharge electrode assembly, according to some embodiments of the present disclosure;

FIGS. 13A-13F schematically illustrate plasma delivery tips configured to generate oblique- and/or perpendicular-angle plasma plumes relative to a longitudinal axis of the plasma delivery tip, according to some embodiments of the present disclosure;

FIG. 14A schematically illustrates scanning delivery of cold plasma to a body lumen, according to some embodiments of the present disclosure;

FIG. 14B schematically illustrates a plasma delivery tip configured for angulation scanning from within a sheath, according to some embodiments of the present disclosure;

FIG. 14C schematically illustrates a plasma delivery tip configured for wire-guided scanning by bending of a gas delivery tube, according to some embodiments of the present disclosure;

FIGS. 15A-15C schematically illustrate a plasma delivery tip configured for rotationally actuated scanning of a plasma plume, according to some embodiments of the present disclosure;

FIG. 16 is a schematic flowchart of a method of adjusting a plasma delivery tip, according to some embodiments of the present disclosure;

FIGS. 17A-17D show a plasma delivery tip that deploys its distal aperture to a cross-section wider than the diameter of the sheath from which tip deploys, according to some embodiments of the present disclosure;

FIGS. 18A-18D show other examples of wide cross-section distal apertures, according to some embodiments of the present disclosure;

FIGS. 19A-19F show examples of wide cross-section distal apertures of plasma delivery tip tubes which themselves house discharge electrode assemblies having elongated cross-sections, according to some embodiments of the present disclosure;

FIGS. 20A-20C schematically illustrate a width-expanding discharge electrode assembly for use with a plasma delivery tip, according to some embodiments of the present disclosure;

FIGS. 21A-21B schematically illustrate a different width-expanding discharge electrode assembly for use with a plasma delivery tip, according to some embodiments of the present disclosure;

FIGS. 22A-22C schematically illustrate a flow-spreading electrode assembly for use with a plasma delivery tip, according to some embodiments of the present disclosure;

FIGS. 23A-23B schematically illustrate an off-axis deploying discharge electrode assembly for use with a plasma delivery tip, according to some embodiments of the present disclosure;

FIGS. 24A-24C schematically illustrate an off-axis deploying electrode assembly for use with a plasma delivery tip having an off-axis directed ionization gas exit aperture, according to some embodiments of the present disclosure;

FIG. 25 schematically illustrates an off-axis deploying discharge electrode assembly for use with a plasma delivery tip, according to some embodiments of the present disclosure;

FIG. 26 schematically illustrates a self-expanding discharge electrode assembly for use with a plasma delivery tip, according to some embodiments of the present disclosure;

FIGS. 27A-27B schematically illustrate a self-expanding discharge electrode assembly for use with a plasma delivery tip, according to some embodiments of the present disclosure;

FIGS. 28A-28C schematically illustrate a plasma delivery tip that packages the self-expanding discharge electrode assembly differently than shown in FIGS. 27A-27B, according to some embodiments of the present disclosure;

FIGS. 29A-29B schematically illustrate plasma delivery tips delivered through a working channel within a sleeve, according to some embodiments of the present disclosure;

FIGS. 30A-30B schematically represent patterns of plasma interaction with a surface generated by rotation of a plasma plume around a longitudinal axis offset from and/or oblique a longitudinal axis of the plasma plume itself, according to some embodiments of the present disclosure;

FIGS. 31A-31C schematically represent a self-orienting plasma delivery tip which is actuatable to reorient a plasma outlet aperture through a range of off-axis orientations with respect to a longitudinal axis of the sleeve and/or working channel that delivers it, according to some embodiments of the present disclosure;

FIGS. 32A-32C schematically represent a self-orienting plasma delivery tip which is actuatable to reorient a plasma outlet aperture through a range of off-axis orientations with respect to a longitudinal axis of the sleeve and/or working channel that delivers it, according to some embodiments of the present disclosure;

FIGS. 33A-33C schematically represent a self-orienting plasma delivery tip which is actuatable to reorient a plasma outlet aperture through a range of off-axis orientations with respect to a longitudinal axis of the sleeve and/or working channel that delivers it, according to some embodiments of the present disclosure;

FIGS. 34A-34B schematically illustrate a plasma delivery tip provided with a plurality of discharge electrode assemblies, according to some embodiments of the present disclosure;

FIG. 35 shown the plasma delivery tip of FIGS. 34A-34B operated in a steerable configuration, according to some embodiments of the present disclosure;

FIGS. 36A-36B schematically illustrate a plasma delivery tip provided with a plurality of discharge electrode assemblies operable together with a corresponding plurality of individual gas supply tubes, according to some embodiments of the present disclosure;

FIGS. 37 and 39 schematically illustrate a plasma delivery tip provided with a plurality of discharge electrode assemblies, operable together with a corresponding plurality of individual gas supply tubes which spread to radially expanded shape upon advance from confinement, according to some embodiments of the present disclosure;

FIG. 38 schematically illustrates a plasma delivery tip provided with a plurality of discharge electrode assemblies, operable together with a corresponding plurality of individual gas supply tubes which are linearly arranged, and branch from a common lumen of gas supply tube, according to some embodiments of the present disclosure;

FIGS. 40A-40C schematically illustrate a manifold-type plasma delivery tip, according to some embodiments of the present disclosure;

FIG. 41 schematically illustrates another manifold-type plasma delivery tip, according to some embodiments of the present disclosure;

FIGS. 42-43 schematically additional manifold-type plasma delivery tips, according to some embodiments of the present disclosure;

FIGS. 44, 45A-45B and 46A-46B schematically illustrate embodiments of self-rotating plasma delivery tips, according to some embodiments of the present disclosure;

FIGS. 47A-47C and 48 schematically illustrate alternative embodiments of internal component of self-rotating plasma delivery tips, according to some embodiments of the present disclosure; and

FIGS. 49 and 50 schematically illustrate plasma delivery tips configured with a plurality of longitudinally spaced plasma generation sites, according to some embodiments of the present disclosure.

### DESCRIPTION OF SPECIFIC EMBODIMENTS OF THE INVENTION

The present invention, in some embodiments thereof, relates to the field of cold atmospheric plasma generation and more particularly, to delivery of cold plasma within body cavities.

### Overview

A broad aspect of some embodiments of the present disclosure relates to methods and devices which provide cold (non-thermal) plasma to living tissue under conditions of medically acceptable temperature, safety, and sterility (i.e., the devices are medical-grade plasma generating devices); and in particular at a temperature which remains below a threshold of thermal injury and/or protein denaturation. Herein, medical-grade plasma generating devices which generate plasma below the threshold of outright thermal destruction of cellular structures are also referred to as “thermally non-damaging” medical-grade plasma generating devices.

Thermal coagulation is considered to occur above 60° C. Certain proteins may denature or otherwise be functionally impaired from temperatures well below 60°; e.g., even at mildly hyperthermic (“fever”) temperatures of 40° C. or so. However, temperatures moderately below 60°, for example 50° C., can be applied for limited periods without inducing significant thermal damage (e.g., about 1-2 minutes; potentially longer depending on total thermal energy applied into and rate of heat transfer out of the biological target). Still cooler temperatures (e.g., about 40-45° C.) typically can be applied to a local area for longer periods without inducing thermal damage. Plasmas generated at any of these temperatures may be considered “cold” or “non-thermal”, in the sense of the plasma not being in thermodynamic equilibrium as it is created—electrons in the plasma may have very high-temperature energies (e.g., thousands of degrees Celsius), while the much heavier ions remain cool.

In biological applications, plasma which is “cold” in the equilibrium sense may be nevertheless divided, based on its effects, into hotter plasma (e.g., at or above 60° C.) which produces immediate structural changes in tissue as a result of thermal damage, and cooler plasma (e.g., at or below 50° C.) applied to avoid thermal damage. Primary effects of this cooler plasma type are mediated by chemical reactions induced by the presence of ionic atomic and/or molecular species. Potentially, cold plasma of the thermally non-damaging type has therapeutic effects involving the impairment or modification (as distinct from the outright destruction) of biological pathways. For example, it may act to destroy and/or trigger the destruction of tumor cells and/or pathogens such as viral particles, bacteria, fungi, and/or prions. Being chemical-reactive in nature, this destruction is potentially more selective than thermal destruction, e.g., having differential effects on healthy cells versus abnormal cells and/or invasive pathogens.

A suggested mechanism of therapeutic action generated by these reactions involves sensitivity to free radicals. In some cases there may be a differential sensitivity; i.e., a targeted tumor and/or pathogen is more sensitive to free radicals than nearby healthy tissue. Treatment effects potentially depend on interactions between parameters of the target (e.g., surrounding fluid, target size, and/or target type) and parameters of the plasma delivered (e.g., generated ionized species, their concentrations and/or ratios). Parameters of the plasma delivered in turn potentially are affected by parameters of the generation of the plasma (e.g., ionizing medium composition and/or electrical parameters), and parameters of the plasm plume itself (e.g., geometry, containment, flow, and/or quenching).

Parameters of plasma generation which produce these therapeutic effects potentially vary significantly across different plasma generator designs, ionization gas used as a medium, contents of plasma generated, environment, tumor/pathogen size, and/or tumor/pathogen type. Accordingly, it is a potential advantage for a plasma delivery device to be operable under a range of operating parameters.

Herein, references to “plasma” and “plasma plume” refer more specifically to cold plasma which is also thermally non-damaging; that is, plasma at a temperature of 50° C. or less, preferably delivered at a temperature within or below the range of febrile temperatures (e.g., less than 45° C.), and optionally delivered at temperatures at or even less than normal human body temperature, e.g., within a range of about 20° C.-30° C. The cold plasma is generally delivered under conditions of approximately atmospheric pressure, and accordingly also referred to as “cold atmospheric plasma” or CAP. The plasma plume is generated from a supply of “ionization gas”, which optionally comprises any suitable mix of atomic and/or molecular species (including a single species) which can be ionized to generate the cold plasma. Typical ionization gas mixtures include one or more noble gasses optionally mixed with other species, for example, molecules of nitrogen, oxygen, and/or water.

The tissue target to which cold plasma is delivered, in some embodiments, is internal to a living body. Optionally, the target is outside a living body, and optionally the target is not a portion of a living human body. For example, the target is optionally a calibration target, e.g., a target instrumented to characterize what plasma is produced at different settings of the plasma delivery device, optionally including different parameter settings of the plasma delivery tip; for example: different dielectric barrier thicknesses, different gas delivery lumen diameters, and/or different discharge electrode widths. Additionally or alternatively, the target is an assay target; e.g., a target of an in vitro and/or ex vivo assay of cold plasma effects (e.g., under conditions of different parameter settings of the plasma delivery tip) on one or more types of, for example: tumor cells, pathogen cells, infectious particles (viruses or prions), healthy cells, and/or tissue samples.

Cold plasma is generated in a non-equilibrium state, and its ionization state rapidly decays as charged species interact with each other, with other species in the ionization gas, and/or with the environment.

In some embodiments, a plume of cold plasma (of, e.g., about 1-20 mm in length) is generated from a high-voltage discharge electrode operated within an ionization gas environment created near the target. A potential advantage of generating plasma is such close proximity to its target is reduction of plasma degradation due, e.g., to interaction with conduit walls. Characteristic of its non-equilibrium state, cold plasma is low-ionized. Ionization is estimated, for some cold plasmas, as being (within a factor of about 10) about a part per million and/or 10¹¹-10¹³ electrons/cm³. The generated plasma is carried on toward the target by flowing of the ionization gas.

These operating conditions place potentially conflicting constraints on device design.

One constraint is size. Body-internal access allowing plasma application to the tissue target is optionally facilitated by the use of a relatively small-diameter (e.g., 5 mm or less, 6 mm or less, 7 mm or less, or another diameter) distal tip of a plasma generation device (at which tip the plasma is also generated). The small-diameter plasma-generating tip itself may be introduced to the target, for example, using a catheter sheath and/or endoscope working channel.

Another constraint is temperature. Plasma generation, in some embodiments of the present disclosure, comprises delivering electrical power into a high-voltage gradient electrical field, through a portion of which flows a gas comprising one or more atomic and/or molecular species susceptible to ionization. In brief, the steep voltage gradient of the electrical field tears atoms apart into ions and freed electrons. This can in turn generate a cascading effect as high-energy freed electrons transfer portions of their energy to other, still-bound, electrons, freeing them as well. Freed electrons move at a high temperature (potentially several thousand degrees Kelvin); however, they provide very little of the plasma's thermal mass. The plasma is considered “cold” when the much heavier atomic ions themselves remain at about room temperature (moving relatively slowly). Typical targets for cold plasma generation in medical applications are to maintain the bulk plasma temperature at 40° C. or less (e.g., below protein denaturation temperatures), or 50° C. or less. In some embodiments of the present disclosure, plasma temperature is less than body temperature, and optionally room-temperature, for example, in a range of 20-35° C., and optionally in a range of about 24-25° C. This thermal condition may be enhanced, for example, by removing heat from the system fast enough to overcome the heating effects of input electrical power. One way of doing this is to maintain continuous flow of the supply of the ionization gas (which thus becomes its own coolant). In some embodiments, there is also provision for removing waste ionization gas.

Another constraint is electrical safety. High voltages tend to produce high currents, resulting in a potentially serious safety issue for medical applications. In some embodiments, the safety issue is reduced by generating plasma through the method of dielectric barrier discharge (DBD). In this method, a dielectric barrier (e.g., an electrical insulator) is positioned between the high voltage electrode and ground. Electrical-barrier discharge may then be accomplished using a pulsed voltage, e.g., comprising pulses at radio frequency and/or microwave frequency. Ionizing gas flowing along the dielectric barrier, opposite the side of the discharge electrode, is thereby subjected to changing electrical fields capable of stripping some electrons from their atoms, generating free electrons. Free electrons gather energy from the electrical field to form a discharge current; a displacement current is also generated through the dielectric material. There is relatively little real power transmission into the ionization gas, so that the generated plasma, in bulk, remains “cold”, even though some of the electrons are accelerated to higher temperatures.

According to some embodiments, the pulses are configured not be dangerous to the patient because the inner electrode is insulated. For body-internal use, electrical ground is optionally provided through the tissue itself—since electrical barrier discharge currents are low. In addition, the high frequency pulses assume high voltage for very short duration (e.g. on a nanosecond time scale), making them considerably safer to patients as compared to long duration pulses. RF pulses having MHz frequency potentially require lower voltage amplitudes to initiate the plasma compared to separated single voltage pulses or RF pulses at 10 kHz frequency, therefore adding safety to the device.

It should be understood that the dielectric barrier is not per se essential to the generation of plasma. In some embodiments, a dielectric barrier layer is omitted, short-circuiting the ionization gas to the discharge electrode. In such embodiments, the safety provided by the current limiting effects associated with DBD is not present. Optionally, protection uses a control-based method; for example, detection of over-current events (arcing) coupled to rapid shutdown of supplied high voltage.

Embodiments described herein include the dielectric barrier layer, but it should be understood that, at least from an electrical standpoint, the dielectric barrier layer is optionally removed. This, of course, removes from the embodiments features which relate to changing characteristics (e.g., the thickness) of the dielectric barrier layer. Features described herein which rely on mechanical attachments of the dielectric barrier layer to induce change in lengths and/or thicknesses of other features are optionally preserved, e.g., by substituting a material (e.g., a metal) or design (e.g., a perforated polymer) which does not act as a dielectric barrier layer. Potential advantages of removing the dielectric barrier layer include reducing overall thickness (outer diameter) for a same gas delivery lumen size (inner diameter), and reduction of breakdown voltage, allowing use of a lower voltage at the discharge electrode.

An aspect of some embodiments of the present disclosure relates to plasma device tips with dynamically configurable functional parameters affecting the generation and/or delivery of plasma (that is, parameters of the plasma device tips are modifiable to adjust cold plasma production). Potential advantages of dynamically configurable functional parameters include: maintaining electrical and/or thermal safety, tuning to the requirements and/or constraints of a given target and/or target site, and/or tuning to the use of a particular ionization gas (e.g., a particular mixture and/or pressure of atomic and/or molecular species).

In some embodiments, a plasma device tip is configured to allow varying one or more plasma plume-generating parameters of the device; for example:
A gas delivery lumen diameter,
A dielectric barrier resistance and/or impedance,
Discharge electrode geometry,
Discharge electrode placement (relative to an ionization gas stream), and/or
Direction and/or speed of ionization gas flow

Adjustments are optionally over a range of a factor of about ±5% around a center value, ±12% around a center value, ±25% around a center value, ±50% around a center value, or another range. In some embodiments, the adjustments are performed using independent adjustment mechanisms. In some embodiments, a single adjustment mechanism coordinately adjusts two or more plasma-generating parameters. In some embodiments, plasma delivery tip size (e.g., diameter) is adjustable (e.g., expandable, optionally expandable from a minimum size used for delivery) once at the site of cold plasma delivery, changing its electrical characteristics to be more suitable for safe intrabody cold plasma delivery. In some embodiments, one or more plasma delivery tip characteristics are adjusted to tune plasma plume generation to the specific environment of treatment, and/or to a specific target treatment. A notional “perfect” setting for producing plasma within a certain environment may be variable and/or unknown in advance—e.g., affected by factors such as rates of thermal buildup and dissipation, target geometry, and/or target accessibility.

In some embodiments of the present disclosure, plasma delivery tips are configured to be navigable into intrabody target regions through lumens and/or apertures of, for example: about 15 mm or less, about 10 mm or less, about 5 mm or less, about 4 mm or less, or about 3 mm or less. A diameter of a gas delivery lumen delivering ionization gas to be ionized to plasma at the plasma delivery tip, and/or delivering the ionized gas itself as a plasma plume leaving the plasma delivery tip optionally ranges between about 0.4 mm and 8 mm. The portion of the plasma delivery tip which generates and shapes the plasma plume is optionally between about 4 mm-30 mm in length. Longer lengths are optionally used with correspondingly higher discharge voltages, to prevent dielectric breakdown.

It may be appreciated that limiting the physical size of a plasma-generating tip of a plasma delivery device (e.g., to a size insertable through or as a catheter, and/or over the working channel of an endoscope) in turn places constraints on the plasma generating parameters of the device, and/or on the thermal, ionization, and/or geometrical characteristics of the plasma plume generated.

As tip size decreases, temperature tends to increase for a given amount of delivered ionizing electrical power (that is, as power density increases); for example, insofar as the power is delivered to a more concentrated area. A smaller tip size, moreover, limits gas flow through the tip, which also potentially contributes to an increase in temperature due to loss of coolant effects. Factors such as these tend to push operating temperatures towards the top of the allowable range (e.g., towards 40° C.). However, as tip size (and thus, its thermal mass) decreases, thermal properties of a potentially variable operating environment become increasingly important to determining the equilibrium temperature of the device during operation. For a device having static electrical parameters, it may be difficult to ensure delivery of therapeutically effective amounts of plasma while maintaining an adequate margin of thermal safety for all such operating environments.

It is a potential advantage to maintain power delivery near to current thermal limits (while remaining below them). As thermal limits change (e.g., as a function of thermal characteristics of the environment and/or device), a target power delivery level may correspondingly change. One way of affecting power is to adjust an axial length of the portion of a generated electrical field which is effective for generating plasma. This is at least partially accomplished, in some embodiments of the present disclosure, by increasing/decreasing a discharge electrode length.

Another way of affecting power is to increase a voltage delivered to the discharge electrode. However, in embodiments operating by dielectric barrier discharge, the voltage should be set below a breakdown voltage of the dielectric barrier.

As probe size decreases, requirements on dielectric barrier thickness potentially limit minimum practical device size (e.g., minimum diameter). Exceeding the breakdown voltage potentially leads, e.g., to temporary device shutdown and/or creates a safety issue. Making the dielectric barrier too thick may interfere with plasma generation at lower voltages, and/or lead to reduced gas lumen size (i.e., in embodiments where physical thickness of a dielectric barrier is increased to increase its breakdown voltage). Materials suitable for use as the dielectric barrier include materials with a dielectric constant of up to about 6-8, and/or a dielectric strength of about 10-10 kV/mm or higher. In some embodiments, dielectric barrier wall thickness is in a range of between about 0.07 mm and 1.5 mm.

Rigid dielectric materials include ceramics, quartz, and certain glasses (e.g., Pyrex™) Potentially elastic dielectric materials include, for example, PEEK (polyether ether ketone), PTFE (polytetrafluoroethylene), ABS (acetonitrile betaine styrene), TPU/TPE (thermoplastic polyurethane, or thermoplastic elastomers more generally), nylon, and/or PVC (polyvinyl chloride).

In some embodiments of the present disclosure, dielectric barrier thickness is adjustable; e.g., to allow matching barrier characteristics to those needed to work with a currently selected discharge voltage. The barrier thickness can be adjusted by adjusting layer thickness (e.g., of an elastic barrier material), and/or by adjusting layer numbers (e.g., of elastic and/or rigid barrier materials).

Energy discharge from the discharge electrode can be affected by how long or wide the ionizing zone is. For example, a longitudinally shorter discharge electrode potentially produces less ionizing effect than a longer one. Optionally, this is adjusted in some embodiments by adjusting a length of the discharge electrode (e.g., how much of the distal end of a core conductor of a coaxial cable, or another discharge electrode design, is unshielded).

Additionally or alternatively, in some embodiments, the positioning (distance and/or angulation) of the discharge electrode relative to a stream of gas is adjusted. This optionally includes positioning the discharge electrode outside of the conduit through which ionizing gas is being introduced, e.g., by adding a control member that allows the discharge to be advanced at least a few millimeters or centimeters beyond an aperture through which ionization gas is being supplied. This may be the same aperture as the one used to deliver the discharge electrode itself, or a different one. As it is advanced beyond the confines of a lumen of a plasma delivery tip or working channel, a discharge electrode may change its shape and/or orientation. For example, it may comprise a superelastic alloy which is configured to assume a preset shape upon release from confinement.

The ionizing zone then becomes, in part, a function of where the flow of ionization gas intersects with portions of the discharge electrode. If the ionization gas flows approximately perpendicular to a longitudinal axis of the discharge electrode, there may be a relatively short ionizing zone. If the ionization gas flows approximately along the longitudinal axis of the discharge electrode, there may be a relatively long ionizing zone. Apart from its length (that is, length along a longitudinal axis), a discharge electrode may be relatively wide or narrow. Depending on the flow of ionization gas, a relatively greater discharge electrode width may establish a correspondingly larger ionization zone.

Additionally or alternatively, control of the zone of intersection may be accomplished by redirecting the flow of ionization gas, by positioning the discharge electrode closer to or further from the flow of ionization gas, and/or by orienting the discharge electrode so that the flow of ionization gas intersects a larger or smaller extent of the discharge electrode.

It is noted that directionality of the flow of ionization gas outside a lumen which delivers it is optionally controlled on either of its “ends”.

On the proximal side (the aperture from which flow issues), a main direction flow can be redirected by re-orienting an aperture and/or baffle.

On the distal side (out in the environment), flow is affected by nearby structure; for example, by the proximity and/or relative orientation of a tissue surface comprising a treatment target. As it approaches a surface, flow may be converted to a direction which flows more nearly parallel to the surface—even if it begins at a substantially perpendicular angle to it. A surface of the environment may also act in part to confine the flow of plasma. For example, plasma may be generated on either side of a plate-shaped electrode (flat, but relatively wide and long) over which ionization gas is flowing. If one side is brought close to a tissue target, the surface of that target (and surrounding area) tends to confine plasma generated on that side, potentially increasing its concentration.

Cold plasma concentration can be affected by how long (as a time duration) the flow of ionization gas is within the ionizing zone, making it a potential function of not only the geometry (e.g., length and width) of the ionizing zone, but also the velocity of the gas flow. For a given pressure of supplied ionization gas, a lower diameter tip lumen potentially lowers the concentration of plasma ions as the flow of gas speeds up. However, the final plasma concentration may tend to increase by limiting the diameter of the lumen. Conversely, an increase in the flow of ionization gas (diameter remaining constant), potentially increases power while reducing plume temperature. Accordingly, an optimal diameter adjustment for plasma concentration is potentially found at neither the smallest nor the largest diameter, as may be determined by trial adjustments and observations.

When plasma is generated within a lumen—for a given lumen diameter (other plasma-generating parameters such as ionization gas composition and/or flow rate being equal), there is a corresponding distance—typically equal to several lumen diameters—along which current and power carried through the plasma plume (if otherwise unquenched) are approximately constant. Past this length, power delivery drops. Similarly, plasma temperature tends to be approximately constant along an initial portion the plasma plume. However, near the tip, tapering in the plasma plume may tend to increase current density and/or temperature. The plasma regions of more constant current, power, and/or temperature are potentially more preferred for use in treatment—e.g., more controlled in their parameters, and potentially safer and/or more efficacious.

Plasma generated outside the lumen (e.g., at an intersection between unconfined flow and a suitably positioned discharge electrode) may mirror the same general observation in lumen-generated plasma: that plasma at certain distances closer to the discharge electrode is cooler or otherwise more suitable for use in tissue treatment than plasma a short distance away. Accordingly, a potential advantage of using an “extralumenal” electrode (that is, a discharge electrode positioned within a stream of ionization gas which is unconfined by its delivery lumen) is that it can be brought arbitrarily close to the tissue target.

Moreover, in some embodiments, adjustments of the geometry of the discharge electrode are made which help control distances from the electrode at which generated plasma contacts tissue targeted for treatment. For example, a protruding abnormal tissue target may be treated using a discharge electrode which is shaped with a concavity that can be positioned to partially encircle the protrusion. Conversely, a curved interior surface of a body organ (e.g., the inside of a colon, bladder, or other hollow organ) may be accommodated by providing a discharge electrode which is shaped to a convexity that can be positioned where it follows the interior curvature of the surface.

The geometry of plasma generating elements, e.g., their sizes, symmetries and/or relative placements, can also affect plasma plume shape. For example, increasing lumen diameter and/or ionization gas flow tends to result in higher power (resulting in more ionization) and a longer plasma plume. A greater electrode width (in a direction along a proximal-to-distal direction of ionization gas flow) also tends to result in higher power. A thinner or lower-resistance dielectric barrier results in lower breakdown voltage.

To monitor the effects of modifying plasma generating parameters on plasma generation itself, there are at least two general methods which may be applied.

In the first method, the power being delivered by an electrical power supply providing voltage to the discharge electrode can be monitored. A “targeted” power level can be selected, e.g., based on experiments which correlate treatment effects with power levels, and/or which correlate temperature levels with power levels. If power is found to deviate from the target level, adjustments can be made until the target power level is once again achieved. It should be noted that this does not require additional elements to be added to the plasma deliver tip itself.

Additionally or alternatively, direct temperature monitoring may be performed, e.g., by use of a thermistor, thermocouple, and/or spectroscopic probe provided at a suitable location. The probe may be placed, for example, within a lumen of a plasma delivery tip, and/or upon a discharge electrode of a plasma delivery tip. Optionally, the temperature monitoring probe is brought to near the site of plasma treatment as a separate tool inserted via the lumen used to deliver ionization gas and/or the discharge electrode. Optionally, the temperature monitoring probe is brought to near the site of plasma treatment via an auxiliary channel, for example, a working channel of an endoscope other than the lumen used to deliver ionization gas and/or the discharge electrode. Spectroscopic monitoring may also be used to measure concentrations of reactive species in the plasma (according to their particular emission spectra). Optionally this information is used to guide the tuning of plasma generating parameters.

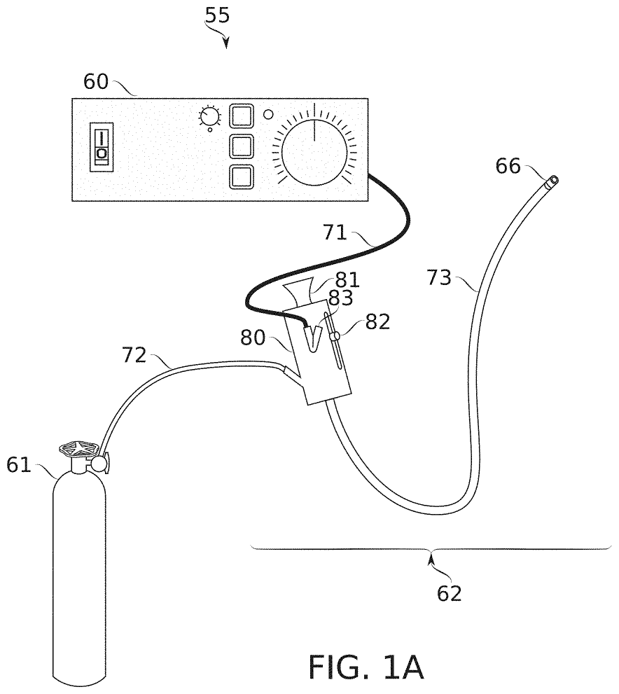

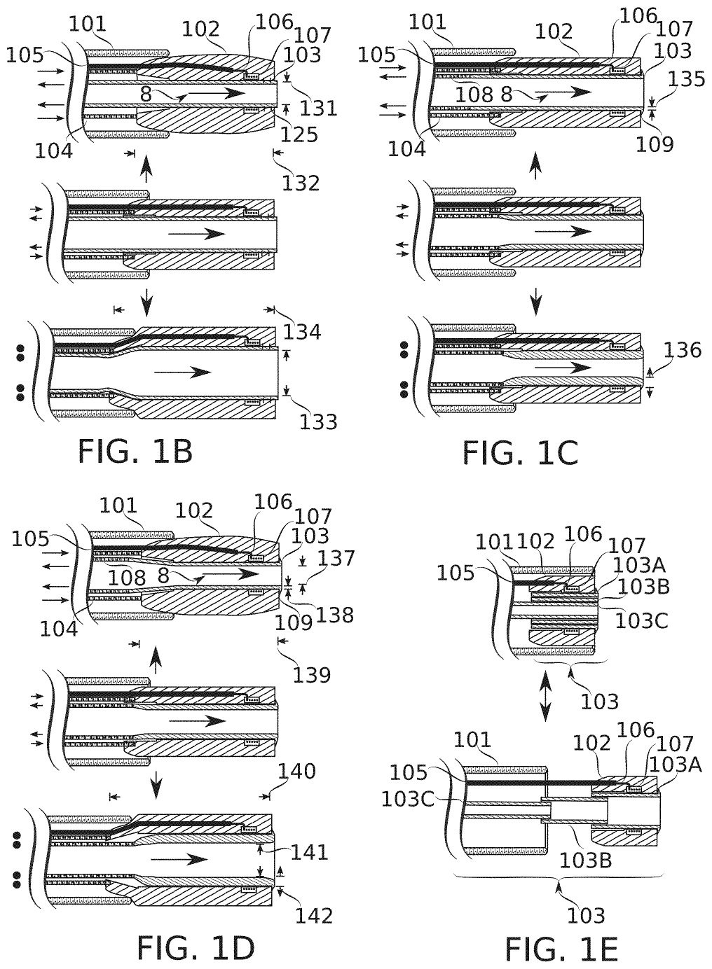

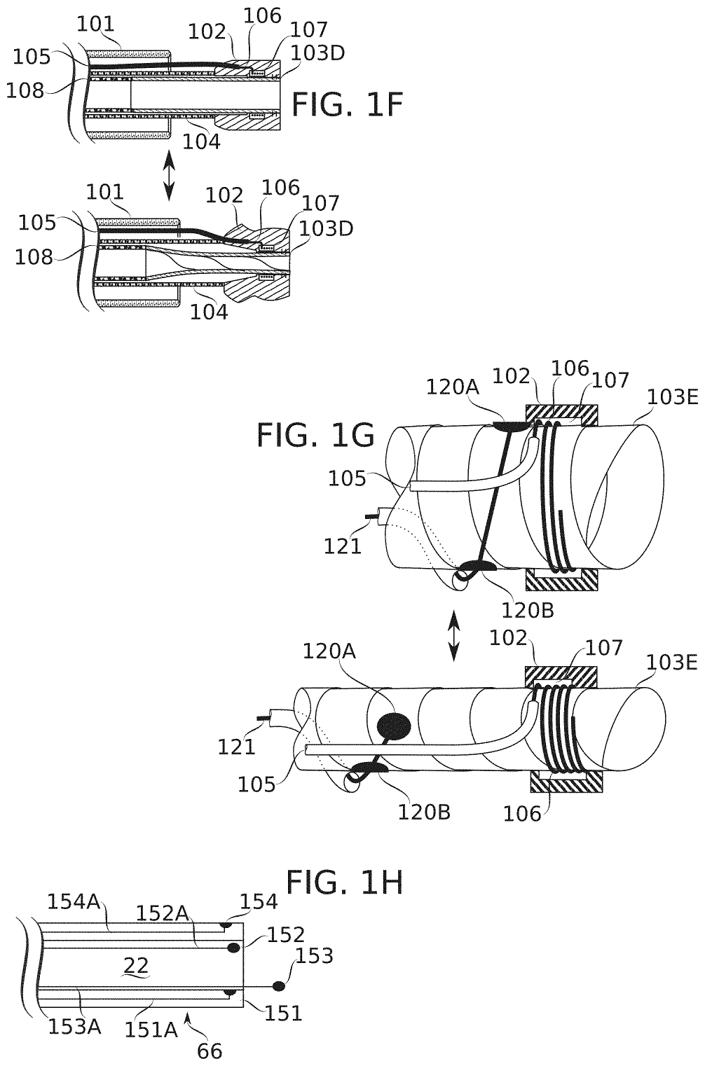

For simplicity, most examples described herein omit specific indication of the location of sensing devices used in monitoring. However, it should be understood that any of them may be equipped with a thermal and/or spectral sensor, for example as generally described in relation to FIG. 1H, herein.

Changes made in response to monitored deviations in power and/or temperature from targeted levels vary depending on the particular embodiment. Principles of making these changes are further discussed in relation to the particular embodiments. It should be understood that these principles, even when described in relation to a particular embodiment, apply also to other embodiments which share a relevant feature. The principles may relate, for example, to effects on plasma generation for adjustments of diameters, lengths, thicknesses, relative distances, relative orientations, ionization gas flow rates, and/or applied voltages.

An aspect of some embodiments of the present disclosure relates to the configuration of plasma delivery tip elements for use in navigation and/or penetration of the plasma delivery tip through tissue.

In some embodiments, an electrical power conduit (e.g., a coaxial cable) which interconnects a discharge electrode with a high voltage power supply also acts as a steering control, e.g., steering an orientation of the plasma delivery tip according to tension exerted on the electrical power conduit.

In some embodiments, an electrical power conduit (e.g., a coaxial cable) which interconnects a discharge electrode with a high voltage power supply also acts as a guidewire. For example, the electrical power conduit can be advanced from the plasma delivery tip to select among different courses of potential advance of a plasma probe of which the plasma delivery tip is a part.

In some embodiments, a discharge electrode is shaped to assist in the advance of a plasma probe of which the plasma delivery tip is a part. For example, the discharge electrode is capped with an atraumatic tip (e.g., comprising the dielectric barrier layer) shaped to help guide advance of the electrical power conduit when used as a guidewire. Alternatively, the discharge electrode is capped with a sharpened tip (e.g., comprising the dielectric barrier layer) suitable for penetrating tissue and/or blockages.

In some embodiments, a lumen through which ionization gas is delivered is beveled to a sharpened tip, suitable for penetrating tissue and/or blockages.

An aspect of some embodiments of the present disclosure relates to methods of constructing a stiff, but small-diameter discharge electrode assembly. In some embodiments, a discharge electrode assembly is constructed based on a coaxial cable by stripping away outer insulation and flexible shielding from a distal portion of the coaxial cable, then replacing the flexible shielding with stiffer shielding (e.g., a metal tube), while leaving a distal portion of a central conductor of the coaxial cable unshielded. The unshielded portion of the central conductor is provided with the dielectric barrier material, optionally shaped to a point to help use of the discharge electrode assembly for navigation and/or penetration through tissue. Optionally, outer insulation is provided to electrically insulated the stiffer shielding.

An aspect of some embodiments of the present disclosure relates to gas return channels of a plasma delivery tip.

When a plasma plume introduces gas to an intrabody space, there is potentially a buildup of gas volume and/or pressure. In some embodiments, a plasma delivery tip is provided with return channels configured to relieve this buildup (passive return of gas). In some embodiments, a proximal side of the gas return channel is provided with a connector allowing a source of negative pressure (suction) to be attached to assist and/or induce the return of ionization gas.

In some embodiments, the return channels are helically shaped. This provides a potential advantage for cooling, insofar as gas which has been warmed by plasma generation cools somewhat upon interaction with the environment. The returning gas can also absorb some heat from the plasma delivery tip. Return along a spiral path increases the surface area along which such heat exchange occurs, potentially increasing the efficacy of returning gas as a coolant.

An aspect of some embodiments of the present disclosure relates to sleeves for plasma delivery tips that protect the plasma delivery tip as it travels through a working channel.

In some embodiments, a plasma delivery tip is sized for delivery through a working channel of a device which might optionally be used for other tools as well during the procedure. Accordingly, the use of the plasma delivery tip comprises advancing the plasma delivery tip distally through the working channel.

With plasma delivery tips having relatively complex (that is, non-circular and/or comprised of a plurality of free endings), geometrical arrangements of distal features, it is a potential advantage to protect the plasma delivery tip with a sleeve. Using a sleeve, however, introduces a potential disadvantage, by occupying working channel space. This causes the plasma delivery tip itself to need a narrower design, potentially increasing resistance to the flow of ionization gas therethrough, and/or reducing the cross-sectional area that a plasma plume delivered by the plasma delivery tip.

Nevertheless, a sleeve may have important functional features related to the electrical functioning of a plasma delivery tip. In some embodiments, a provided sleeve comprises a dielectric material. Adding the extra thickness of dielectric material potentially prevents voltage in the electrical conduit which transmits high voltage to the discharge electrode from itself inducing plasma discharge. The sleeve may also help to exclude gas from seeping back into the working channel around the ionization gas supply tube, where it may contribute to the ectopic generation of plasma. It should be understood that any of the embodiments described herein is optionally provided with a dielectric insulating sleeve which may extend partially or completely along a length of high-voltage conductor used to bring voltage to a plasma delivery tip where it is used to generate plasma. The sleeve may moreover be sized to have an outer diameter that fills a lumen used to transport the plasma delivery tip to its operating location within a body lumen, so as to provide a seal against the retrograde transport of plasma—further helping to prevent ectopic plasma discharge from occurring. In some embodiments, there is no such seal, and indeed, there may be intentional counterflow of ionization gas back proximally along the sleeve. In those embodiments, the sleeve is optionally designed to provide a dielectric thickness that prevents voltage carried within the sleeve from inducing ectopic plasma generation. Optionally, the sleeve is particularly provided and/or thickened in regions which have reduced electrical isolation for another reason. These regions may comprise, for example, regions where ground shielding of coaxial cabling is weakened and/or not provided; e.g., to reduce use of space, to allow making electrical connections, or for another purpose.

An aspect of some embodiments of the present disclosure relates to plasma delivery tips comprising a plurality of plasma generation sites which operate together to provide an increased area of plasma treatment.

More particularly, this aspect relates to embodiments provided with a plurality of plasma generation sites, with the plasma plumes they generating being preferably directed to locations that complement each other to complete an area of coverage.

These features provide a solution addressing the problem of matching plume size to target size.

The problem arises in part as a consequence of miniaturization which reduces a plasma delivery tip's size to a 2-20 mm diameter (typically) suitable for intralumenal use within a body cavity. For such intralumenal (e.g., endoscopically performed) procedures, it may be readily understood that an end target (e.g., a target comprising abnormal tissue such as tumorous, and/or infected tissue) is potentially considerably larger than the size of the access-way used to reach it with a treatment tool such as a plasma delivery tip.

The problem also arises in connection with the practical matter that for any particular geometrical configuration of a plasma delivery tip, plasma may actually only be validated for use within a relatively narrow range of plasma generating parameters. In particular, that range may include a relatively narrow range of plasma plume sizes (e.g., diameters and/or cross-sectional shapes).

Plasma generation may not occur dependably (or at all) outside of this range; or it may occur but with unknown or insufficient generation of potentially therapeutic plasma species. In general, simply scaling up a small plasma generating tip to a larger plasma generating tip introduces such a large change in plasma generating characteristics that at some point it effectively must be re-validated as a new design. Scaling of this type may not even be practical—for example, because of access-way size constraints, and/or because increased size can increase requirements on breakdown voltage levels (discharge electrode voltages) beyond what is reasonable and/or feasible.

In some embodiments of the present disclosure, a plurality of plasma generation sites are provided, each placed where it generates plasma within an area of gas flow that other plasma generation sites do not generate plasma within, or at least do not generate plasma in sufficient concentration within.

Different plasma generation sites are at least distinguished by comprising separate discharge electrodes. The electrodes may be electrically isolated from one another (e.g., each driven by a separate power supply), or (more simply), electrically interconnected by an electrical conduit which does not itself act as a plasma-generating discharge electrode.

In some embodiments, different plasma generation sites are also distinguished by separation of ionization gas into different flows. For example, there may be provided a plurality of outlet apertures from a gas delivery tube, with each outlet aperture being provided with its own discharge electrode. Additionally or alternatively, there may be provided a plurality of ionization gas delivery tubes, either separately supplied with ionization gas (e.g., from a pressure sources separately regulated at a proximal end of the gas supply tube), or joined in common to a main ionization gas delivery tube to form a manifold.

The plurality may also be compound; for example, an outlet aperture of a plurality of outlet apertures may itself be provided with a plurality of discharge electrodes, and/or a gas supply tube of a plurality of gas supply tubes may itself be provided with a plurality of outlet apertures.

In some embodiments, each plasma generation site generates plasma according to substantially the same parameters, e.g., the same outlet aperture size, the same rate of gas flow therethrough, the same discharge electrode geometry, and/or the same discharge voltage.

Optionally, a plurality of plasma generation sites are also provided with the same basic support structure design: for example, each site is located at the distal end of a tube which extends longitudinally from a distal end of a delivery sleeve, or each site is located in a lateral aperture of such a tube.

In some embodiments, sites having substantially the same parameters affecting plasma generation as such (e.g., gas flow, outlet aperture size/shape, electrode design and/or discharge voltage) are embedded in with different supporting structures. For example, one or more outlet may be oriented to direct a plume along a longitudinal axis of a distal part of the plasma delivery tube, with one or more other outlets may direct the plume oblique to the longitudinal axis, and/or perpendicular to the longitudinal axis.

The plurality of plasma generating sites may be pre-arranged so that they produce a pattern of individual plasma plumes that complement each other to generate a combined plume which is shaped to assure coverage of a certain area. The combined plume shape may be itself large enough to cover the “total” target area, or it may be shaped in such a way as to make plasma plume scanning easier and/or more reliable. For example, the combined plume may cover a linear region which can be scanned (e.g., by bending of a plasma delivery tube) through motions perpendicular to the linear region to gain area coverage. The combined plume may be formed as a ring circumferentially surrounding an arrangement of outlets, allowing it to be advanced longitudinally through a substantially tubular lumen, such as a lumen of an intestine.

Individual plumes of the combined plume are optionally combined by the use of a scanning motion. For example, a plurality of laterally-projecting plasma plumes may be rotated about a longitudinal axis to produce a ring-shaped plasma coverage area. In some embodiments, bending motions of a plasma delivery tube cause plumes arranged in a grid of substantially parallel plumes to cross into each others' previous coverage areas, producing a merged area of coverage.

An aspect of some embodiments of the present disclosure relates to plasma delivery tips comprising a plurality of plasma generation sites which are delivered in a first configuration, then rearrange to a new configuration which allows them to operate together to provide an increased area of plasma treatment.

In some embodiments, plasma generation sites rearrange upon deployment to a shape suitable for use in giving target coverage. The rearrangement can help to overcome mismatch between access-way diameter and target size. In some embodiments, plasma generation sites are separately mounted on tubes which advance from confinement by a sleeve and/or working channel to assume a splayed-out shape, capable of delivering a combined plasma plume that potentially covers a wider area than would be covered by operating the same plasma generation sites in their closer-packed delivery configuration. In some embodiments, the splayed-out shape is accomplished by increasing the spacing between plasma generation sites. In some embodiments, the splayed-out shape is substantially linear; e.g., a linear shape rearranged from a close-packed configuration that exists while the sites are confined within a substantially circular lumen of a sleeve.

Spacing increases are optionally implemented, e.g., by mounting each plasma delivery site at the end of a tube which bends slightly when released from sleeve and/or channel confinement so that a more spread-out configuration is reached. Optionally, the tubes themselves are predisposed to bend to the spread-out configuration. Optionally, a flexible truss is used that expands when released from confinement to help position the tubes in their spread-out configuration.

An aspect of some embodiments of the present disclosure relates to the modification of plasma plume shape to suit target geometry.

From where they exit a delivery tube aperture, plasma plumes made from jetting gas tend may tend to adopt a pencil-like shape, or another shape such as an expanding funnel. This shape is a consequence of many factors, for example: parameters of ionization gas, parameters of ionization gas flow, electrical parameters that generate the initial ionized species in the plasma, geometrical parameters such as electrode and plasma outlet aperture shape, and gas flow and/or electrical interactions of the plasma plume with its environment. Due to its velocity, a jetting gas plume has some potential advantages for projecting plasma to sites beyond the site of its generation.

However, there is no particular requirement that plasma be generated within a jetting flow of ionization gas. In some embodiments, for example, a general atmosphere of substantially static ionization gas (e.g., as may be established within a hollow body organ such as a bladder or an intestine) is optionally generated. Then, the plasma plume shape is potentially dominated by the shape of the discharge electrode—which is not necessarily itself positioned within the lumen used to deliver the gas. In such a situation, the discharge electrode itself can be positioned in direct proximity to a surface targeted for plasma treatment and operated. The discharge electrode may be curved to match a curvature of the surface, for example. The discharge electrode may be scanned over the surface, e.g., by rotation, and/or by bending motions of a tube used to deliver it.

In some embodiments, plasma generation is performed in a regime of ionization gas flow and discharge electrode positioning which is in between the two extreme states just described. For example, a jet of ionization gas may be broken up and/or redirected by the presence of surfaces in the environment, such as the target surface itself located distally to the out aperture from which the ionization gas issues. There may be a complete replacement of ambient gas with ionization gas, or partial.

Whether or not the replacement is complete, the plasma plume shape is still potentially affected by flowing of gas. In some embodiments, discharge electrodes are configured to be positioned in the redirected (e.g., laterally directed) flow of ionization gas, outside the tube that delivered the gas. The discharge electrode itself is optionally shaped to assist in the redirection of ionization gas, e.g., with baffles, and/or simply by acting as a barrier to flow in its own right. Optionally, a discharge electrode can be manipulated while the gas flow remains substantially in place. In some embodiments, a discharge electrode is configured to extend laterally from a longitudinal axis of a same tube as is used to deliver ionization gas. At a sufficient proximity to a target surface, the stream ionization gas is diverted laterally. Rotating the discharge electrode sweeps it through different circumferential portions of the lateral stream of ionization gas. Since plasma is generated where the ionization gas intersects the electrode, the sweeping motion of the electrode also generates an increase area of plasma coverage. Again, a shaped electrode is optionally used to suite a particular target surface shape; e.g., the electrode may be curved so that a convex surface of the electrode is brought against a complementary concave target surface.

Furthermore, there is no particular requirement that plasm be generated from a circular flow of ionization gas. In some embodiments, plasma is generated within a flow of ionization gas shaped by a non-circular outlet aperture, for example, an oval or slit-shaped outlet aperture. In particular, outlet apertures with a long axis and a short axis (e.g., a long axis at least twice as long as the short axis) provide a potential advantage by spreading the plasma plume into a more linear shape. The more linear shape can then be scanned in a direction perpendicular to its long axis to produce a larger area scanned per sweep. Also, offsetting sequential sweeps of the linear shape in a direction parallel to its long axis is potentially more readily controlled—e.g., with less of a tendency to leave coverage gaps between adjacent sweeps.

An aspect of some embodiments of the present disclosure relates to plasma delivery tips which are configured to distribute plasma to surfaces by use of “scanning” motions. In some embodiments, the scanning motion comprises rotation of an outlet aperture of the tip around a circumferential path. In some embodiments, an orientation of the outlet aperture is selectable and/or dynamically controlled, providing additional options for generating coverage of a target surface with plasma.

In some embodiments, provision is made for “scanning” one or more plasma plumes to cover a larger area than the cross-section of the plumes themselves provide. This potentially also helps to ensure that a target surface receives sufficient plasma coverage.

The scanning may be performed, for example, using control of the bending and/or advance of a gas supply conduit, sleeve for the gas supply conduit, and/or working channel. Additionally or alternatively, in some embodiments, the scanning is performed by rotating a gas supply conduit, sleeve for the gas supply conduit, and/or working channel.

It should be noted that any of the bending, advance and/or rotation is optionally dynamic during scanning (e.g., changes in the bending, advance and/or rotation themselves accomplish the scanning), and/or used to set a configuration of the plasma delivery tip which controls how plasma plumes are directed when another motion degree of freedom is used.

Adding plasma plumes, controlling the direction of those plasma plumes, and/or scanning those plasma plumes by moving them over a target area are all ways to potentially overcome limitations this practical consideration imposes. The same selected plasma generating parameters can be duplicated at a plurality of outlet apertures, to each similarly produce a plasma plume, and then all these plasma plumes combined to deliver plasma to a target area.

An aspect of some embodiments of the present disclosure relates to plasma delivery tips that combine membership in two or more of the following feature-defined plasma delivery tip groups:
Tips that allow reshaping to adjust parameters of plasma generation—for example by changes in dielectric barrier thickness, discharge electrode shape and or position, and/or plasma outlet aperture size. The tip reshaping may be controlled by an actuating control member (directly or by removal of a constraining element), or the tip may reshape automatically, e.g., as a function of temperature.
Tips that produce a plasma plume that can be actively redirected by remote manipulation while the tip is within a body cavity. For example, the redirection may comprise rotation of elements relative to an introducing lumen used with the tip, and/or bending of elements that produce the plasma plume.
Tips that comprise a plurality of plasma generating sites; e.g., sites defined by a respective plurality of electrodes and/or ionization gas supply tubes.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Plasma Treatment Device

Reference is now made to FIG. 1A, which schematically represents a plasma treatment device 55, according to some embodiments of the present disclosure.

Plasma treatment device 55, in some embodiments, comprises a high voltage power controller 60 and an ionization gas supply 61 interconnected to a plasma probe assembly 62. High voltage power controller 60 supplies ionizing voltage to plasma probe assembly 62 via cable 71 (which may be, for example, a coaxial cable, or another electrical conduit with controlled impedance and shielding along its length). Ionizing gas supply 61 supplies an ionization gas to plasma probe assembly 62 via tubing 72. The supplied gas may comprise, for example, one or more noble gases such as neon, argon, or helium; and/or other gas(es) suitable for ionization into a plasma plume. Optionally, cable 71 and tubing 72 are integrated into a single cabling unit which connects with plasma probe assembly 62. Optionally, high voltage power controller 60 and ionization gas supply 61 are integrally housed.

Plasma probe assembly 62, optionally comprises a handle 80. Handle 80 is optionally provided with controls 81, 82 for controlling actuation of probe conduit 73 and/or plasma delivery tip 66, for controlling functions of power controller 60, and/or for controlling ionization gas delivery from gas supply 61. Optionally, plasma probe assembly 62 physically integrates power functions and gas delivery functions into probe conduit without use of a dedicated handle. In some embodiments, probe conduit 73 includes both a lumen for delivery of ionization gas, and high voltage (e.g., a continuation of cable 71 and tubing 72). In some embodiments, probe conduit comprises a plurality of lumens, e.g., a lumen attached to gas supply 61 which delivers of ionization gas, and a lumen which scavenges (removes) of ionization gas, optionally under suction. In some embodiments, any one or more of the lumens of probe conduit 73 is optionally used as a working channel, by insertion of a tool (e.g., a tool as described in relation to FIGS. 10A-10C). Handle 80, in some embodiments, comprises one or more ports 83 for introduction of such tools into a lumen of probe conduit 73.

In some embodiments of the present invention, probe conduit 73 and plasma delivery tip 66 are sized and otherwise configured (e.g., safety-configured) for the delivery of cold plasma to an intrabody location.

Embodiments described herein relate to a number of different configurations of plasma delivery tip 66. In some embodiments, a plasma delivery tip 66 comprises a lumen configured to deliver ionization gas; and a discharge electrode positioned within the flow of ionization gas. The discharge electrode is moreover configured to receive a high voltage, is insulated from direct contact with the flow of ionization gas by a dielectric barrier layer, and otherwise is insulated from the environment (e.g., on sides away from the flow of ionization gas, if any) as necessary by an insulating sheath layer. Herein, several different embodiments of each of these elements are described, many with additional features such as controllable dimensions, shapes, thermal properties, and/or electrical properties. In general, features and elements described in connection with different embodiments herein should be understood to be optionally provided together, insofar as they are mutually compatible.

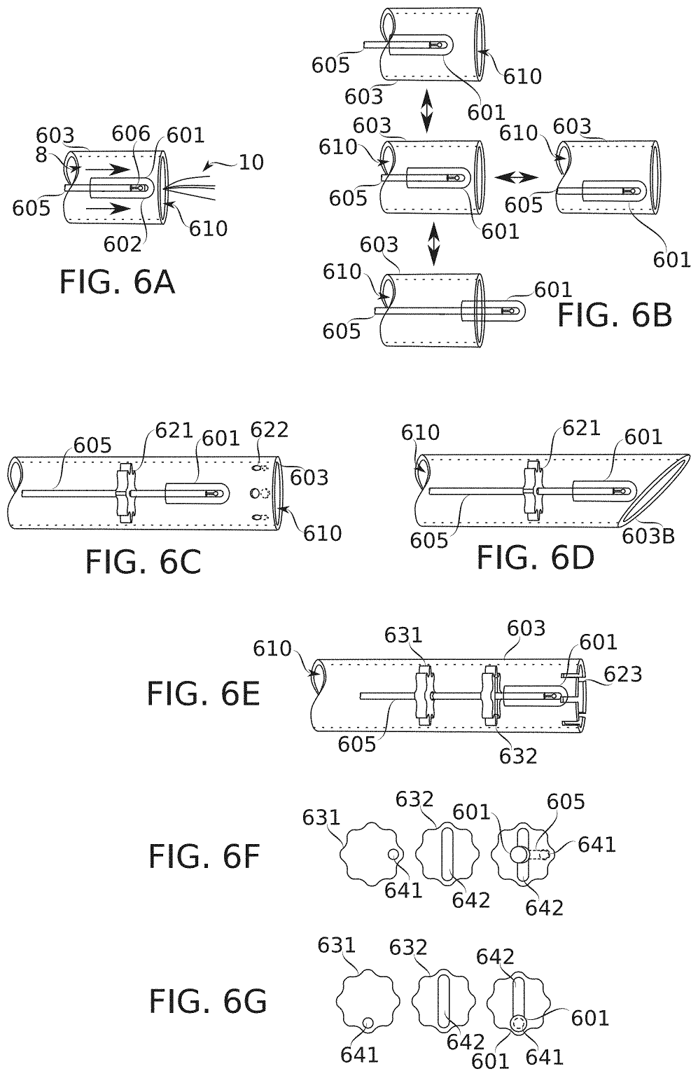

Two general classes of plasma delivery tip 66 include tips configured with a discharge electrode substantially surrounding the flow of ionization gas (described, e.g., in relation to FIGS. 1B-1F, 2A-5D) and tips configured with the flow of ionization gas substantially surrounding the discharge electrode (described, e.g., in relation to FIGS. 6A-11D. At least within each class, it should be understood that first elements described in one of the embodiments which do not intrinsically rely for their operation on the specific design of a second element are optionally combined with second element designs described in relation to another of the embodiments.

For example, there described herein different ways to make a discharge electrode change shape to accommodate a changing diameter of dielectric barrier layer. Discharge electrodes of a certain shape-changing type may be freely combined with shape-changing dielectric barrier layers of different types, so long as the discharge electrode itself is not itself part of the shape changing mechanism of the dielectric barrier layer. Even in cases where two elements interact (e.g., the discharge electrode constricts to compress the dielectric barrier), there may be disclosed herein more than one way of implementing the interaction substantially as described. It should be understood that elements may readily be combined across embodiments where mechanisms remain appreciably the same.

Another example of interchangeability among embodiments comprises the configuration of the insulating sheath layer. In some embodiments herein, insulating sheath layers are described as comprises a tube or ring, with other optional features such as being optionally actively or passively deformed in the radial and/or longitudinal direction, e.g., by operation of a control member or by movements of another element such as the discharge electrode and/or dielectric barrier layer; and/or having a hollow sized for receiving an electrode and allowing change to the shape of the electrode. In some embodiments, a plasma delivery tip is moreover configured with channels at least partially formed by the insulating sheath layer which provide for the removal of ionization gas and/or plasma after delivery (e.g., with a venting channel which also extends along probe conduit 73). It should be understood that such features of insulating sheath layer configurations are interchangeably separated and combined among embodiments—with each other, and with other different configurations of other elements such as configurations of electrodes and/or the dielectric barrier layer—insofar as such combinations are mutually compatible.

Additionally or alternatively, embodiments which are not individually stated to be provided with a dielectric sleeve are nevertheless optionally provided with one. Moreover, the sleeve, if it is individually mentioned, may be provided as a spacer- and/or tip-protecting type sleeve that acts as a fitting between a plasma delivery tip and a working channel through which the plasma delivery tip is advanced. The sleeve may furthermore be a sealing-type sleeve that prevents back-flow of ionization gas, or alternatively a sleeve with provision to act itself as part of a retrograde gas conduit, e.g., as described in relation to FIGS. 5A-5D.

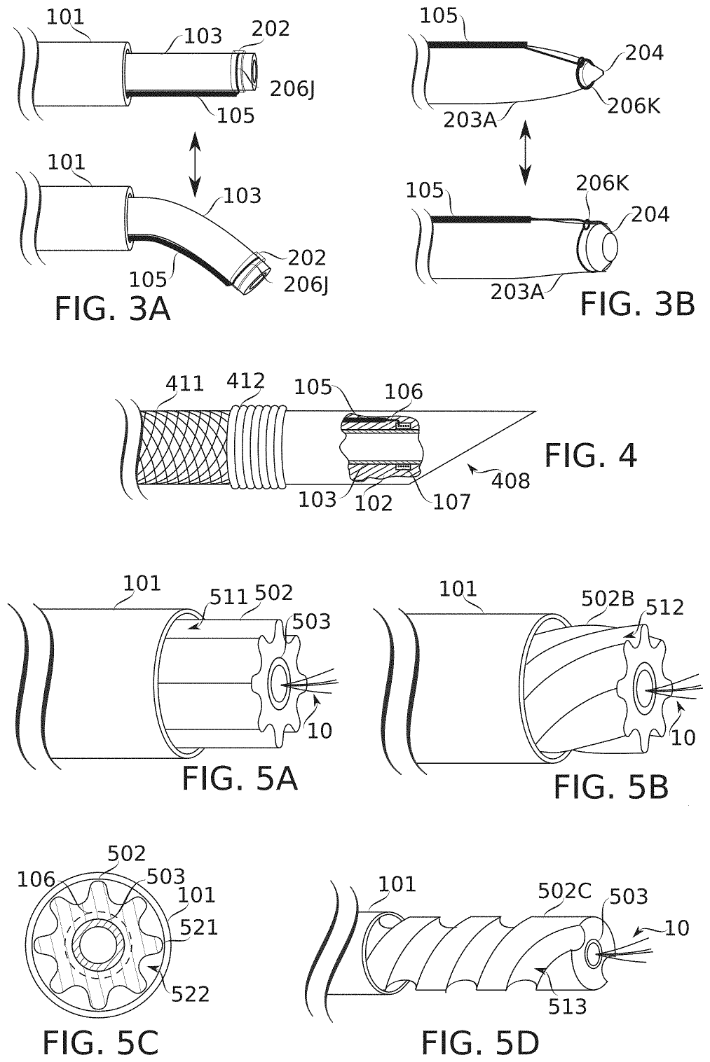

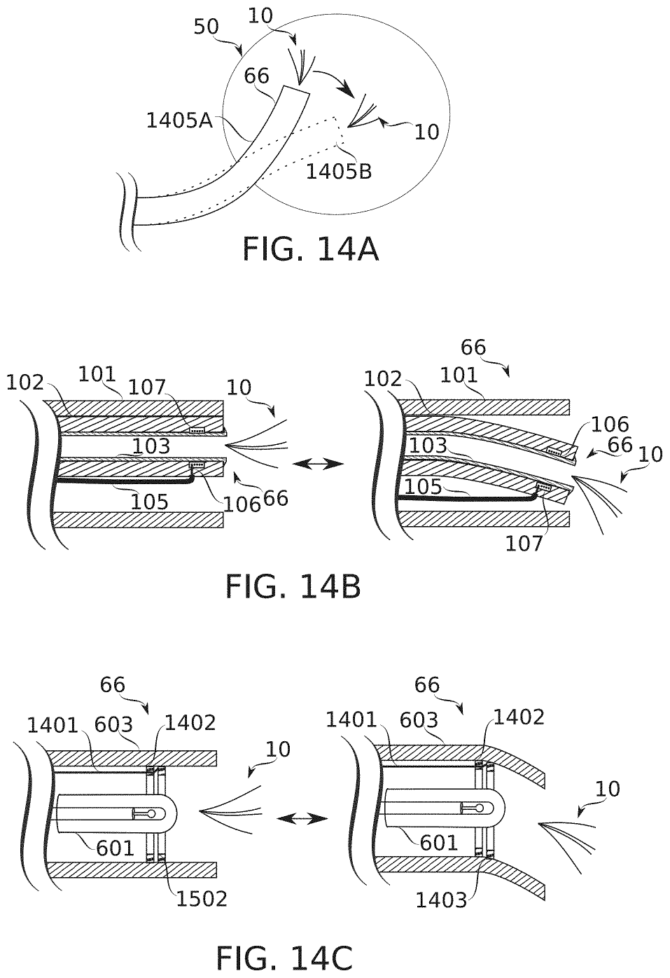

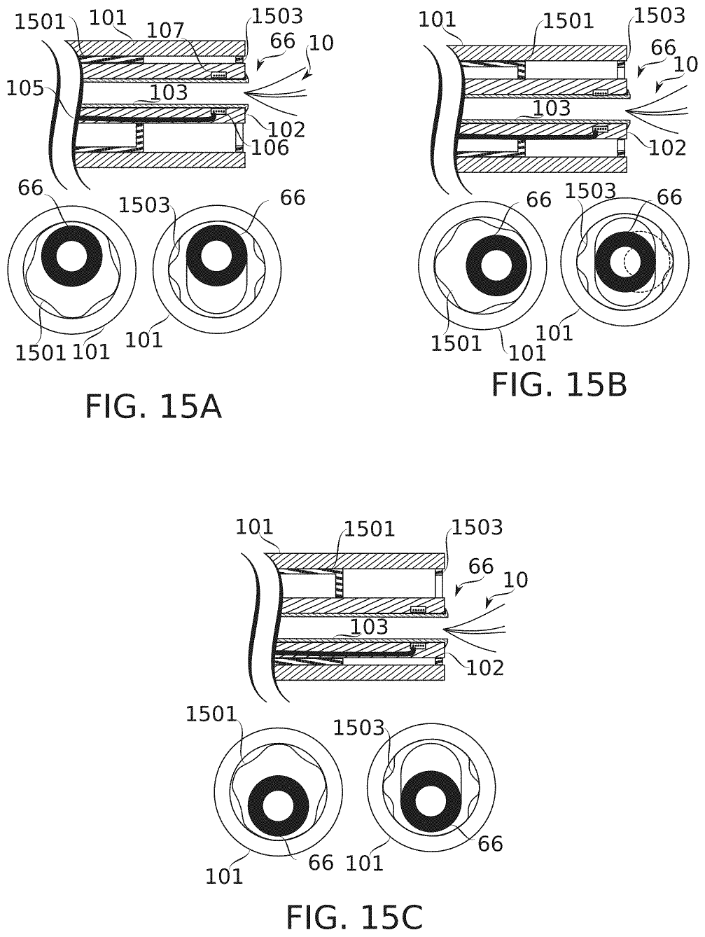

In some embodiments, one or more of these elements (or another element functional in plasma generation such as a power delivery conduit or portion thereof) is configured to perform one or more functions directed at device capabilities other than plasma generation. For example, an electrode, wire and/or cable is configured to induce steering movements of the device (e.g., FIGS. 3A, 14B-14C), to temporarily reconfigure the device for assisting device advance (e.g., FIG. 3B), and/or to act as a guide wire (e.g., FIG. 9). Additionally or alternatively, specific features of structural elements are described, including, for example, beveled tips (FIGS. 4, 6D), reinforcements (FIG. 4), and/or positioning supports (FIGS. 6C-6F, 15A-15C). It should be understood that such features are interchangeably separated and combined among embodiments—with each other, and with other different configurations of other elements such as configurations of electrodes, the dielectric barrier layer, and/or the insulating sheath layer—insofar as such combinations are mutually compatible.

FIG. 1A illustrates a plasma probe assembly 62 in a “stand alone” configuration, for example a configuration which may be itself used as a navigable catheter to reach an intrabody target. However, it should be understood that, in some embodiments, a plasma probe assembly 62 is optionally used together with another device; for example, by passing it through the working channel of an endoscope, or by inserting it through the lumen of a separate catheter. Plasma probe assembly 62 is illustrated as comprising a flexible probe conduit 73, however, it should be understood that probe conduit 73 is optionally stiff, and optionally straight or curved. Probe conduit 73 is optionally of any suitable length to reach its target.

Some embodiments of the present disclosure are described as comprising a sheath tube 101 with a lumen within which elements of a plasma delivery tip advance. Optionally, the sheath is a part of probe conduit 73. Optionally, the sheath is provided as the lumen of a device to which the plasma probe assembly is inserted, for example, a working channel of an endoscope or a separately provided catheter. Embodiments shown and/or described without a sheath are optionally provided and/or operated with one. Conversely, embodiments described with a sheath are optionally provided and/or operated “sheathless”, albeit that features specifically reliant on the sheath (e.g., using a portion of its lumenal space as a gas and/or plasma return pathway) may then be unavailable.

These remarks with respect to combinable elements and/or element features should be understood as also teaching principles which the examples in their aggregate illustrate; principles by which a person of ordinary skill in the art may recognize, using the teachings herein, combinations of elements and/or element features encompassed within the scope of these descriptions. These remarks and principles should not be construed as teaching, by any alleged omission, mutual exclusiveness of elements and/or element features.

### Adjustable Geometry Plasma Delivery Tips

Reference is now made to FIG. 1B, which schematically represents a plasma delivery tip 66 configured with an adjustable lumen diameter, according to some embodiments of the present disclosure.

Examples of a plasma delivery tip 66, in some embodiments of the present invention, comprise a generally tubular assembly about 0.5-5 cm long (e.g., about 1.5 cm long) in a proximal-distal direction, and about 1.5-6 mm in diameter (e.g., about 3 mm in diameter). The tubular assembly optionally comprises a plurality of layers—an inner dielectric barrier layer 103, and an outer insulating sheath layer 102.

Circumferentially interior to dielectric barrier layer 103 is a lumen through which a flow 8 of supply gas flows when the plasma delivery tip 66 is in operation. A discharge electrode 106 generally surrounds a circumferential portion of dielectric barrier layer 103. Discharge electrode 106 is supplied with electrical power at a high, alternating voltage (e.g., 500 V-2000 V) in order to ionize the gas of flow 8 into a plasma. The frequency of voltage alternation is selected, for example, from radio frequency (RF) to microwave frequencies. In some embodiments, the supply is provided over electrical conduit 105. Optionally, electrical conduit 105 comprises a coaxial cable, an outer conductor of which isolates the voltage supplied over the center conductor until reaching unshielded discharge electrode 106. Herein, it should be understood that embodiments described as comprising a coaxial cable optionally are embodied by replacing and/or augmenting the coaxial cable with another electrical conduit configured to deliver a voltage to a discharge electrode along an extent which is shielded and/or insulated to prevent unintended power losses and/or electrical discharge along its length. A coaxial cable provides a potential advantage for thin plasma delivery probes, insofar as it provides reliable electrical isolation of a high voltage conductor from the environment using optionally quite thin surrounding layers of material, e.g., a coaxial cable capable of maintaining at least a 1000 V RAMS isolation from the environment may be provided with an outer diameter of 1.1 mm.

Insulating sheath layer 102 is configured to keep discharge electrode 106 otherwise electrically isolated, sufficiently (even, for example, in a fluid environment) that at least the great majority (e.g., 90% or more) of real power transmitted from electrode 106 is directed into flow 8.

Optionally, electrode 106 is encapsulated within a space 107 defined by insulating sheath layer 102. Electrode 106 can be tightly encapsulated (e.g., with the material of sheath layer 102 cast around it). Optionally electrode 106 is loosely encapsulated, so as to allow movements of electrode 106 to accommodate changers in the geometry of layers 102, 103.

In some embodiments, a plasma delivery tip 66 is delivered by passage within the lumen of a sheath tube 101, for example, a catheter sheath and/or an endoscope working channel.

Optionally, insulating sheath layer 102 and dielectric barrier layer 103 are made of any suitable non-conductive material; for example, ceramic, Pyrex™, quartz, and/or a biocompatible plastic and/or rubber material. Examples include polyether ether ketone (PEEK), silicone rubber, and polytetrafluoroethylene (PTFE). In some embodiments, the two layers are connected but separate, e.g., optionally comprising different materials with different electrical and/or mechanical properties. The two layers 102, 103 are optionally manufactured as a single unit, and optionally of a single material, though this may introduce limitations on parameter selections such as electrical insulating properties and/or freedom of relative movement.

Considering the fairly small size of the plasma delivery tip 66 in some embodiments (e.g., about 3 mm diameter and 5 mm in axial length), it may be understood how the structure comprising insulating sheath layer 102 and dielectric barrier layer 103 may be constructed with a thinness and flexibility that allows it to be deformed quite substantially by the exertion of relatively small forces.

FIGS. 1B-1D illustrate embodiments which use changing relative longitudinal forces exerted from a proximal side of the plasma delivery tip 66 to control a lumenal width of dielectric barrier layer 103, a wall thickness of dielectric barrier layer 103, or both. Depending on the configuration, either or both of longitudinal compression and stretching can reduce lumenal width. Longitudinal compression may reduce lumenal width according to the Poisson effect, in which a material tends to expand in directions perpendicular to the direction of compression. Inward-directed expansion can cause the lumenal diameter to shrink. Longitudinal stretching may reduce lumenal width according to a tendency of a material to contract in directions transverse to the direction of stretching. Examples of devices using other operating principles to change one or both of these features are described, for example, in relation to FIGS. 1E-1G. Changes of dielectric barrier layer thickness may be, e.g., in a ratio (thin divided by thick) of as low as about 0.9, 0.75, 0.5, 0.25, or another ratio. Changes of gas lumen diameter (for example, at a most constricted point, or at a widest point) are optionally in a ratio (narrow divided by wide) of as low as about 0.9, 0.75, 0.5, 0.25, or another ratio.

In each of the panels of FIG. 1B, dielectric barrier layer 103 and sheath layer are optionally attached to each other at their distal ends (e.g., at attachment location 125; and attached, for example, by adhesive, heat welding, and/or pins), and/or mechanically constrained (e.g., by flanges 109 as shown in FIG. 1C) to prevent slipping completely past one another. However, along their bodies, the two layers 102, 103 are free to slide relative to one another.

In the middle panel of FIG. 1B, a moderate differential tension is applied by pulling proximally on layer 103, and pushing distally on layer 102. Optionally this is the “default” relative tension, which is set to keep the outer diameter of the plasma delivery tip 66 at a size (optionally, its smallest diameter) small enough to advance along a lumen of sheath tube 101.

Layer 102 is elastically deformable. Distally directed force on it is optionally transmitted from a relatively inelastic control member 104, comprising, e.g., a tube (e.g., as shown), cable, and/or other element that extends longitudinally through sheath tube 101 to interconnect between a control operated by a user, and layer 102.

Layer 103 is optionally elastic, but long enough that its longitudinal elastic deformations are widely distributed, and negligible over the length where layer 103 is encapsulated by layer 102. Optionally layer 103 is also attached to its own control member (for example, a control member 108 as shown in FIG. 1C).

In the top panel of FIG. 1B, a relatively high differential tension is applied by pulling proximally on layer 103, and pushing distally on layer 102. As layer 102 is compressed longitudinally (e.g., to a size indicated by arrows 132), part of its volume is displaced inwardly. Layer 103 is compliant enough to deform inwardly in turn, reducing the diameter of the lumen of layer 103 (e.g., to a size indicated by arrows 131). There may be a slight change in the wall thickness of layer 103 as a result, but the primary effect in the configuration shown is on lumen diameter. A narrow lumen is optionally selected in order to adjust a plasma jet, e.g., to increase its exit velocity and potentially its length. While increased resistance of a narrowed aperture may potentially reduce a net flow volume through the device (reducing some cooling effects), the increased velocity of flow may still potentially reduce transfer of heat into the device itself.

In the bottom panel of FIG. 1B, differential tension has been relaxed completely. This allows each of layers 102 and 103 to expand to its natural length (e.g., as indicated by arrows 134) and diameter, at least in portions unconstrained by sheath tube 101. Accordingly, the lumen of layer 103 also widens (e.g., as indicated by arrows 133). A wide lumen is optionally selected to create a larger internal working volume, potentially increasing the amount of ionizing power that the device can receive without resulting in an impermissible level of local heating.

In some embodiments, the plasma delivery tip 66 is designed primarily for configuration in a first, delivery mode which is delivered small enough to fit inside a selected size of a lumen of a sheath tube 101, and then a second, operating mode which expands to provide plasma with targeted temperature, electrical, and/or plasma generating characteristics. In some embodiments, a plasma delivery tip 66 is designed to allow variation of temperature, electrical, and/or plasma generating characteristics during plasma generation.

Reference is now made to FIG. 1C, which schematically represents a plasma delivery tip 66 configured with a tension-adjustable lumenal wall thickness, according to some embodiments of the present disclosure.

The device of FIG. 1C is configured in particular to produce changes in the wall width of layer 103. Layer 102 is relatively inelastic, and layer 103 is relatively elastic (and optionally shorter than as is shown in FIG. 1B), so that as layer 103 receives increased proximal force (upper panel), it is drawn thinner (as indicated by arrows 109). Optionally, inward collapse of the lumen of layer 103 is prevented by configuring layer 103 with a relaxed and unconstrained diameter which is somewhat larger than the inner diameter of layer 102.

With decreasing relative tension, (middle and lower panels), layer 103, correspondingly, gradually thickens (e.g., as indicated by arrows 136). This thickening tends to increase the resistance, impedance, and/or dielectric strength of layer 103, correspondingly increasing the breakdown voltage, and/or reducing ionizing power of voltage delivered to discharge electrode 106.

Reference is now made to FIG. 1D, which schematically represents a plasma delivery tip 66 configured with co-adjustable lumenal wall thickness and lumen diameter, according to some embodiments of the present disclosure.

In this embodiment, otherwise similar to the embodiment of FIG. 1C, layer 102 is elastically deformable enough so as to also substantially deform (e.g., under the longitudinal forces which modify the wall thickness of layer 103). Arrows 139 and 137 (upper panel), represent longitudinal compression and lumen shrinkage of layer 102 relative to the middle panel, as longitudinal stretching forces are increased. Arrows 138 (upper panel), represent wall thinning of layer 103 relative to the middle panel.

Arrows 140 and 141 (lower panel), represent longitudinal lengthening and lumen widening of layer 102 relative to the middle panel (as longitudinal are relaxed). Arrows 138 (lower panel), represent wall thickening of layer 103 relative to the middle panel as longitudinal forces are relaxed.

With respect to each of FIGS. 1B-1D, it should be understood that the layer configurations assumed with longitudinal forces relatively relaxed or increased are optionally offset and/or inverted. For example, the top panel in each case is optionally the “relaxed” longitudinal force panel, and the bottom panel optionally has the most longitudinal force exerted.

In some embodiments, radial forces replace and/or supplement longitudinally exerted forces to produce changes in layers 102, 103. For example, withdrawal into sheath tube 101 of a plasma delivery tip 66 as shown in the lower panel of FIGS. 1B and/or 1D optionally results in narrowing of lumenal diameter due to compression within sheath tube 101.

Reference is now made to FIG. 1E, which schematically represents a plasma delivery tip 66 configured with telescoping-adjustable lumenal wall thickness and lumen diameter, according to some embodiments of the present disclosure.

In some embodiments, lumen diameter and wall thickness of a dielectric barrier layer 103 is varied by use of an arrangement of a plurality of nested, telescoping tubes 103A, 103B, 103C. In a fully collapsed configuration (upper panel), each tube contributes to the electrical impedance of the dielectric barrier separating the lumen of layer 103 from discharge electrode 106. In a fully extended configuration (lower panel), fewer tubes (e.g., just one of them) from the dielectric barrier. Similarly, the lumen of layer 103 has a larger diameter of its distal-most portion in the extended configuration.

Optionally, conversion between collapsed and extended configurations is actuated by a longitudinal movement of electrical conduit 105, e.g., a distal-ward movement to move the telescoping tubes 103A, 103B, 103C into the expanded configuration. Optionally, one or more of tubes 103A, 103B, 103C has a thickness which varies along its length (i.e., from thinner to thicker), allowing a more continuous variation of dielectric barrier impedance.

Reference is now made to FIG. 1F, which schematically represents a plasma delivery tip 66 configured with a twist-adjustable lumen diameter, according to some embodiments of the present disclosure.

In some embodiments, a dielectric barrier layer 103D comprises a material (e.g., a polymer rubber) which is sufficiently compliant to allow rotation of its two ends relative to one another. The rotation distorts layer 103D, shrinking its lumenal diameter.

In some embodiments, the rotation is induced by rotation of a control tube 108 connected to a proximal end of layer 103D. Control tube 108 is itself optionally rotated from a control member positioned at a proximal end of the device. A distal end of layer 103 is anchored, for example, by attachment to sheath layer 102, which in turn is optionally fixed to a tube 104. Optionally, layer 102 is compliant enough to contract under forces exerted by the twisting of layer 103D, while still stiff enough to itself resist twisting. Tubes 104 and 108 are relatively rigid, so that deformations are concentrated within layers 102, 103D.

Reference is now made to FIG. 1G, which schematically represents a plasma delivery tip 66 configured with a twist-adjustable lumen diameter, according to some embodiments of the present disclosure.

In embodiments of the type of FIG. 1G, dielectric barrier layer 103E is formed from a helically wound sheet or ribbon of insulating material. Tightness of the windings is controlled by operation of a control member, for example, a control wire 121 anchored at anchor 120A to a first location of layer 103E, and slidably anchored at anchor 120B to a second location of layer 103E. Tightening wire 121 reduces the distance between anchors 120A, 102B, increasing winding tightness and decreasing the diameter of the lumen of layer 103E. Optionally, tension exerted on electrical conduit 105 itself controls winding.

Several windings are illustrated in FIG. 1G. Optionally, single winding is provided—that is, a long thin sheet which is rolled into a tube.

Sheath 101 is suppressed in FIG. 1G, but should be understood to be provided, for example, as shown in relation to FIGS. 1B-1F. Insulating sheath layer 102 is provided in this instance as a of hollowed-out ring, of a material which is sufficiently elastic to shrink or expand to maintain a fitted relationship with layer 103E as it shrinks or expands.

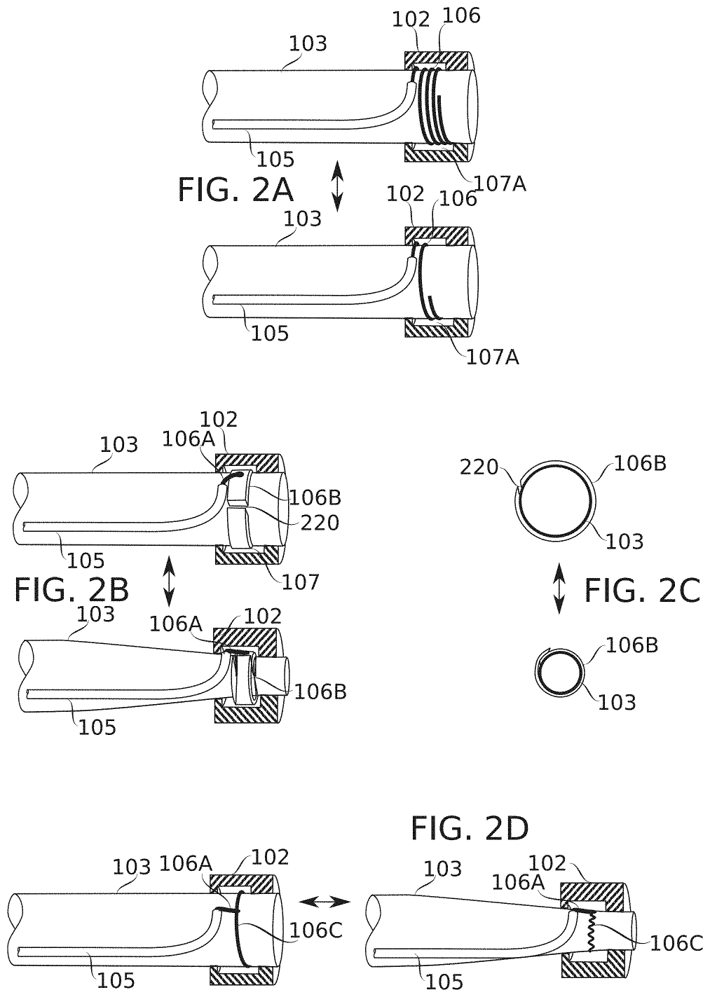

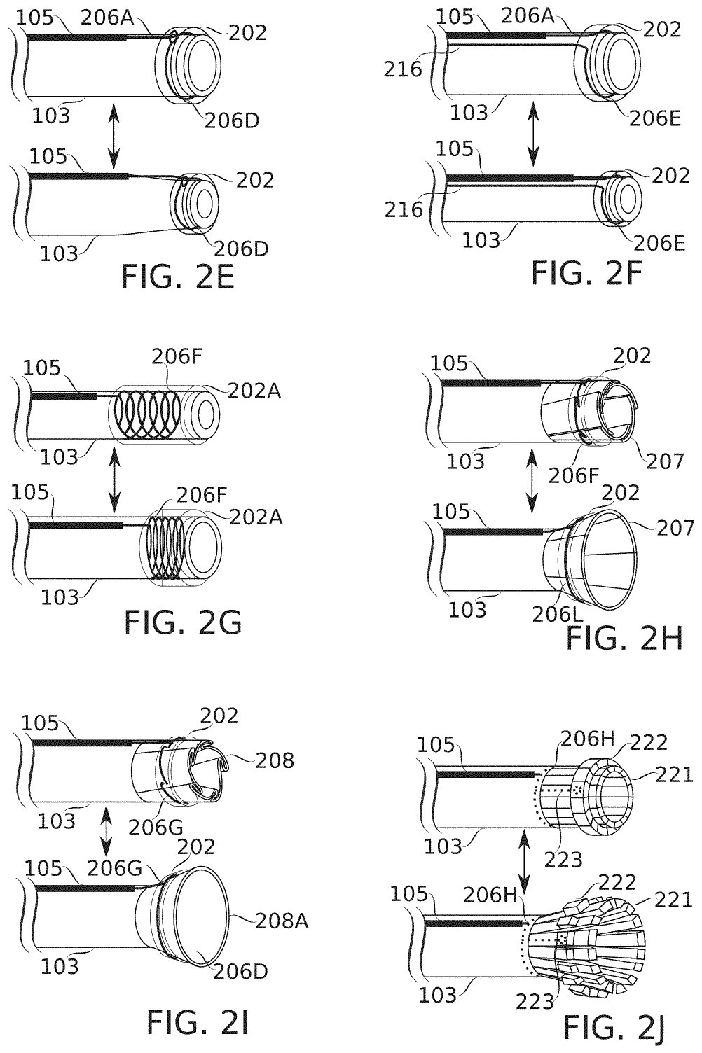

In some embodiments, an optional hollow 107 of layer 102 encapsulates electrode 106. Electrode 106 is optionally configured to adapt to changes in the diameter of layer 103E. For example, electrode 106, in some embodiments, comprises a superelastic metal in a helical shape which expands or contracts (unwinding/winding as necessary) to accommodate changes in the diameter of layer 103E. Other shape-changing electrode configurations are described, for example, in relation to FIGS. 2A-2H. In general, shape-changing electrode configurations of at least FIGS. 2A-2G are interchangeably provided to lumen- and/or wall thickness-changing embodiments of FIGS. 1B-1G, to passively accommodate lumen and/or wall thickness changes, and/or to actively control electrode geometry. In some embodiments, a proximal-to-distal extent of an electrode is altered in ratio (short divided by long) of as low as about 0.9, 0.75, 0.5, 0.25, or another ratio.

It should be understood that FIGS. 1B-1G provide examples representing a larger range of potential embodiments. In particular, embodiments of the present disclosure include devices which can widen and/or narrow the lumenal width of dielectric barrier layer 103, and/or thin and/or thicken the wall of dielectric barrier layer 103; for example by longitudinal compression, longitudinal stretching, circumferential compression, circumferential stretching, inflation/deflation, and/or rotation. Control to produce these effects is optionally exerted, for example, over wires or tubes, by self-actuating (e.g., self-expanding) properties of a material, by pressure actuation, and/or by electrical signals (e.g., electrical warming of a shape-memory metal such as nitinol to induce bending).

It is a potential advantage, in some embodiments of the present disclosure, that both electrical (isolation) and mechanical (shape-changing) functions are fulfilled by joint use of dielectric barrier layer 103, and sheath layer 102. The simplicity of their construction and mechanical operation potentially helps to keep the device diameter small (e.g., 5 mm or less), while providing sufficient adjustability to keep plasma production capacity of the plasma delivery tip 66 matched to the thermal constraints of medically safe cold plasma delivery.

Reference is now made to FIG. 1H, which schematically illustrates, in cross-section, different thermal measuring device configurations for use with a plasma delivery tip 66, according to some embodiments of the present disclosure.

Any of the plasma delivery tip 66 embodiments of the present disclosure, for example as described herein and/or as shown in other figures herein, is optionally provided with one or more sensors. Sensors 151, 152, 153 (optionally implemented, for example, as thermocouple devices, or infra-red temperature sensors) represent sensors placed at different example positions: wall-embedded sensor 151 (facing within a wall of plasma delivery tip 66), wall-embedded sensor 154 (facing to an exterior of the wall of plasma delivery tip 66), lumen-positioned sensor 152 (within plasma delivery tip lumen 22), and externally positioned sensor 153. Sensor 153A represents a sensor positioned separately from plasma delivery tip 66, for example via a separate probe 66B such as a catheter. One or more sensors is optionally provided positioned at any combination of these positions, or at another position. Any of sensors 151, 152, 153, 154 optionally comprises, for example: a temperature sensor, an electrode, a sensing fiber optic (collecting, e.g., plasm spectrum data), or another sensor; for example a sensor configured to detect the presence and/or concentration of particular ionized species (e.g., reactive oxygen and/or nitrogen species). Optionally, in some embodiments, a circuit element used in plasma generation (e.g., the discharge electrode) is also used as a sensing element. Sensing optionally comprises sensing of changes in electrical circuit properties as the electrode changes shape as a function of temperature (e.g., for embodiments comprising a shape memory alloy such as nitinol), and/or sensing changes in an electrical property such as resistivity as a function of temperature. Sensing optionally comprises measurement of impedance, e.g., to detect tissue contact and/or proximity.

Temperature sensing information is optionally provided to an operator and/or controller for use a feedback on the current temperature of the plasma delivery tip 66, and/or of plasma produced by the plasma delivery tip 66. For example, temperature sensing information is optionally returned to high voltage power controller 60 (e.g., via cable 71). Optionally, power delivery is modulated and/or switched on/off depending on sensed temperature conditions. Optionally, temperature sensing information is returned to the gas flow control unit, which modulates the gas flow and pressure based on the sensed temperature (e.g., increases pressure/flow to reduce temperature).

Optionally, temperature sensing information is used to provide feedback that guides automatic adjustments of properties of the plasma delivery tip 66 (e.g., geometric and/or electrical properties); for example, according to any of the parameter adjustment methods and/or mechanisms described herein. In some embodiments, a shape memory alloy is used to provide a plasma delivery tip 66 with self-regulating properties. For example, as a discharge electrode heats up, it is optionally configured to change to a shape which is less efficient at delivering heat-generating power. This is a potential advantage for safety.

Additionally or alternatively, an operator optionally adjusts parameters of plasma delivery based on sensed information, for example: sensed temperature (e.g., via a thermocouple), to maintain a temperature within allowed limits; and/or sensed contact and/or distance to a target, (e.g., via an electrode) to determine when plasma generation should be performed.

Spectral sensing data can be used to detect the production of spectral lines and/or detect ratios of spectral output at different wavelengths. This is optionally used to characterize plasma production. Adjustments to plasma production may be made to achieve a targeted spectral profile.

Sensors 152, 153, in some embodiments, are positioned within the plasma stream as it exits lumen 22 (sensor 152), or after it exits lumen 22 (sensor 153). Optionally, a sensor 152, 153 is variably positioned, for example by advancing or retracting cables 152A, 153A which connect them to a measurement recorder positioned on proximal to the plasma delivery tip 66 (e.g., positioned outside a body into which plasma delivery tip 66 is inserted).

Sensors 152, 153 optionally comprise temperature sensors, placed where they measure plasma temperature by direct thermal contact. Optionally, thermal sensing (e.g., IR-based thermal sensing) is performed from outside the plasma stream, e.g., using a sensor 151, 154 comprising a thermocouple embedded in the wall of plasma delivery tip 66 (and transmitting measurement information along cable 151A, 154A).

In some embodiments, measurement readings from a sensor 151, positioned within a wall of plasma delivery tip 66, are indirect indications of plasma temperature, insofar as measurements are also potentially affected by the thermal absorbing and/or thermal conducting properties of the wall in the presence of plasma. Optionally, readings are calibrated to previously measured corresponding plasma temperatures, e.g., according to equilibrium temperature and/or according to rates of temperature change. In some embodiments a sensor (e.g., sensor 154) comprises a temperature sensor such as a thermocouple which is placed where it primarily indicates temperature outside the plasma delivery tip and/or plasma plume itself. Sensor 154 is shown exposed on an outer wall of plasma delivery tip 66. Optionally, an external sensor is brought into a working position to detect temperature of and/or in the vicinity of plasma delivery tip from another probe.

Optionally, any of sensors 151, 152, 153, 154 comprises a contact and/or proximity sensor. Optionally, the sensor comprises an electrode, and sensing of contact and/or proximity comprises detecting changes in impedance (e.g., resistance) experienced as the electrode moves in its environment along with plasma delivery tip 66.

Reference is now made to FIG. 2A, which schematically represents a plasma delivery tip 66 configured with an adjustable-length plasma discharge electrode 106, according to some embodiments of the present disclosure.

In some embodiments, discharge electrode 106 comprises a wire (e.g., a superelastic wire) which can be relatively advanced from (upper panel) and/or retracted into (lower panel) a shielded portion of electrical conduit 105, with hollow 107A of insulating sheath layer 102 being sized to accommodate more or fewer windings. In some embodiments, dielectric barrier 103 is shape-changing (e.g., changing in lumen diameter according to any of the embodiments shown and/or described in relation to FIGS. 1B-1G), and the winding radius of electrode 106 increases or decreases to accommodate such changes.

Increasing the number of windings of electrode 106 also changes the effective longitudinal length of electrode 106. Optionally, this is used to adjust plasma generating properties of the plasma delivery tip 66, wherein a longer electrode potentially generates more and/or more concentrated plasma than a shorter electrode. The increase in plasma generation potentially comes with a tradeoff of increased heating, so a shorter electrode may be preferred in some cold plasma delivery scenarios.

Reference is now made to FIG. 2B, which schematically represents a plasma delivery tip 66 configured with an adjustable-diameter plasma discharge electrode 106B, according to some embodiments of the present disclosure. Reference is also made to FIG. 2C, which schematically represents an end-on view of the adjustable-diameter plasma discharge electrode 106B of FIG. 2B, according to some embodiments of the present disclosure.

Electrode 106B is configured as a band of electrically conductive material shaped into a ring with a cut 220. Cut 220 is optionally diagonal; for example as shown (e.g., oblique relative to a radial direction) and/or oblique to a longitudinal axis extending across electrode 106B. Conductor 106A (e.g., a central conductor of electrical conduit 105) interconnects electrode 106B with a power supply. When dielectric barrier layer 103 has a relatively large lumen diameter (upper panels, e.g., as described in relation to FIGS. 1B-1G), electrode 106B assumes a correspondingly expanded configuration. When dielectric barrier layer 103 has a relatively smaller lumen diameter (lower panels), electrode 106B assumes a more self-overlapping configuration. Optionally, electrode 106B is partially self-overlapping even in its most expanded state. The gap in electrode 106B, and/or overlaps in electrode 106B potentially introduce some asymmetry in the shape of the generated plasma plume. Overlap is optionally in the same plane as the ring, and/or overlap along a longitudinal axis.

Reference is now made to FIG. 2D, which schematically represents a plasma delivery tip 66 configured with an adjustable-diameter plasma discharge electrode 106C, according to some embodiments of the present disclosure.

Electrode 106C is shaped with a wave and/or zig-zag pattern, assumed when dielectric barrier layer 103 is at a relatively smaller diameter. As layer 103 expands, the waves/zig-sags straighten, allowing the diameter of electrode 106C to also expand. Optionally, electrode 106C is formed from a superelastic material such as nitinol. Optionally, electrode 106C comprises a conductive material printed, painted or otherwise deposited onto an elastic supporting substrate.

Reference is now made to FIG. 2E, which schematically represents a plasma delivery tip 66 configured with a lasso-type adjustable-diameter plasma discharge electrode 206D, according to some embodiments of the present disclosure. Reference is also made to FIG. 2F, which schematically represents a plasma delivery tip 66 configured with an open loop-type adjustable-diameter plasma discharge electrode 206E, according to some embodiments of the present disclosure.

In some embodiments, either of electrodes 206D, 206E can be tightened (shrinking their diameter) by manipulating (e.g., pulling) a tensioning member 206A. Additionally or alternatively, electrode 206E can be tightened by manipulating returning loop-back electrode portion 216. In some embodiments, loop-back portion 216 goes all the way back to a control member. In some embodiments, loop-back portion 216 is anchored at some location, e.g., to the side of the plasma delivery tip 66.

Optionally, tensioning member 206A is also a conductor of electrical conduit 105. Shrinking electrode 206D, 206E also compresses dielectric barrier layer 103, and correspondingly, in some embodiments, reduces a lumenal diameter of layer 103. Optionally, relaxing the loop of electrode 206D, 206E allows layer 103 (and its lumenal diameter) to expand.

The electrode 206E, in some embodiments, extends around at least 75% of a circumference of dielectric barrier layer 103. It is noted that radial asymmetry of any discharge electrode potentially induces a corresponding asymmetry in a plasma plume produced by a plasma delivery tip.

Insulating layer 202 is optionally implemented as a ring gasket enclosing electrode 206D, 206E. Optionally, insulating layer 202 is otherwise embodied (e.g., as a tube within which electrode 206D, 206E is at least partially embedded); for example, according to any of the configurations shown and/or discussed in relation to FIGS. 1B-1G.

Reference is now made to FIG. 2G, which schematically represents a plasma delivery tip 66 configured with a helix-type adjustable-length plasma discharge electrode 206F, according to some embodiments of the present disclosure.

Optionally, the helix of plasma discharge electrode 206F passively expands and contracts longitudinally as a result of longitudinal expansion/contraction of layer 202A and/or layer 103, for example when implemented by one of the configurations of FIGS. 1B-1G. Optionally, a longitudinal size of electrode 206F is directly controlled, e.g., expanded by pulling on a member 206A, which may be a central conductor of electrical conduit 105. In some embodiments, expansion is controlled by heating (e.g., of a superelastic alloy shaped to expand when crossing its transition temperature). In some embodiments, expansion is controlled by use of a magnetic field, optionally a magnetic field induced externally to the plasma delivery tip.

Reference is now made to FIGS. 2H-2J, which schematically represent a plasma delivery tip 66 configured with a segmented expanding distal end, according to some embodiments of the present disclosure.

In some embodiments, a distal end of a plasma delivery tip 66 is configured with a circumferential arrangement of lamellar portions 207, 208, 221, which are configured to interconvert between a collapsed configuration (upper panels) and an expanded configuration (upper panel) in which the lamellar portions 207, 208 221 flare radially outward. The lamellar portions are optionally self-expanding (e.g., they are elastically predisposed to expand upon exiting a confining sheath, not shown), expanded by loosening of their electrode (e.g., as descried in relation to FIGS. 2E-2F and/or 3B), and/or expandable by another method.

In some embodiments the lamellar portions comprise portions of a folded-up element 208A (FIG. 2I, upper panel) which expands to a circumferentially unbroken shape (lower panel). FIG. 2H illustrates lamellar portions 207 which initially overlap, and expand to a “barrel stave” adjacent configuration, the expansion being optionally limited by the expansion diameter of discharge electrode 206L. Optionally, the expanded configuration of lamellar portions 207 keeps a small amount of overlap, helping to maintain an unbroken circumference. In some embodiments, (FIG. 2J), the lamellar portions 221 expand to leave gaps between them. Although this potentially allows plasma supply gas to escape to through the sides, the gaps may be small enough that under laminar flow conditions, side-escaping plasma is negligible. Optionally, gas escape is prevented by webbing between the lamellar portions 221, and/or an expanding lining of the lumen of the plasma delivery tip 66.

The discharge electrode 206L, 206G (FIGS. 2H-2I) is optionally itself circumferential in shape when expanded, the circumference collapsing as necessary accommodate the collapsed configuration of lamellar portions 207, 208. In the collapsed configuration, electrode 207, 208 is still potentially operable, although the expanded configuration (typically the configuration which can provide the most and/or most concentrated plasma without over-heating) potentially provides a more uniform and/or predictable plasma plume. The dielectric properties of lamellar portions 207, 208 help determine discharge characteristics which affect plasma production within the lumen. Outer insulation of discharge electrode 207, 208 is optionally provided by expanding rings 202, or an expanding tube. Optionally, discharge electrode 207, 208 is embedded within the material of lamellar portions 207, 208, so as to be electrically insulated from all sides.

Optionally, discharge electrode 206H comprises projections 223 (FIG. 2J, only one projection shown) which extend into a plurality of the lamellar portions 221. Optionally, the projections 223 comprise terminal expansions, opposite of which most plasma generation occurs. Outer electrical insulation is optionally provided as individual expansions 222, as a ring, a tube, or by embedding projections 223 within the material of lamellar portions 221. Optionally, the embodiment of FIG. 2J is provided with a ring electrode interconnecting the lamellar portions 221, e.g., as a ring which collapses (like electrode 206G, for example), or as a plurality of electrodes embedded in the lamellar portions 221 which are interconnected by a circumferential wire.

### Steering and Tip Shape Options

Reference is now made to FIG. 3A, which schematically represents a plasma delivery tip 66 configured with a steerable end, according to some embodiments of the present disclosure.

In some embodiments, electrical conduit 105 and/or a conductor and/or cable portion of electrical conduit 105 is slidable relative to inner dielectric barrier layer 103, but connected to a distal end of layer 103, so that, e.g., tension exerted on electrical conduit 105 or a portion thereof causes layer 103 to bend. Optionally discharge electrode 206J comprises, for example, a circular wire, or has another electrode design such as one of those described herein; for example, a mesh, helix, and/or split band. Electrode 206J is optionally insulated by an outer insulating layer 202.

Reference is now made to FIG. 3B, which schematically represents a plasma delivery tip 66 configured with an end configured to constrict to a penetrating cone, according to some embodiments of the present disclosure.

In some embodiments, electrode 206K is configured with a noose or other constricting electrode design, which can be tightened enough to narrow a distal end 204 of dielectric barrier layer 203A down to a pointed tip which can be used, for example, to penetrate resistance and/or help guide forward navigation. Optionally, the point reduces a distal aperture of layer 203A down to less than its unconstricted diameter; e.g., less than 50% or less than 25% of its unconstricted diameter. Optionally, distal end 204 is beveled around its circumference (conically beveled) to reduce the amount of material that is gathered together when electrode 206K is tightened. Additionally or alternatively, material near the tip is stretched to help sharpen the point.

Reference is now made to FIG. 4, which schematically represents a plasma delivery tip 66 configured with a beveled distal end, according to some embodiments of the present disclosure.

In some embodiments, oblique slice bevel 408 optionally helps the plasma delivery tip 66 to penetrate resistance, and/or helps guide forward navigation. Within the plasma delivery tip 66, any of the electrode and insulating designs already described with respect to a blunt-ended plasma delivery tip 66 are optionally provided; for example, electrical conduit 105, discharge electrode 106, space 107, sheath layer 102, and/or dielectric barrier layer 103.

Optionally, tubular portions proximal to the plasma delivery tip 66 are provided with mesh reinforcement 411 and/or coil reinforcement 412, which act as stiffeners. This potentially allows the plasma delivery tube comprising the plasma delivery tip 66 to act as its own guide when navigating to an intrabody site to which plasma is to be delivered. Such stiffeners are optionally provided to assist navigation of any of the plasma delivery tips described herein. It should be understood in particular that the steering configuration of FIG. 3A is optionally also provided to embodiments with the features of FIG. 4.

Reference is now made to FIG. 5A, which schematically represents a plasma delivery tip 66 configured with a channeled insulating tube 502, according to some embodiments of the present disclosure. Reference is also made to FIG. 5B, which schematically represents a plasma delivery tip 66 configured with a helically channeled insulating tube 502B, according to some embodiments of the present disclosure. Further reference is made to FIG. 5C, which schematically represents an end-on view of a cross section of the channeled insulating tube 502, 502B of FIGS. 5A-5B, according to some embodiments of the present disclosure. Additional reference is made to FIG. 5D, which schematically represents a plasma delivery tip 66 configured with a helically channeled insulating tube 502C, according to some embodiments of the present disclosure.

In all of the examples of FIGS. 5A-5D, an outer surface of an outer insulating layer 502, 502B, 502C is shaped with one or more straight (layer 502, FIG. 5A) or helical (layers 502B, 502C, FIGS. 5B, 5D) channels 510, 511, 512. FIG. 5B shows a cross section that produces such channels, including a plurality of circumferentially arranged projections 521, separated by indentations 522. Channels are optionally bordered by a sheath tube 101. Optionally, channels are formed as tubes passing longitudinally through an insulating layer. Circumferential locations of dielectric barrier layer 103 and discharge electrode 106 are also shown.

When plasma plume 10 introduces gas, e.g., into a confined intrabody space, there is potentially a buildup of gas volume and/or pressure. Channels 511, 512, 513 are optionally configured to relieve this buildup, gas buildup relief may be passive (i.e., a proximal end of a tube with which channels 511, 512, 513 are in communication is open to ambient pressure), and/or active (for example, a suction pump is optionally applied to a proximal end of a tube with which channels 511, 512, 513 are in communication).

Gas which has interacted with the environment is potentially cooled down by this, so that when it returns, it can absorb some heat from the plasma delivery tip 66. Return by a helical channel has a potential advantage to increase this counter-cooling effect, e.g., by increasing the path length along which heat can be absorbed. Optionally, a coolant fluid (e.g., a gas or liquid) is delivered through one or more of the channels 511, 512, 513. Optionally, the coolant fluid is returned through another one or more of the channels 511, 512, 513.

A potential issue when ionization gas is being transported back along the outside of insulating layer 502, 502B, 502C is that it may itself be subject to electrical fields at or near its breakdown voltage, resulting in ectopic plasma production by induction. In some embodiments, the insulating layer itself is made of thick enough dielectric material to ensure that this is prevented. In some embodiments, a secondary gas is mixed with the ionization gas in the region of plasma generation, raising the breakdown voltage of gas before it is exhausted.

### Gas-Surrounded Discharge Electrode Configurations

Reference is now made to FIG. 6A, which schematically illustrates a plasma delivery tip 66 comprising a discharge electrode assembly 601 which is positioned within a lumen of a gas supply tube 603, according to some embodiments of the present disclosure. Reference is also made to FIG. 6B, which schematically illustrates position adjustments of the discharge electrode 606 within a plasma delivery tip 66, according to some embodiments of the present disclosure.

In some embodiments, discharge electrode assembly 601 comprises a discharge electrode 606 encapsulated within a dielectric barrier layer 602, and sized to reside within a lumen 610 of a gas supply tube 603. Discharge electrode assembly 601 is coupled to a voltage supply via coaxial cable 605 (and/or another electrical conduit), allowing a plasma-generating voltage field to be established within a gas flow 8 through a lumen of gas supply tube 603. Ionization generated within gas flow 8 generates plasma which exits lumen 610 as plasma plume 10.

A potential advantage of this design is that it is particularly suited for small-diameter probes, insofar as it allows production of devices with fewer functional layers required. Optionally, the design allows conversion of a lumen (e.g., working channel) of an existing devices into a gas supply tube, by insertion of discharge electrode assembly 601, its cabling, and any optional positioning supports (e.g., as described in relation to FIGS. 6C-6G); and by attachment to an ionization gas source.

Control of plasma parameters such as plasma temperature is optionally performed by controlling ionization gas flow rate, pulsing ionization gas flow, and/or dynamically mixing ionization gas flow with other gases to control ionizing susceptibility and/or atomic mass.

In some embodiments (FIG. 6B), discharge electrode assembly 601 is moveable within gas supply tube 603. Movement is optionally along a longitudinal axis (e.g., upper and lower panels of FIG. 6B), and/or radially (right panel of FIG. 6B). These movements are optionally performed to adjust plasma generation, plasma plume characteristics, and/or thermal transfer characteristics. For example, movements of discharge electrode assembly 601 to different depths within gas supply tube 603 potentially allow locating a position wherein plasma generation efficiency, temperature, and plasma plume length are optimal for a current target and/or target position. Movements of the discharge electrode assembly 601 to different depths also potentially allow adjusting distance to a targeted surface.

Extruding discharge electrode assembly 601 from gas supply tube 603 potentially places it in a region of reduced plasma supply gas concentration, but may allow greater selectivity of the intrabody site targeted for plasma delivery.

Moving discharge electrode assembly 601 to more radially offset positions within lumen 610 within gas supply tube 603 potentially affects the intensity, shape and/or position of the generated plasma plume 610.

Reference is now made to FIGS. 6C-6D, which schematically illustrate a positioning support 621 configured for use with a plasma delivery tip 66 comprising a discharge electrode assembly 601 which is positioned within a lumen of a gas supply tube 603, according to some embodiments of the present disclosure. FIG. 6C illustrates a blunt-ended gas supply tube 603, while FIG. 6D illustrates a pointed-end (beveled) gas supply tube 603B, optionally used as a needle or trocar tip for penetrating tissue.

In some embodiments, positioning support 621 comprises a vented disc to which discharge electrode assembly 601 is mounted. For example, an electrical conduit 605 supporting discharge electrode assembly 601 itself passes through a center of positioning support 621, thereby helping to maintain a centered position for discharge electrode assembly 601 while allowing discharge electrode assembly 601 to be moved longitudinally along lumen 610.

Positioning support 621 is vented to allow supply gas to pass through and/or over it. The flow of gas is in a distal direction during plasma delivery. Optionally, a direction of gas flow is reversed to remove gas (e.g., by suction) from a working area. During reverse flow, plasma generation is optionally halted. In some embodiments, delivery of plasma alternates with removal of gas several times during a plasma delivery treatment of a region. Venting of positioning support 621 comprises, e.g., indentations around the circumference of positioning support 621, and/or perforations.

Optional vents 622 of FIG. 6C (implemented as holes) and/or optional vents 623 of FIG. 6E (implemented as slots) may be provided as an additional or alternative feature to allow venting of ionization gas. This is a potential advantage to allow the plasma delivery tip to be pressed up against a target surface to be treated, without pressure instabilities and/or contact interruptions due to the build-up and uncontrolled release of ionization gas during plasma generation. Optionally, venting (e.g., using shapes like those of vents 622, 623, or another shape) is provided to any of the plasma delivery tip embodiments described herein (e.g., any of FIGS. 1B-1G, and/or 2A-2J).

Reference is now made to FIGS. 6E-6G, which schematically illustrate positioning support allowing both longitudinal′ and radial position adjustment of a plasma delivery tip 66 comprising a discharge electrode assembly 601 which is positioned within a lumen of a gas supply tube 603, according to some embodiments of the present disclosure.

In some embodiments, positioning support for discharge electrode assembly 601 comprises a rotating position support 631, and a slotted position support 632. Discharge electrode assembly 601 is mounted to a member (e.g., coaxial cable 605) extending longitudinally within lumen 610 of gas supply tube 603. Coaxial cable 605 crosses through rotating position support 631 at a location 641 (e.g., a hole) radially offset from the center of lumen 610. It also passes through slotted position support 632, at a slot 642 which radially crosses lumen 610.

Upon rotation of coaxial cable 605, rotating position support 631 also rotates. This causes movement of coaxial cable 605 (at the point of crossing) along a circular path. Distal to this, slot 642 constrains coaxial cable 605 so that it is re-centered along one axis, but remains free to move back and forth along a substantially orthogonal axis. As a result, discharge electrode assembly 601 is constrained to move along a narrow oval and/or substantially linear path upon rotation of coaxial cable 605, to which it is mounted.

The amount of “wobble” (bending and/or translation movement along the constrained axis) in the motion of discharge electrode assembly 601 induced by the offset of location 641 from slot 642 is optionally set by adjustment of the relative distances of discharge electrode assembly 601 and rotating position support 631 from slotted position support 632. Optionally, a thickness of slotted position support 632 along a proximal-to-distal axis of lumen 610 is made greater to reduce and/or prevent wobble.

Optionally, slotted position support 632 is fixed within lumen 610 rotationally and/or along a proximal-distal axis of lumen 610; e.g., by shape-interference and/or friction with the walls of gas supply tube 603.

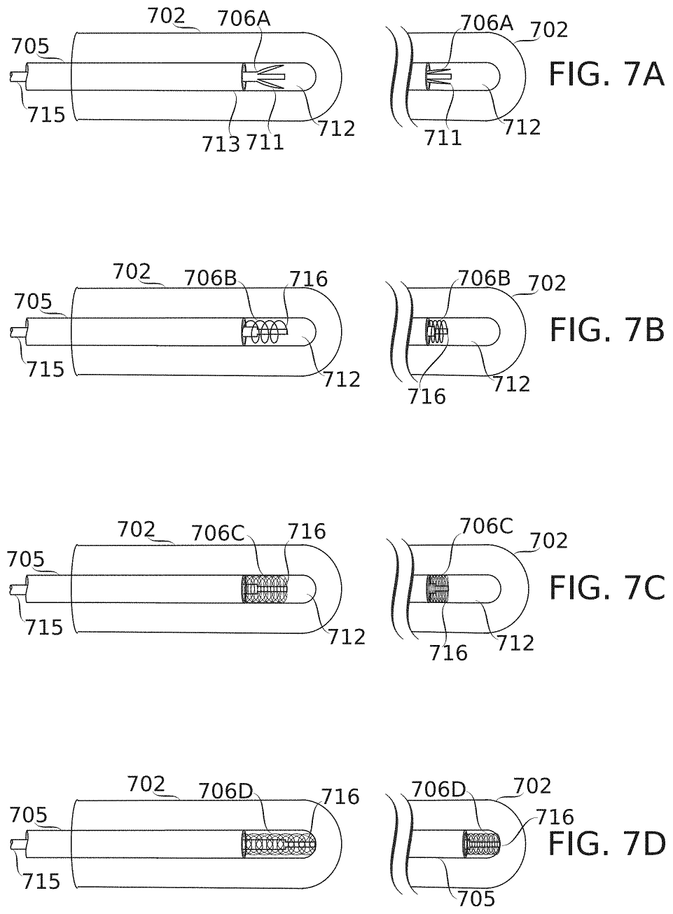

Reference is now made to FIGS. 7A-7D, which schematically illustrate adjustable discharge electrodes 706A, 706B, 706C, 706D of various discharge electrode assemblies configured to be positioned within a lumen of a gas supply tube, according to some embodiments of the present disclosure.

Adjustable discharge electrode 706A (FIG. 7A) comprises a plurality of tines 711 which can be actuated to expand or contract within a capsule space 712 formed within dielectric barrier layer 702. Actuation comprises, for example, longitudinal movements of a cable 715 (which may also be a center conductor of a coaxial cable 705), to move the tines in and out of a constraining overtube 713 (which may comprise, for example, one or more surrounding layers of coaxial cable 705). Optionally, tines 711 are formed of a superelastic material, for example, nitinol.

Additionally or alternatively, in some embodiments, tines 711 are self-actuating as a function of temperature. For example, tines 711 optionally comprise a shape memory alloy (nitinol, for example) with a transition temperature set to change the shape of discharge electrode 706A to a less-efficient plasma generating configuration when hot. This provides a potential advantage for reducing or preventing delivery of overheated plasma to tissue. Shape-memory effects are optionally used to provide thermal self-actuation of other electrode shapes, for example, any of electrodes 706B, 706C, 706D.

In some embodiments, thermally actuated shape changes (e.g., of a discharge electrode or other discharge circuit element) are used to provide temperature monitoring; for example, by measurement of changes in electrical circuit properties induced by shape changes.

Adjustable discharge electrode 706B (FIG. 7B) comprises a helix (e.g., a helical spring). Optionally, a length of discharge electrode 706B is adjusted within capsule space 712 by longitudinal movements along a proximal-distal axis of a control member 716 attached to discharge electrode 706B.

Adjustable discharge electrodes 706C, 706D (FIGS. 7C-7D) each comprise a collapsible mesh. Optionally, a length of discharge electrode 706C is adjusted within capsule space 712 by longitudinal movements along a proximal-distal axis of a control member 716 attached to discharge electrode 706C, 706D. In the embodiment of FIG. 7D, adjustment also moves dielectric discharge barrier 702 relative to coaxial cable 705. Optionally, this allows discharge electrode 706D to effectively fill capsule space 712 at any size.

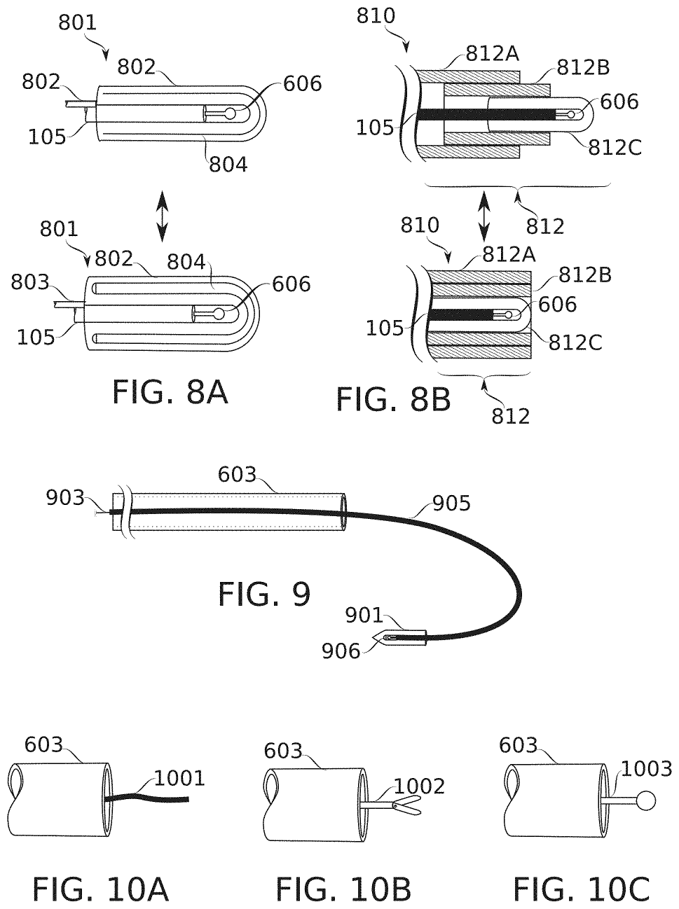

Reference is now made to FIG. 8A, which schematically illustrates a discharge electrode assembly 801 configured to be positioned within a lumen of a plasma gas supply tube, and comprising a dielectric barrier layer 802 adjustable by inflation, according to some embodiments of the present disclosure. Discharge electrode assembly 801 represents particular embodiments of a discharge electrode assembly 601, and is optionally provided with any suitable plasma gas supply tube, discharge electrode, and/or position support for example as described in relation to FIGS. 6A-7D, herein.

In some embodiments, dielectric barrier layer 802 is inflatable to an expanded configuration (lower panel) from a collapsed configuration (upper panel); for example, by the injection of a fluid (gas or liquid) through inflation tube 803 to expand inflatable lumen 804. Optionally, inflation is performed by exerting axial compression on the dielectric barrier. Inflation changes the dielectric barrier properties of dielectric barrier layer 802, changing plasma generation and/or plasma plume properties (e.g., a thicker barrier reduces plasma generation and/or increases plasma breakdown voltage). In some embodiments, inflation is triggered by heating of a volatile liquid within lumen 804 (optionally, without use of an inflation tube 803), providing a feedback mechanism which reduces power dissipation in response to elevated temperatures. This has a potential advantage for device safety. Optionally, another temperature-sensitive transition of material properties is used for adjusting the dielectric barrier layer 802 is provided, for example, a solid which melts to a fluid, or a gas which expands. Optionally, a shape-memory alloy or polymer with a temperature-dependent expanding or shrinking shape is placed within lumen 804 to change its shape.

Reference is now made to FIG. 8B, which schematically illustrates a discharge electrode assembly 810 configured to be positioned within a lumen of a plasma gas supply tube, and comprising a multi-laminar dielectric barrier layer 812, according to some embodiments of the present disclosure.

Adjustable discharge electrode assembly 810 represents particular embodiments of a discharge electrode assembly 601, and is optionally provided with any suitable plasma gas supply tube, discharge electrode, and/or position support for example as described in relation to FIGS. 6A-7D, herein.

In some embodiments, dielectric barrier layer 812 comprises a plurality of layers 812A, 812B, 812C configured to slide past each other to longitudinally expand or contract discharge electrode assembly 810. In the expanded configuration (upper panel), discharge electrode 606 is surrounded by a relatively thin dielectric barrier comprising, e.g., just lamina 812C. In the collapsed configuration (lower panel), discharge electrode 606 is surrounded by a relatively thick dielectric barrier comprising, e.g., laminae 812A, 812B, and 812C. Changing total dielectric barrier thickness changes the dielectric barrier properties of dielectric barrier layer 812, potentially changing plasma generation and/or plasma plume properties (e.g., a thicker barrier reduces plasma generation).

Actuation is optionally performed, for example, by application of pressure to longitudinally advance electrical conduit 105 and/or a portion thereof (for example, a conductor connected to electrode 606) while lamina 812A is held restrained (e.g., anchored to a wall of a gas delivery tube, and/or anchored to an outer layer of electrical conduit 105). Optionally, electrical conduit 105 and/or the lamina 812A, 812B, 812C are provided with mechanical stops to prevent over-withdrawal and/or over-extension.

Reference is now made to FIG. 9, which schematically illustrates a discharge electrode assembly 601 and electrical conduit 905 (an instance of an electrical conduit 105) configured for use as a guidewire for guiding advance of gas supply tube 603, according to some embodiments of the present disclosure. Apart from its function as a shielded transmitter of electrical power, electrical conduit 905 is optionally constructed mechanically to act as a guidewire of gas supply tube 603; the two together comprising elements of a catheter system. Optionally, a standard catheter guidewire with an otherwise hollow core is provided with a conductive wire 903 extending along the hollow core (with additional electrical insulation if necessary) to form a combined guidewire/electrical power transmission line. Optionally, the conductive wire 903 is also a control wire, operable, for example, to actuate steering at a distal tip of electrical conduit 905.

Optionally, a discharge electrode and/or dielectric barrier layer of discharge electrode assembly 901 is configured as any of the discharge electrode assemblies described herein; for example in FIGS. 6A-7D. Optionally or additionally, a tip of discharge electrode assembly 901 is tapered and/or pointed, potentially assisting in forward navigation. Optionally the tip of the electrode is rounded, to provide an atraumatic tip to advance inside body lumens.

Optionally, any portion of discharge electrode assembly 901 is provided with a radiopaque marker material. Optionally, electrode 906 of a discharge electrode assembly 901 comprises a conducting material (e.g., gold, silver, and/or platinum) which is also a radiopaque material.

Reference is now made to FIGS. 10A-10C, which schematically represent the optional use of alternative medical tools 1001, 1102, 1003 with a gas supply tube 603, according to some embodiments of the present disclosure. The medical tool used, in some embodiments, comprises a longitudinally extended member long enough to extend from a proximal side of the gas supply tube 603 to a distal side of the gas supply tube, and thin enough to insert within the gas supply tube 603. In some embodiments, the medical tool terminates in a device which operates to cut, dissect, penetrate, ablate, sample, or otherwise manipulated tissue positioned near the distal side of the gas supply tube. In some embodiments, the medical tool terminates in an atraumatic tip (e.g., of a guidewire), optionally tapered to assist advance of the tool into an aperture of a body lumen.

In some embodiments, a gas supply tube 603 configured for the supply of an ionization gas converted to a plasma by a discharge electrode assembly 601 is optionally convertible in place to a working channel by removing its discharge electrode assembly 601, and inserting another medical tool instead. Examples of such medical tools include guide wire 1001, cutter 1002, and ablation electrode 1003.

In some embodiments, a discharge electrode 106 extends circumferentially around a gas delivery lumen of gas supply tube 603 (e.g., as described in relation to FIGS. 1B-4), leaving the lumen of gas supply tube 603 available for use as a working channel by insertion of a tool such as guide wire 1001, cutter 10002, and/or ablation electrode 1003.

Potential advantages of this configuration include reducing swapping of tubes during a procedure which uses multiple tools, and/or allowing the gas supply tube 603 itself to be the largest-diameter tube in a procedure (which is a potential advantage for reducing power density).

Discharge Electrodes Separate from Gas Delivery Lumens

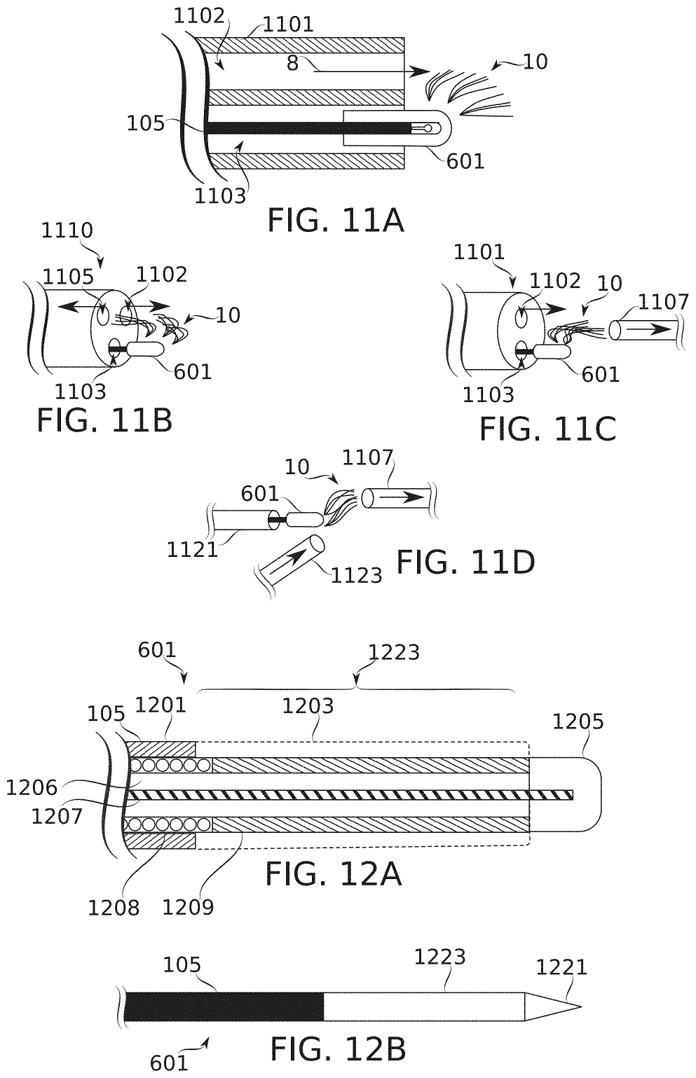

Reference is now made to FIGS. 11A-11D, which schematically illustrate different arrangements of lumens for ionization gas delivery, plasma/ionization gas removal, and/or current delivery, according to some embodiments of the present disclosure.

FIG. 6A (for example) illustrates a configuration wherein a flow 8 of ionization gas is delivered through a same lumen 610 which is used to deliver a discharge electrode assembly 601. As shown, for example, in FIGS. 11A-11D, ionization gas delivery is optionally through a lumen separate from one which delivers discharge electrode assembly 601.

In FIG. 11A, a plasma device tip 1101 comprises a gas delivery lumen 1102, and a working channel 1103 configured to allow passage discharge electrode assembly 601 to its end. Plasma plume 10 is generated outside of plasma device tip 1101, where flow 8 of ionization gas comes into proximity with discharge electrode assembly 601. Optionally, plasma device tip 1101 comprises a distal extension (not shown) in which flow 8 remains confined, and into which discharge electrode assembly 601 can also be advanced. Optionally, lumen 1102 serves alternately to provide ionization gas and to remove gas from the working site. Optionally, lumen 1103 itself is configured to deliver and/or remove ionization gas. Optionally, its lumen is larger than a diameter of discharge electrode assembly 601, and/or discharge electrode assembly 601 is sufficiently extruded from lumen 1103 during operation as to allow gas to pass into/out of lumen 1103.

Optionally (FIG. 11C), plasma and/or ionization gas are scavenged by suction from another lumen 1107 introduced into the region of plasma delivery. Additionally or alternatively (FIG. 11B), a plasma device tip 1110 comprises a plasma/gas scavenging lumen 1105 in addition to gas delivery lumen 11102, and working channel 1103.

In some embodiments, all three lumens are provided on separate probes; e.g., discharge electrode assembly 601 is delivered through lumen 1121, ionization gas is delivered through lumen 1123, and plasma/ionization gas is scavenged through lumen 1107.

A potential advantage of generating plasma outside of a confining lumen is reduction of hot-spot build-up. Optionally, plasma flow can be directed with more flexibility; moreover, moving the flow of ionization gas around also potentially results in a reduction of hot-spot build up. Optionally, the plasma plume can be generated with a diameter larger than the inner diameter of the lumen which supplies the ionization gas.

Another potential advantage is to allow generating plasma inside cavities too small for introduction of a whole plasma delivery tip including delivery lumen, but large enough to allow introduction of the electrode assembly and gas flow.

### Small-Diameter Discharge Electrode Assemblies

Reference is now made to FIGS. 12A-12B, which schematically illustrates construction details of a small-diameter discharge electrode assembly 601, according to some embodiments of the present disclosure.

In some embodiments, an outer diameter of discharge electrode assembly 601 (e.g., one suitable for use in relation to flow-surrounded discharge electrode embodiments like those of FIGS. 6A-6G) is constructed as distal extension of an electrical conduit 105 comprising a coaxial cable, in such a way as to minimize its maximum diameter to about the diameter of the electrical conduit 105 itself. This provides a potential advantage for building a complete plasma delivery tip 66 with a small size.

In some embodiments, electrical conduit 105 comprises an outer insulator 1201, an electrical shield layer 1208, an inner insulator 1206, and a core conductor 1207. To form the discharge electrode assembly 601, the outer insulator 1201 and electrical shield layer 1208 are stripped away from inner insulator 1206. Over a portion 1223 of the stripped-away region, rigid or semi-rigid tube 1209 replaces electrical shield layer 1028. Tube 1209 optionally comprises a solid metal (e.g., stainless steel) tube, a wire-coil construction, or another rigid or semi-rigid construction. Over tube 1209 (optionally) is extended an outer insulator 1203 (which may optionally even be the original outer insulator 1201, returned into place, for example). Outer insulator 1203 is optional, since the electrical potential of rigid tube 1209 is optionally set to the same ground potential of the body in which discharge electrode assembly 601 is to be operated.

A small portion of core conductor 1207 extends distally beyond region 1223. This portion is encapsulated with dielectric barrier layer 1205, across which voltage is delivered to convert ionization gas into cold plasma. Optionally, dielectric barrier layer 1205 is blunt, or instead (FIG. 12B), a pointed dielectric barrier layer 1221 is provided. The pointed version provides a potential advantages for using discharge electrode assembly 601 as the tip of a guide wire, for example as described in relation to FIG. 9. Optionally, the pointed version of FIG. 12B is useful for penetrating tissue to access a target region.

### Side-Discharging Plasma Delivery Tips

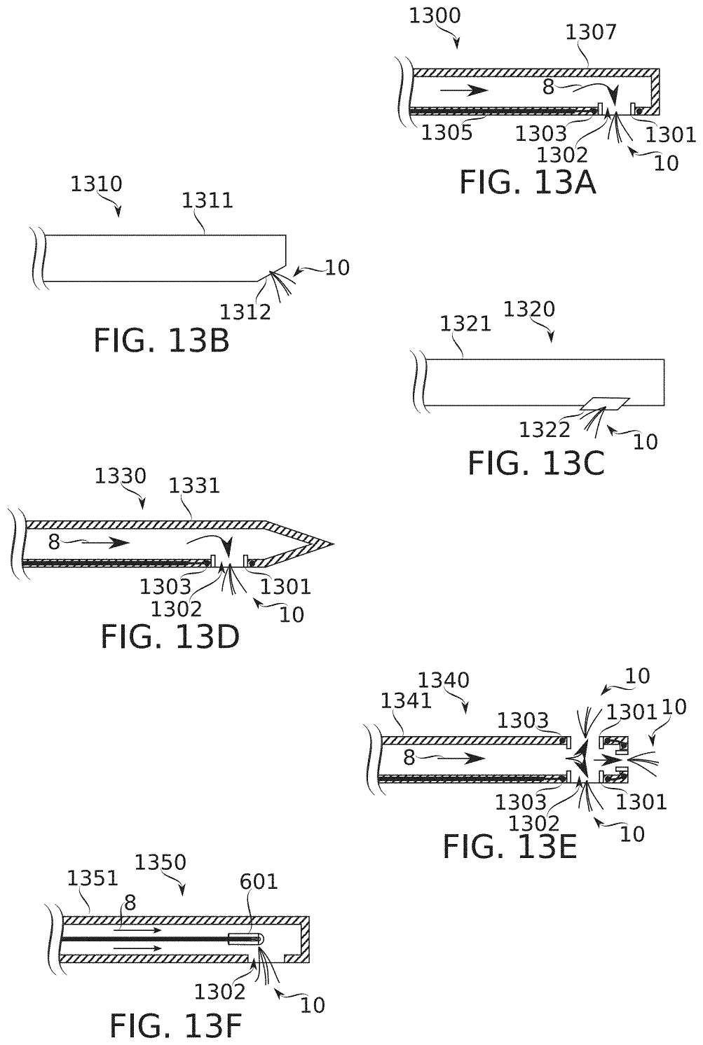

Reference is now made to FIGS. 13A-13F, which schematically illustrate plasma delivery tips 66 configured to generate oblique- and/or perpendicular-angle plasma plumes relative to a longitudinal axis of the plasma delivery tip 66, according to some embodiments of the present disclosure.

In some embodiments, a flow 8 of ionization gas is directed at least partially to a lateral side of a plasma delivery tip 1300, 1310, 1320, 1330, 1340, 1350, producing a laterally-directed plasma plume 10.

FIGS. 13A, 13D, 13E show examples wherein the discharge electrode(s) 1303 and corresponding dielectric barrier layer 1301 are positioned around (e.g., circumferentially surrounding) one or more apertures 1302 through which ionization gas flows to be ionized and produce one or more plasma plumes 10. In FIG. 13A, coaxial cable interconnects discharge electrode 1303 with a high voltage supply, and gas delivery tube 1307 comprises a single aperture 1302 on a lateral side, just before a closed-off blunt distal end of gas delivery tube 1307. In FIG. 13D, gas delivery tube 1331 comprises a closed-off sharp distal end (optionally hollow or solid) with a lateral-facing aperture. The sharp distal end is optionally used for navigation within a body lumen and/or tissue penetration. In FIG. 13E, gas delivery tube 1341 comprises a plurality of apertures 1302; as shown, two lateral apertures and one distal end aperture. Optionally, the corresponding plurality of discharge electrodes 1303 are interconnected through linking connections to receive the high voltage supply of electrical power.

FIG. 13F shows a lateral aperture configuration, wherein a discharge electrode assembly 601 is positioned within a gas delivery tube 1351, and the resulting plasma plume 10 directed outward from a lateral-facing aperture. A similar electrical design is optionally provided to the aperture and sharpened-end or open-end configurations of FIGS. 13D-13E.

FIGS. 13B and 13C show other aperture designs; an oblique-directing aperture 1312 of a gas delivery tube 1311 (FIG. 13B) and a backward-oblique directing aperture 1322 of a gas delivery tube 1321 (FIG. 13C). Either of the two electrical configurations discussed in relation to FIGS. 13A and 13D-13F is optionally provided to either of the embodiments of FIGS. 13B-13C; that is, gas-around-electrode, or electrode-around-gas.

### Off-Axis Discharging Plasma Delivery Tips

Reference is now made to FIG. 14A, which schematically illustrates scanning delivery of cold plasma to a body lumen 50, according to some embodiments of the present disclosure.

In some embodiments, a plasma plume 10 direction can be altered by steering. FIG. 14A illustrates movement of a distal end of a plasma delivery tip 66 within a body cavity 50 between a first position 1405A and a second position 1405B (the dotted lines reflect that the same device is shown in two different positions separated by time and device movement). The movement is optionally actuated, for example, by a tip steering mechanism as described in relation to FIG. 3A, or another steering mechanism.

Reference is now made to FIG. 14B, which schematically illustrates a plasma delivery tip 66 configured for angulation scanning from within a sheath tube 101, according to some embodiments of the present disclosure.

In some embodiments, a plasma delivery tip 66 is steerable from within a sheath tube 101, allowing it to bend internally to change an angle of a plasma plume 10, optionally without requiring corresponding movement of sheath tube 101. This is a potential advantage, e.g., for directing plasma to different positions from within a confined space and/or stiff access way (e.g., an access way through bone) that restricts movements of sheath 101.

Optionally (e.g., as shown in the difference between the left and right panes of FIG. 14B), actuation comprises changing tension on a member attached near a distal end of plasma delivery tip 66; for example a portion of an electrical conduit 105. The example of FIG. 14B also illustrates an insulating sheath layer 102 defining an electrode-receiving space 107, a dielectric barrier 103, and a discharge electrode 106; other configurations of these and/or other features (for example as described in relation to FIGS. 1A-2H) are optionally provided instead. The angulation scanning is optionally provided by a mechanism that does not use the electrical conduit 105 itself, but is separately provided.

Reference is now made to FIG. 14C, which schematically illustrates a plasma delivery tip 66 configured for wire-guided scanning by bending of a gas delivery tube 603, according to some embodiments of the present disclosure. Optionally, the flexing occurs within a short distal region of the plasma delivery tip, e.g., the distal-most 15 mm, 10 mm, or 5 mm of the plasma delivery tip. Flexing a short portion of the plasma delivery tip has the potential advantage of scanning a plasma plume over a target without requiring much additional room for maneuvering.

In some embodiments, angulation of a distal aperture of a gas supply tube 603 is adjustable by movements actuated from near an exit aperture of gas delivery tube 603. In some embodiments, a control wire 1401 extends from a proximal side slidingly through an anchor position along gas delivery tube 603. The anchor position optionally comprises, for example, an aperture of a first tube insert 1402 (e.g., an inserted ring). Control wire 1401 is fixed to another anchor position further along gas delivery tube 603, which may comprise, for example, a second tube insert 1403 (e.g., an inserted ring). The anchoring positions differ by a radial offset (left panel). Tightening control wire 1401 tends to bring the anchoring positions closer together, resulting (right panel) in a change in the alignments of the anchor positions (e.g., the inserts change their relative radial alignment). This in turn induces a deflection in gas delivery tube 603 (right panel), inducing a deflection in the angle of the plasma plume 10.

FIG. 14C illustrates steering of a plasma plume 10 for embodiments comprising a discharge electrode assembly 601 positioned within a lumen of a gas supply tube 603. The same mechanism is optionally provided within a lumen of an embodiment where the discharge electrode (e.g., a discharge electrode 106) surrounds the flow of gas; for example, replacing the external tensioning cable arrangement shown in FIG. 14B. Conversely, steering of embodiments comprising a discharge electrode assembly 601 positioned within a lumen of a gas supply tube 603 is optionally provided by a cable mechanism, for example as described in relation to FIG. 14B.

Reference is now made to FIGS. 15A-15C, which schematically illustrate a plasma delivery tip 66 configured for rotationally actuated scanning of a plasma plume, according to some embodiments of the present disclosure.

In some embodiments, a position of a plasma plume 10 generated by a plasma delivery tip 66 is scanned from within a sheath tube 101 by a rotational actuation comprising rotation of a guide insert 1501. Plasma delivery tip 66 passes from a proximal direction through a radially offset aperture of guide insert 1501, and guide insert 1501 is in turn positioned within sheath tube 101. Rotation of guide insert 1501, accordingly, induces a circumferential movement of plasma delivery tip 66 within sheath tube 101. Optionally, rotation is by direct rotation of guide insert 1501. Optionally, rotation of a conduit attached to plasma delivery tip 66 (e.g., a portion of probe conduit 73) causes rotation of plasma delivery tip 66 itself, and guide insert 1501 is attached to plasma delivery tip 66 so that it also rotates. FIGS. 15A-15C illustrate this circumferential motion in two steps of about 90° each.

Optionally, circumferential motion at a distal end of plasma delivery tip 66 is converted to a more linear motion by use of an additional slotted guide insert 1503 distal to guide insert 1501. Slotted guide insert 1503 “filters” one axis of the circumferential motion, reducing its amplitude, while motion along the other axis remains free. Optionally, there is produced an angulation along the axis of reduced circumferential motion, due to the radial offset forced in plasma delivery tip 66 between the position of insert 1501 and the position of insert 1503. The amplitude of this angular “wiggle” is optionally adjusted by changing the relative positions of the two inserts along a proximal-to-distal axis, for example as described in relation to FIGS. 6E-6G. Optionally, angulation is induced for both axes (e.g., producing a cone-shaped pattern movements of plasma plume 10) by fitting the aperture of guide insert 1503 to about the size of plasma delivery tip 66 in both directions.

### Adjusting a Plasma Delivery Tip

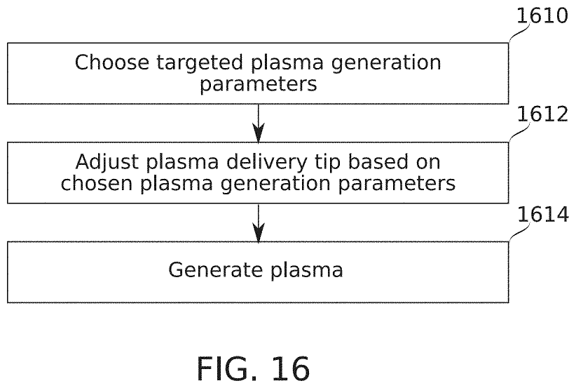

Reference is now made to FIG. 16, which is a schematic flowchart of a method of adjusting a plasma delivery tip, according to some embodiments of the present disclosure.

At block 1610, in some embodiments, targeted plasma generation parameters are chosen. In some embodiments, the choice is direct; e.g., an explicit choice to increase or decrease the thickness of a dielectric barrier layer, to increase or decrease the lumenal diameter of a gas delivery lumen, and/or to increase or decrease the longitudinal extent of a discharge electrode. In some embodiments, the choice is by a proxy parameter; e.g., a choice to increase power and/or decrease temperature. Optionally, the choice comprises selection of a predetermined protocol or range of protocols; for example, a narrow-plume or wide-plume delivery of plasma, or a protocol which has been prepared for the purpose of treating a particular target type and/or organ tissue type.

At block 1612, in some embodiments, the plasma delivery tip is adjusted, based on the chosen plasma delivery parameters. Adjustment is optionally direct, e.g., a slider or other control is manipulated by an operator to adjust a thickness, diameter, or other dimension. In some embodiments, adjustment is performed by making a selection along a scale (e.g., of power, temperature, or an arbitrary scale), and/or a selection from among a plurality of options. In some embodiments, the plasma treatment device automatically manipulates a plurality of parameters based on the selection along the scale or the selected option. Optionally, parameters for other than the plasma delivery tip configuration itself are adjusted; for example, voltage output by a power supply, a delivery rate of ionization gas, and/or a mix of ionization gas species. In some embodiments, adjustment is implemented through use of logic circuitry; e.g., a selection is made through a controller interface, and a controller actuates changes to the plasma delivery tip (and other optional changes) based on the selection. Details of the actuation are optionally stored as computerized (digital) instructions and/or data.

At block 1614, in some embodiments, the plasma is generated, using the adjusted plasma delivery tip.

### Plasma Delivery Tip Cross-Sectional Shapes

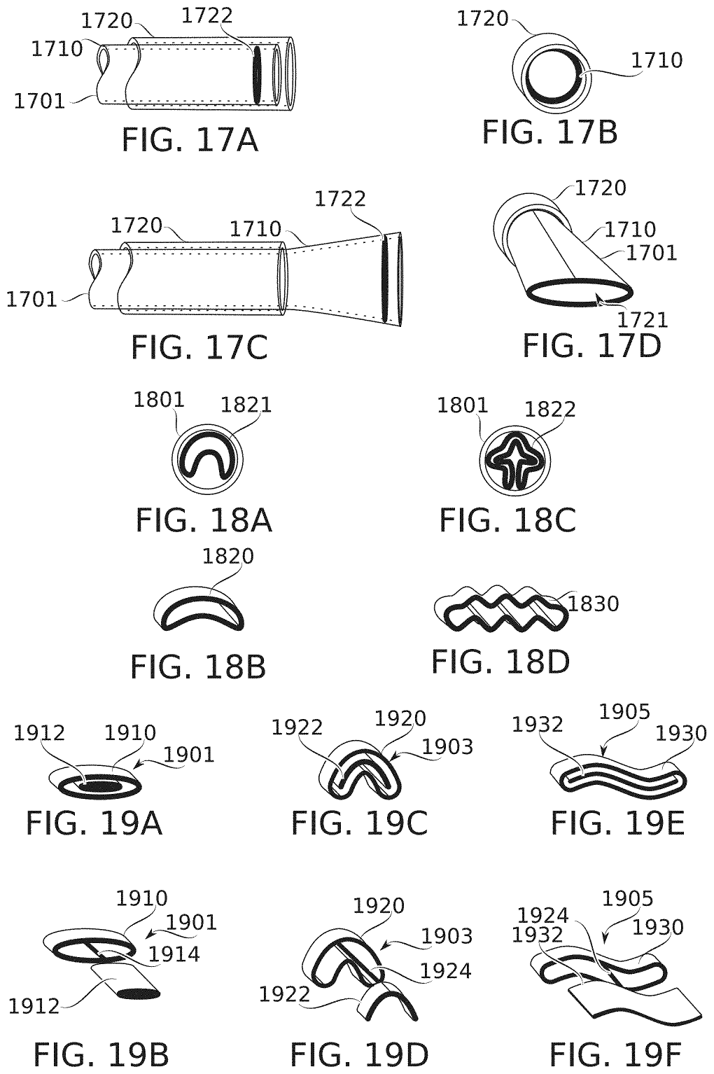

Reference is now made to FIGS. 17A-17D, which show a plasma delivery tip 1710 that deploys its distal aperture 1721 to a cross-section wider than the diameter of the sheath tube 1720 from which tip 1700 deploys, according to some embodiments of the present disclosure. Reference is also made to FIGS. 18A-18D, which show other examples of wide cross-section distal apertures 1820, 1830, according to some embodiments of the present disclosure.

FIGS. 17A-17B show the same configuration of tip 1710 in transverse view (FIG. 17A) and end-on longitudinal view (FIG. 17B). In this configuration, plasma delivery tip 1710 is confined by the (circular) lumenal wall of sheath tube 1720.

FIGS. 17C-17D show another same configuration of tip 1710 in transverse view (FIG. 17C) and end-on longitudinal view (FIG. 17D). In this configuration, plasma delivery tip 1710 is extended from sheath tube 1720, allowing it to assume an oblong (e.g., elliptical) configuration. This change in shape may be driven by elastic energy stored when the material (e.g., silicone polymer rubber, or another flexible polymer) of the lumenal wall 1701 of tip 1710 was compressed to fit within the lumen of sheath tube 1720. In some embodiments, a support ring 1722 is provided to assist expansion and/or stability of the expanded tip. Support ring 1722 may be made, for example, of a superelastic metal such as nitinol. Optionally, support ring 1722 also acts as the discharge electrode of plasma delivery tip 1710. Here and in FIGS. 18A-18D, indications of the electrical conduit 105 and/or alternative discharge electrode 106 embodiment are suppressed for clarity. These elements are optionally provided according to principles described, for example, in relation to FIGS. 1B-3B.

A potential advantage of the expanding tip is broadening of the plasma plume generated therefrom so that it spreads across a larger linear extent than the inner diameter of sheath tube 1720 from which tip 1710 was delivered. This may be a joint function of a change in the distribution of ionization gas, and a change in the shape of the electrode (e.g., attached to the lumenal wall of tip 1710) which ionizes the ionization gas.

Moving this broadened plasma plume along an axis substantially transverse to the direction of broadening in strokes may allow “painting” tissue surface areas with plasma with relatively few strokes, greater overlap, and/or greater certainty of not leaving gaps between adjacent strokes. Another potential advantage is that the average closest distance of approach between ionization gas molecules and discharge electrode itself is potentially decreased (e.g., molecules in the center are not as far from the electrode as would be the case for a circular lumen). In turn, this potentially helps to increase efficiency of ionization effects exerted by the electrode on the ionization gas, and/or efficiency of a cooling effect whereby the ionization gas carries away heat developed due to power losses near the electrode.

FIGS. 18A-18D show other examples of cross-sectional shapes which may be provided, optionally according to the same principles described in relation to FIGS. 17A-17D. FIGS. 18A-18B show a crescent-shaped plasma tip cross-section confined by sheath tube 1801 (resulting in collapsed cross-section 1821 of FIG. 18A), and after release of confinement (expanded cross section 1820 of FIG. 18B; compare to the inner diameter of sheath tube 1801). FIGS. 18C-18D show a zig-zag shaped plasma tip cross-section confined by sheath tube 1801 (resulting in collapsed cross-section 1831 of FIG. 18C), and after release of confinement (expanded cross section 1830 of FIG. 18D; compare to the inner diameter of sheath tube 1801).

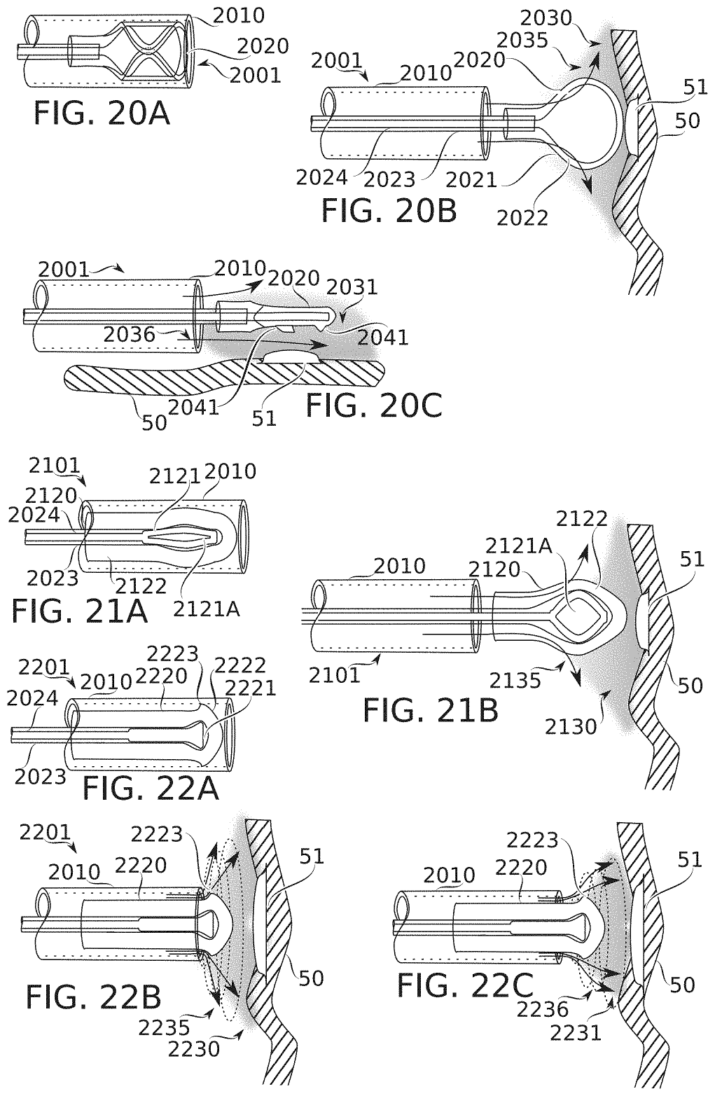

Reference is now made to FIGS. 19A-19F, which show examples of wide cross-section distal apertures of plasma delivery tip tubes 1910, 1920, 1930 which themselves house discharge electrode assemblies 1912, 1922, 1932 having elongated cross-sections, according to some embodiments of the present disclosure. In some embodiments, electrode assemblies 1912, 1922, 1932 comprise an inner conductor connected to electrical conduit 1924 and embedded in an outer insulator (an example of a dielectric barrier layer 103), for example as described in relation to the discharge electrode assemblies 2020, 2120, 2220 of FIGS. 20A-22C. Optionally, the dielectric barrier layer is omitted. The plasma generation parameters are expected to be different for the two type of discharge electrode assemblies, and without the dielectric barrier layer, more current is expected to pass into the body of a patient than when a dielectric barrier layer is used.

In some embodiments, any of discharge electrode assemblies 1912, 1922, 1932 is movable separately from the respective plasma tip lumen that houses it. For example, discharge electrode assemblies are optionally actuatable via movements of their respective connected electrical conduits 1924 to advance out of their respective sheaths; e.g., as described in relation to FIGS. 11A-11D.

Electrode assemblies may be flexible to collapse along with their enclosing tip lumen (e.g., as in the case of plasma delivery tips 1903, 1905 and their discharge electrode assemblies 1922, 1932), or may be small enough in width that they do not need to collapse in order to be held within a confining sheath (e.g., as in the case of plasma delivery tip 1901 and its discharge electrode assembly 1912).

The embodiments of FIGS. 19A-19F partially decouple the shape of the discharge electrode from the shape of the lumen through which ionization gas flows, since the electrode is not embedded in the wall itself. It should be noted in particular that plasma can still be generated when discharge electrode assembly is extended out of its lumen, resulting in a plasma plume with a shape that is in part determined by the shape of the electrode, and in part by the way that the lumen of the plasma delivery tip shapes the flow of ionization gas. The decoupling potentially allows shaping the region of plasma generation and/or delivery to suit particular needs, such as particular tissue surface shapes. Furthermore, ionized species generated in plasma potentially change along the length of a plasma plume as it undergoes post-ionization equilibration. For example, along just a few millimeters of plasma plume, there may occur quenching of primary ionization species, generation of secondary ionization species as primary-generated ions interact with each other and/or initially non-ionized species, and/or redistribution of thermal energy. Any of these has a potential to influence therapeutic effects, according to circumstance. Advancing the discharge elect out of the gas delivery tube potentially allows control of how “fresh” plasma is when it reaches tissue, since it is the discharge electrode itself which is the site of initial plasma generation.

### Discharge Electrodes

Reference is now made to FIGS. 20A-20C, which schematically illustrate a width-expanding discharge electrode assembly 2020 for use with a plasma delivery tip 2001, according to some embodiments of the present disclosure. Reference is also made to FIGS. 21A-21B, which schematically illustrate a different width-expanding discharge electrode assembly 2120 for use with a plasma delivery tip 2101, according to some embodiments of the present disclosure.

Width-expanding discharge electrode 2020 comprises a flexible conducting sheet 2022 (optionally comprising a superelastic alloy such as nitinol), optionally insulated by a dielectric barrier layer 2021. When confined within a lumen of tube 2010, width-expanding discharge electrode assembly 2020 folds and/or rolls up, for example as illustrated in FIG. 20A.

Upon leaving the confinement of tube 2010, discharge electrode assembly 2020 flattens out. FIGS. 20B-20C show use of the flattened discharge electrode assembly 2020 to generate plasma plumes.

In FIG. 20B, discharge electrode assembly 2020 can approach quite closely to a surface of tissue wall 50 (in the example shown, a surface including a treatment target 51) while a flow of ionization gas 2035 (2035 refers to any of the arrows indicating flow) emitted from tube 2010 passes over it. The flow of ionization gas 2035 may be somewhat dispersed by deflection from tissue wall 50, and/or as a function of distance from the aperture of tube 2010 from which it emits. Since all of conducting sheet 2022 is at about the same electrical potential, and insofar as its surface is well-covered by flowing ionization gas, the linear extent of plasma plume 2030 (in transverse cross-section) is potentially increased, compared to the more “pencil like” flow which may emit directly from the distal aperture of tube 2010. Moreover, at least a portion of the plasma may be generated in very close proximity to tissue wall 50, optionally up to and including being generated at locations essentially in contact with treatment target 51.

In FIG. 20C, the flat surface of discharge electrode assembly 2020 is oriented approximately parallel to the surface of tissue wall 50. This has the potential to create a plasma plume 2031 having a relatively large surface area in contact with tissue wall 50, and in particular the region of treatment target 51. This is a potential advantage for selective delivery of plasma together with confidence that the whole surface area of a treatment target 51 surface area has been sufficiently saturated with treatment plasma. It may be noted that discharge electrode assembly 2020 and tissue wall 50 act together to partially confine gas which flows between them, which potentially helps to increase the concentration of plasma.

Ionization gas distribution is optionally assisted by one or more baffles positioned on any suitable surface of discharge electrode assembly 2020, for example, baffles 2041 (baffles 2041 not shown in FIG. 20B, but optionally they are provided there as well). Baffles in the form of protrusions (e.g., fins) from the assembly which are formed to help distribute a stream of ionization gas to form a broader plume and/or spread ionization gas over a larger extent of the electrode surface are optionally provided as a component of any of the discharge electrode assemblies described herein. In particular baffles may be useful with discharge electrode assemblies which expand and/or re-orient upon their advance out of a confining tube. The baffles can help guide a stream of ionizing gas to flow over more portions of the electrode's useable surface area.

In some embodiments, discharge electrode assembly 2020 tapers on a proximal side, so that interference with the walls of tube 2010 forces it to re-roll and/or re-fold as it is withdrawn back into confinement.

Conductor 2024 is used to deliver discharge voltage to the discharge electrode assembly 2020, as a part of an electrical conduit 2023 (which is in turn an embodiment of an electrical conduit 105). Electrical conduit 2023 is optionally a coaxial cable, or an insulated single-conductor cable.

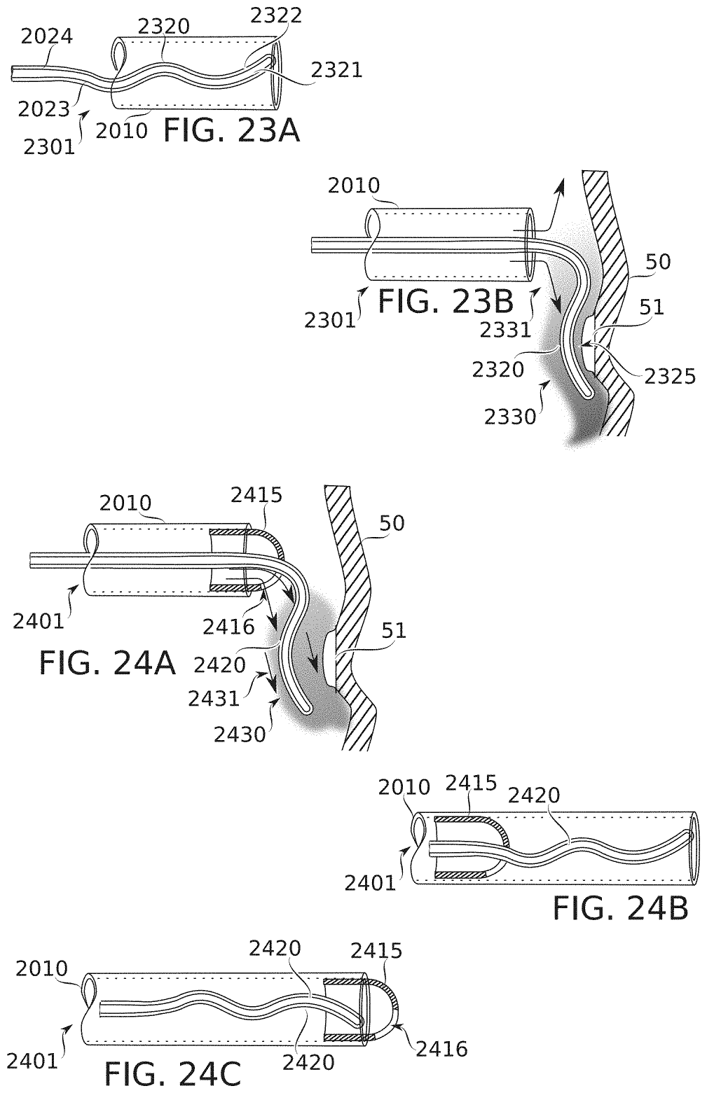

It should be noted that there is no limitation to orient the “plate” surfaces of discharge electrode assembly 2020 along the longitudinal axis of the lumen from which it advances. Discharge electrode assembly 2020, for example, may be preconfigured to bend toward the perpendicular (e.g., assume an angle such as is illustrated for the linear ablation elements of FIGS. 23A-25), so that it can be directly advanced up to a surface with a flat side facing it. This would be similar to the situation shown in FIG. 20, but with tube 2020 oriented more closely to the perpendicular relative to the surface of tissue 50. The angle of bending may be any selected angle, e.g., between 0° and 90°. Optionally the angle of bending is acute (bending greater than) 90°, which is a potential advantage to allow “reaching behind”—for example to allow reaching tissue which is very near to the body lumen aperture from which the plasma delivery tip is introduced, and/or to allow a greater range of steerability. Reorientation upon advance from a lumen is optionally applied to other embodiments where an electrode assembly can be advanced out of a lumen which delivers it to a treatment site, e.g., any of FIG. 19A-19F or 21A-21B.

FIGS. 21A-21B show another expandable electrode: expanding discharge electrode assembly 2120. In some embodiments, a conducting portion 2121 of the expanding discharge electrode assembly 2120 comprises a loop of superelastic alloy which is elastically predisposed to expand, opening its loop aperture 2121A. Conducting portion 2121 is optionally embedded within an envelope 2122 of insulating material to form a dielectric barrier.

When confined by tube 2010 (which may also be an ionization gas delivery tube), the loop of conducting portion 2121 is held closed. Upon exiting the confinement of tube 2010, the loop opens, with the effect of broadening (and optionally also flattening) the surface area of discharge electrode assembly 2120 over which ionization gas can be converted to plasma. Withdrawal back into tube 2010 squeezes the loop of conduction portion 2121 closed again.

It should be noted that the expanding shapes of the discharge electrode assemblies of FIGS. 19A-19F undergo “assisted collapse”, helped along by the collapse of the expanded cross-sectional shapes of the tubes which enclose them. The discharge electrode assemblies of these figures can be freely advanced from and/or retracted into their corresponding tubes when those tubes are themselves deployed to their expanded shapes, because the two shapes are sufficiently matching to allow it. Once retracted into their housing tube, a discharge electrode assembly that is wider than the sheath that delivered it can be collapsed to its allow complete retraction (and, e.g., withdrawal from the body) by the exertion of collapsing force from the tube that surrounds its—as that tube itself is collapsed by withdrawal into the sheath from which it emerges. In contrast, the embodiments of FIGS. 20A-21B comprise discharge electrode assemblies which, although non-circular themselves, can advance out of and retract into circular lumens having a diameter smaller than their own width, when fully expanded. These discharge electrode assemblies may be particularly appropriate for use with circular working channels of other devices; e.g., standard working channels of a colonoscope.

Reference is now made to FIGS. 22A-22C, which schematically illustrate a flow-spreading electrode assembly 2220 for use with a plasma delivery tip 2201, according to some embodiments of the present disclosure.

In some embodiments, an envelope 2022 of a discharge electrode assembly 2220 is provided with a flow-spreading flange 2223 which substantially fills the lumen of ionization gas delivery tube 2010 when discharge electrode assembly 2220 is confined therein. Upon its initial advance out of ionization gas delivery tube 2010, flow-spreading flange 2223 creates a relatively narrow aperture which redirects the flow of ionization gas (indicated by arrows 2235) into a spread-out configuration. Plasma 2230 generated in the flow of ionization gas by discharge electrode 2221 is thereby distributed as well. With greater advance of flow-spreading flange 2223, the degree of spreading of ionization gas flow 2236 decreases, resulting in a finer plasma plume 2231. By inter-converting between these configurations (and optionally greater advance of flow-spreading flange 2223), a circular area can be swept with plasma. If present, a “shadowed” region directly in front of the discharge electrode assembly 2220 can be given plasma coverage by slight lateral offsets of gas delivery tube 2010. Such movements may occur naturally, e.g., due to normal body movements, as the plasma treatment is delivered, and/or be deliberately generated by the device operator.

It should be noted that the gap between flange 2223 and the lumenal wall of tube 2010 may be circular, or it may be interrupted, e.g., by making flange 2223 extend further distally in some places. Interrupting the gap may allow the flow of ionization gas to be preferentially directed, e.g., in two opposite directions, giving the plasma plume cross-section a longer axis and a shorter axis. This shape may be suitable for making sweeps across a target. Concentration of the flow of ionization gas through smaller apertures may concentrate and/or extend the plasma plume to reach a wider extent along the longer axis compared to a circular aperture for a similar total amount of gas flow.

Reference is now made to FIGS. 23A-23B, which schematically illustrate an off-axis deploying discharge electrode assembly 2320 for use with a plasma delivery tip 2301, according to some embodiments of the present disclosure.

In some embodiments, discharge electrode assembly 2320 comprises a superelastic electrical conductor (e.g., comprising nitinol) 2321, optionally within an envelope 2322 of an insulating material (e.g., a polymer coating) which acts as a dielectric barrier. While confined within ionizing gas delivery tube 2010, discharge electrode assembly 2320 is held relatively straight. Upon emerging from a distal end of ionizing gas delivery tube 2010, discharge electrode assembly 2320 assumes a bend that angles it away from a central longitudinal axis of ionizing gas delivery tube 2010, e.g., to make an angle of at least 45° with the central longitudinal axis, optionally up to about 70°, 80° or 90°. In some embodiments, discharge electrode assembly 2320 protrudes far enough from gas delivery tube 2010 that it extends radially beyond the lumenal cross section of gas delivery tube 2010; e.g., extends radially away from the central axis by more than a radius of the lumenal cross-section.

In some embodiments, the discharge electrode assembly 2320 bends more than 90°, e.g., all the way back (proximally) behind the aperture from which it emerges. This is potentially advantageous for reaching back behind to regions adjacent to an aperture used to enter a body lumen. To bring ionization gas to the same region, the whole lumen may be filled with ionization gas, or ionization gas may be diverted out of a lateral aperture, optionally a lateral aperture provided with a partially backward-facing (proximally-facing) angulation.

One option to keep the discharge electrode in the flow of gas is to bring the ionization gas exit aperture of tube 2010 close enough to the surface of tissue 50 comprising treatment target 51 that the flow (e.g., as indicated by arrows 2331 in FIG. 23B) is laterally deflected. Within a sufficiently enclosed lumenal space, redirection of flow is not necessarily required, once ionization gas has been diffused sufficiently therein. Portions of the flow of ionization gas which pass along discharge electrode assembly 2320 can then be ionized when appropriate voltage is delivered to discharge electrode assembly 2320 from electrical conduit 2323 along conductor 2324. As a result, a linearly extended region of contact may be generated between plasma plume 2330 and adjacent tissue. Linear scanning movements of tube 2010 transverse to the axial extent of Discharge electrode assembly 2320 can be used to produce coverage over a still wider surface area.

Optionally, discharge electrode assembly 2320 is shaped with a curvature to match its target. For example, an inwardly protruding treatment target 51 (e.g., a raised region of cancerous tissue) can be accommodated by shaping discharge electrode assembly 2320 with a concavity 2325. Additionally or alternatively, straight and/or convex shapes may be provided.

Reference is now made to FIGS. 24A-24C, which schematically illustrate an off-axis deploying electrode assembly 2420 for use with a plasma delivery tip 2401 having an off-axis directed ionization gas exit aperture 2416, according to some embodiments of the present disclosure.

In some embodiments, the flow of ionization gas 2431 is redirected off-axis to match the off-axis positioning of a discharge electrode assembly 2420. In the example shown, a cap 2415 is provided with an off-axis aperture 2416 on its side. The side placement of aperture 2416 helps to redirect the flow of ionization gas along discharge electrode assembly 2420 (which optionally also exits tube 2010 via aperture 2416.

Cap 2415 may be fixed in place at a distal end of tube 2010 (FIG. 24C). Alternatively, it may be attached to a more proximal portion of discharge electrode assembly 2420 (FIG. 24C), so that the two advance distally together. A potential advantage of the configuration of FIG. 24C is suitability for use with a multi-purpose working channel. Attachment of cap 2415 to discharge electrode assembly 2420 allows it to be completely withdrawn from the working channel during a procedure, freeing the working channel for other uses that a procedure may call for. There is also a potential advantage to avoid having to thread discharge electrode assembly 2420 through the narrowed aperture 2416 provided by cap 2416, although this can be mitigated, e.g., by providing tapered internal surfaces as guides. The fixed-cap configuration of FIG. 24C may be simpler to arrange in cases where working channel reconfiguration is not needed.

In some embodiments, discharge electrode assembly 2420 is free to rotate relative to aperture 2416, at least as far as the limits of the size of the aperture allow. Optionally, this is used as a method of power adjustment. When discharge electrode assembly 2420 is maximally within the stream of ionization gas leaving aperture 2416, the ionizing zone is longest. There may be corresponding maximal generation of plasma—but potentially also greater heating. In some embodiments, rotating discharge electrode assembly 2420 partially out of the flow of gas reduces the amount of dissipated power. Optionally, this is used as a method of power regulation (and correspondingly, temperature regulation). It is noted that the electrically controlled parameters (e.g., voltage) governing power delivery may not be as readily adjusted, due to non-linearities in the mechanisms governing plasma generation. For example, below a certain threshold voltage, plasma generation may abruptly cease.

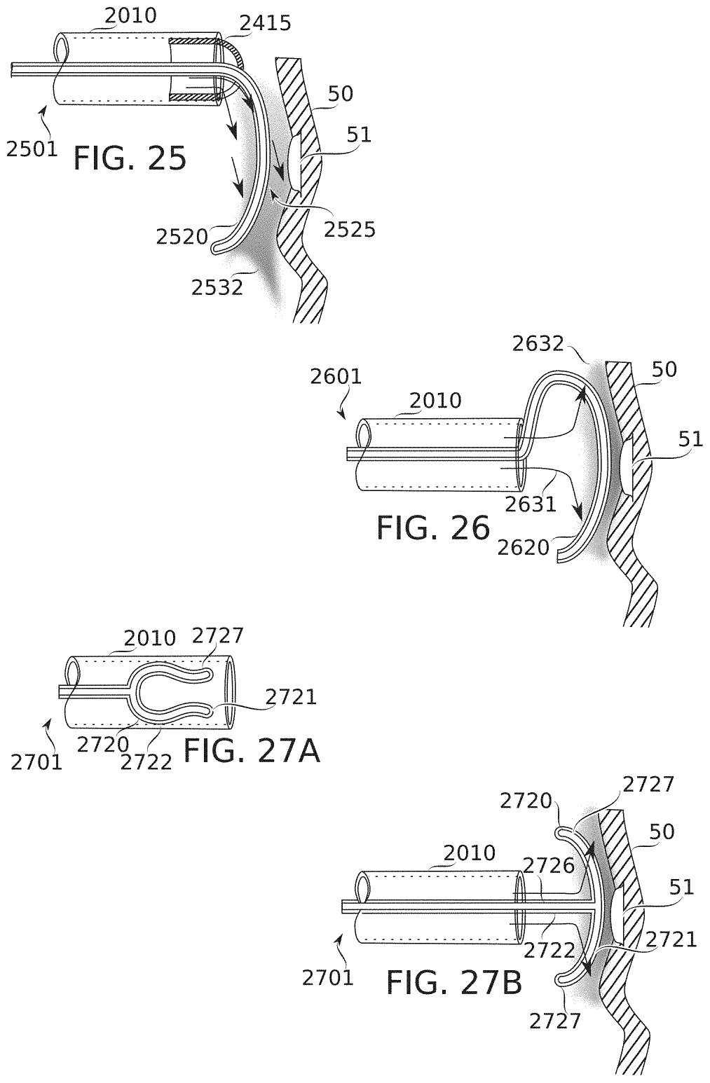

Brief reference is now made to FIG. 25, which schematically illustrates an off-axis deploying discharge electrode assembly 2520 for use with a plasma delivery tip 2501, according to some embodiments of the present disclosure. Compared, e.g., to the discharge electrode assembly shown in FIGS. 23A-24C, discharge electrode assembly 2520 comprises a convexity 2525, which may be more suitable for introduction of plasma plume 2532 into a tissue concavity, e.g., a concavity which may otherwise act to partially shelter a treatment target 51. It should be understood that discharge electrode assembly 2520 may be introduced while confined within tube 2010, extended for plasma treatment to the configuration shown, and then withdrawn again into tube 2010 in preparation for withdrawal from the treatment area. In some embodiments, discharge electrode assembly 2520 is rotatable around a longitudinal axis of tube 2010, optionally together with cap 2415. This may allow, e.g., areal coverage while tube 2010 remains in a single location.

Brief reference is now made to FIG. 26, which schematically illustrates a self-expanding discharge electrode assembly 2620 for use with a plasma delivery tip 2601, according to some embodiments of the present disclosure.

In the example shown, re-curvature of discharge electrode assembly 2620 (that is, a first curve to a radially outward direction, followed by a second curve to a radially inward direction) allows providing a lengthened longitudinal extent of plasma-generating electrode which remains approximately centered on a longitudinal proximal-to-distal axis of tube 2010. Centering discharge electrode assembly 2620 potentially increases the amount of ionizing gas flow (arrows 2631) which is available for generating plasma plume 2632. The curvature of discharge electrode assembly 2620 along which plasma is generated may be convex against tissue as shown, and/or concave, for example as described in relation to FIGS. 23A-24C. Discharge electrode assembly 2620 is alternately extendable from and withdrawable into tube 2020, for example as described in relation to FIG. 25 and other figures herein.

In some embodiments, discharge electrode assembly 2620 is rotatable around a longitudinal axis of tube 2010. This may allow, e.g., areal coverage while tube 2010 remains in a single location.

Optionally, discharge electrode assembly 2620, is deployed from a lumen which is not itself a gas delivery tube. Optionally, the gas delivery tube is provided separately from or alongside discharge electrode assembly 2620. In some embodiments, a distal end of discharge electrode assembly 2620 is formed as a loop, and gas delivery tube is provided circumferentially within the loop.

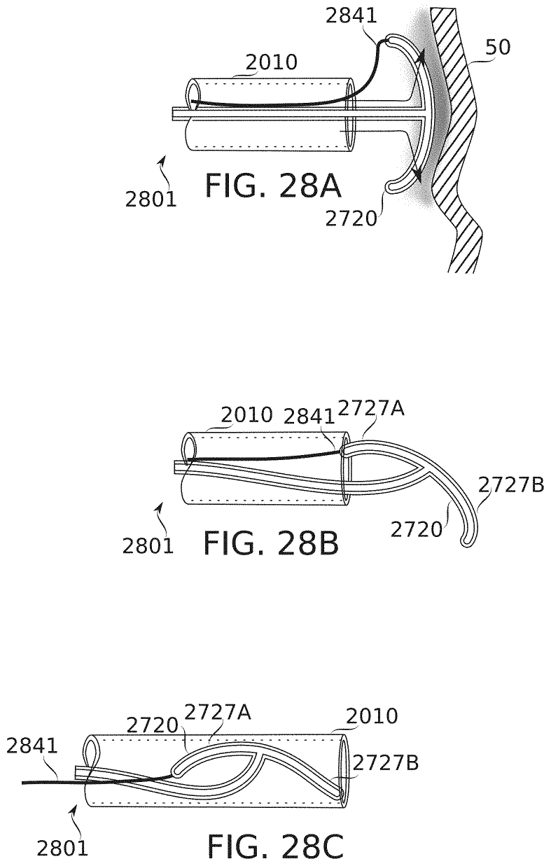

Reference is now made to FIGS. 27A-27B, which schematically illustrate a self-expanding discharge electrode assembly 2720 for use with a plasma delivery tip 2701, according to some embodiments of the present disclosure. Reference is also made to FIGS. 28A-28C, which schematically illustrate a plasma delivery tip 2801 that packages the self-expanding discharge electrode assembly 2720 differently than shown in FIGS. 27A-27B, according to some embodiments of the present disclosure.

In these examples, discharge electrode assembly 2720 comprises a plurality of flexible members 2727, each extending away from a central member 2726 to which the flexible members 2727 are connected. Flexibility and elasticity is conferred, for example, by using a superelastic alloy to form the conductive element 2721 of the discharge electrode assembly 2720. Optionally, an insulating envelope 2722 is provided as a dielectric barrier.

Collapsed and confined to tube 2010 as shown in FIG. 27A, flexible members 2727 may be deflected distally to allow them to fit within the lumen of tube 2010. Upon being freed from confinement, flexible members 2727 self-deploy by bending proximally and radially outward from a central longitudinal axis of a distal portion of tube 2010 to assume their deployed configuration. In the example shown, these members deploy to present a convex surface oriented toward treatment target 51. It may be understood that a straight or concave surface is additionally or alternatively presented toward the treatment target 51, for example as described in relation to FIGS. 23A-24C.

Alternatively to the packaging shown in FIG. 27A, members 2727 (further distinguished as members 2727A and 2727B in FIGS. 28A-28C) may be confined so that at least one of them lies along the shaft of central member 2726. In FIGS. 28A-28C, a control member 2841 is provided which can be used to assist in the re-collapse of discharge electrode assembly 2720 to allow its withdrawal. Putting tension on control member 2841 draws member 2727A toward the distal aperture of tube 2010 (FIG. 28B), enabling withdrawal of discharge electrode assembly 2720 into tube 2010 (FIG. 28C), including trailing member 2727B which may deflect in order to enter the lumen of tube 2010. Optionally, control member 2841 is also used as an electrical conduit for conveying discharge voltage to the discharge electrode assembly 2720

In some embodiments, discharge electrode assembly 2720 is rotatable around a longitudinal axis of tube 2010. This may allow, e.g., areal coverage while tube 2010 remains in a single location.

### Adjustable Direction Plasma Tips

Reference is now made to FIGS. 20A-20C, which schematically illustrate a

Introductory reference is now made to FIGS. 29A-50, with respect to some of the particular combinations which may be made among features described with respect to different examples herein.

For brevity and clarity of description, many of the embodiment examples in FIGS. 29A-50 are represented generically (e.g., without particular indication of electrical supply conduit(s) and/or discharge electrodes) except for the particular feature or features which the examples are provided to illustrate.

For example, in any of these figures, gas supply tubes labeled with reference characters in the range 119A-119Z should also be understood as examples of a gas supply tube more generically.

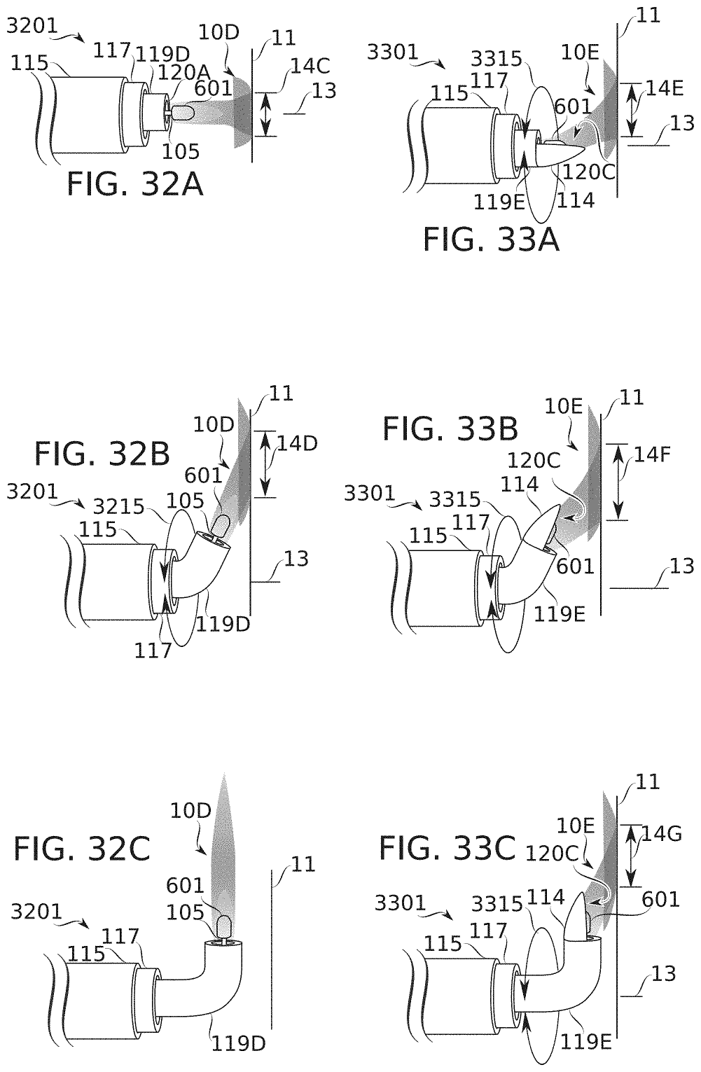

Some of these gas supply tubes (e.g., described in relation to FIGS. 32A-39, 46A-46B, 48 and/or 50) are of the type of gas supply tube 603 which is provided together with a separate discharge electrode assembly 601 (when such an assembly is shown). For embodiments of this type, designs (e.g., of the discharge electrode assembly 601 itself) and principles described, for example, in relation to FIGS. 6A-12B, 13F, 14C, 19A-19F, and/or 20A-28C are optionally combined with the additional features described in relation to the particular gas supply tube, its associated discharge electrode assembly or assemblies, and/or the associated example as a whole.

Other embodiments (e.g., described in relation to FIGS. 29A-29B, 31A-31C, 40A-43, 45A-45B, 47A-47C and/or 49) are of the type where the discharge electrode is circumferentially positioned around a lumenal space defined by the gas supply tube and/or its outlet aperture(s). For embodiments of this type, designs and principles described, for example, in relation to FIGS. 1B-5D, 13A-13E, 14B, 15A-15C, and/or 17A-18D are optionally combined with the additional details described in relation to the particular gas supply tube and/or the associated example as a whole.

Several of the drawings of FIGS. 29A-50 include indication of a working channel 115, shown as a simple lumen, e.g., as may be provided by a catheter. However, it should be understood that working channel 115, in any of these examples, may be a channel of any device with an extended lumen suitable for insertion of a gas supply tube therethrough, for example, an endoscope (e.g., a gastroscope, arthroscope, and/or colonoscope). Thus, for example, working channel 115 may be one channel of a plurality of channels configured together within a single probe body, e.g., of a colonoscope. The working channel 115 may be provided separately from a plasma delivery device which is passed through the working channel 115. Working channel 1103 of FIGS. 11A and 11C may be understood as an example of a working channel 115.

Furthermore, plasma delivery tip examples described in relation to FIGS. 29A-50 do not in general require use of a working channel 115. They can be introduced to their site of operation alone, or in another way, e.g., by being positioned there using forceps.

Several of the drawings of FIGS. 29A-50 include indication of a sleeve 117. It may be understood that other sleeves described herein (e.g., sleeve 101, sleeve 1720, and sleeve 1801) are examples which may be provided to implement a sleeve 117.

The remarks just made in relation to FIGS. 29A-50 should not be understood as excluding by omission any combination not mentioned. For example, the principles described, in some embodiments, extend to combinations of features described in relation to figures herein which may not include any of FIGS. 29A-50.

Reference is now made to FIGS. 29A-29B, which schematically illustrate plasma delivery tips delivered through a working channel 115 within a sleeve 117, according to some embodiments of the present disclosure.

Gas supply tubes 119A, 119B are each examples of a gas supply tube. For clarity, they are represented generically (e.g., without particular indication of electrical supply conduit(s) and/or discharge electrodes) except for the positioning of their respective gas (and/or plasma) outlet apertures 120A, 120B. In some embodiments, plasma delivery tips in the region of outlet aperture 120A (a distally-facing aperture) is implemented according to any of the configurations of, e.g., FIGS. 1B-5D. In some embodiments, lateral side-facing outlet apertures 120B are optionally implemented, for example, as described in relation to FIGS. 13A-13E. Plasma plumes 10, 10A, 10B are also indicated.

A main difference between a sleeve 117 and a working channel 115 is that the working channel 115 can be separately provided, while the sleeve 117 is considered part of the plasma delivery device.

Apart from this, when both are provided, there is optionally a division of functionality between them. The working channel 115 assists bringing tools (in this case, the plasma delivery tip) into position. It is generally provided on a device which offers stiffness and maneuverability suitable for reaching, e.g., a treatment site. The working channel lumen then becomes a relatively low-resistance pathway along which other devices, such as a plasma delivery tip can also be brought to the treatment site.

Sleeve 117 provides protection for the plasma delivery tip itself, e.g., mechanically isolating plasma delivery tip as it passes along working channel 115. This potentially enhances pushability, particularly in the case of embodiments which divide the plasma delivery tip into a plurality of smaller and potentially more delicate channels (e.g., as in FIGS. 36A-50), and/or in the case of embodiments which incorporate elements that are elastically predisposed to self-angulate (e.g., FIGS. 31A-33C), which could resist advancing through a working channel if not encapsulated.

In some embodiments, sleeve 117 comprises a dielectric material (e.g., a polymer), which additionally acts to increase electrical isolation and/or reduce outer-surface inductance of voltage supplied to a plasma delivery tip from the environment. This may prevent accidental plasma discharge in the case of ionization gas leaking back along the working channel. Moreover, it may itself act to help prevent such leakage by filling a portion of the working channel volume which the gas delivery tube itself does not fill. Alternatively, ionization gas being exhausted through a conduit is mixed with gas having a higher breakdown voltage (e.g., by mixture in the plasma-generating environment, and/or by supply directly to the exhaust conduit itself). This potentially also helps prevent ectopic plasma generation along the gas evacuation conduit.

Sleeve 117 may also help to center the plasma delivery tip. Sleeve 117 may be used to support and/or protect electrical connections to a plasma delivery tip used in power delivery and/or sensing (for example, sheath 101 of FIG. 3A encases most of the length of electrical conduits 105). While the separation of these functionalities into separate tubes has some potential advantages (e.g., to allow a same working channel to be used for one or more purposes during a procedure apart from plasma delivery), it should be understood that the functions described separately for working channel 115 and sleeve 117 can optionally be combined into a single tube, particularly for embodiments where working channel 115 does not need to be cleared for use with other tools during a procedure.

FIGS. 29A-29B re-introduce aspects of plasma delivery related to:
providing a plurality of plasma generation sites (also described, e.g., in relation to FIG. 13E),
directing added plasma plumes to locations that suitably complement each other (also described, e.g., in relation to FIGS. 13A-13F, and/or 14A-14C),
“scanning” plasma plumes to cover a larger area than the cross-section of the plume itself provides (also described, e.g., in relation to FIGS. 3A, and/or 14A-14C).

These features indicate ways to address the problem of matching plume size to target size. The problem arises in part as a consequence of miniaturization which reduces a plasma delivery tip's size to a 2-20 mm diameter (typically) suitable for intralumenal use within a body cavity.

The problem also arises in connection with the practical matter that for any particular geometrical configuration of a plasma delivery tip, plasma may actually only be validated for use within a relatively narrow range of plasma generating parameters, and in particular, a range which includes a relatively narrow range of plasma plume diameters. Plasma generation may not occur dependably (or at all) outside of this range; or it may occur but with unknown or insufficient generation of potentially therapeutic plasma species. In general, simply scaling up a small plasma generating tip to a larger plasma generating tip introduces such a large change in plasma generating characteristics that it effectively must be re-validated as a new design.

Thus, for example, from where they exit a round-apertured delivery tube, plasma plumes made from jetting gas tend to adopt a pencil-like shape; for example, the shape of plasma plume 10 of FIG. 29A. This shape is a consequence of many factors, for example: parameters of ionization gas, parameters of ionization gas flow, electrical parameters that generate the initial ionized species in the plasma, geometrical parameters such as electrode and plasma outlet aperture shape, and gas flow and/or electrical interactions of the plasma plume with its environment. The plume shape shown is an “unconfined” shape, e.g., the shape a plasma plume may assume in an open-air environment. However, the shape also changes according to how the flow of gas and/or current is affected by the proximity of other surfaces (e.g., a treated surface). Optionally this also is considered as a significant parameter when validating a plasma plume for use in the delivery of ionized species to a target such as an abnormal tissue area.

Once plasma plume is validated for use (for example as shown in FIG. 29A, and/or as described in relation to other figures herein, e.g., FIGS. 17A-28C), it may be preferable, in some embodiments, to restrict operation to use the parameters that generate it (optionally allowing for parameter adjustments within a range of accepted values), even though the plasma plume's geometry is potentially not optimal for every target—for example, pencil shaped as shown in FIG. 29A.

Adding plasma plumes, controlling the direction of those plasma plumes, and/or scanning those plasma plumes by moving them over a target area are all ways to potentially overcome limitations this practical consideration imposes. The same selected plasma generating parameters can be duplicated at a plurality of outlet apertures, to each similarly produce a plasma plume, and then all these plasma plumes combined to deliver plasma to a target area. Additionally or alternatively, plasma plumes can be moved over a target area in a way that helps to ensure that it receives sufficient coverage.

For example: to a distally-directed plasma plume 10B, the example of FIG. 29B also adds laterally directed plumes 10A. In the example shown, these plumes are directed substantially orthogonally to plasma plume 10B. This configuration can be used to increase the area near the plasma delivery tip that plasma reaches, since (1) there are more plasma plumes, (2) the plasma plumes cover more directions, and (3) gas delivery tube 119A is optionally rotated (double arrow 2904) so that the laterally direct plasma plumes 120A sweep through an approximately cylindrical region.

Sleeve 117 is optionally a tube that extends all the way back to the proximal end of the device (e.g., to handle 80 and its controls). Alternatively, the tubular portion of sleeve 117 may be confined to just a distal region comprising structures of the plasma delivery tip (a “partial length” embodiment of sleeve 117). These structure are optionally unlived when needed by retaining sleeve 117, for example, using a control member extending proximally to handle 80. Optionally, the working channel itself is fitted with a retaining lug that slightly narrows the working channel aperture (e.g., gradually along a taper), to a diameter that still allows structures of the plasma delivery tip to be advanced out of it, while keeping sleeve 117 itself held in place. A potential advantage of partial-length embodiments of sleeve 117 is that the gas delivery tube itself can then be widened, which may decrease resistance to the flow of ionization gas, and/or provide more room for electrical connections.

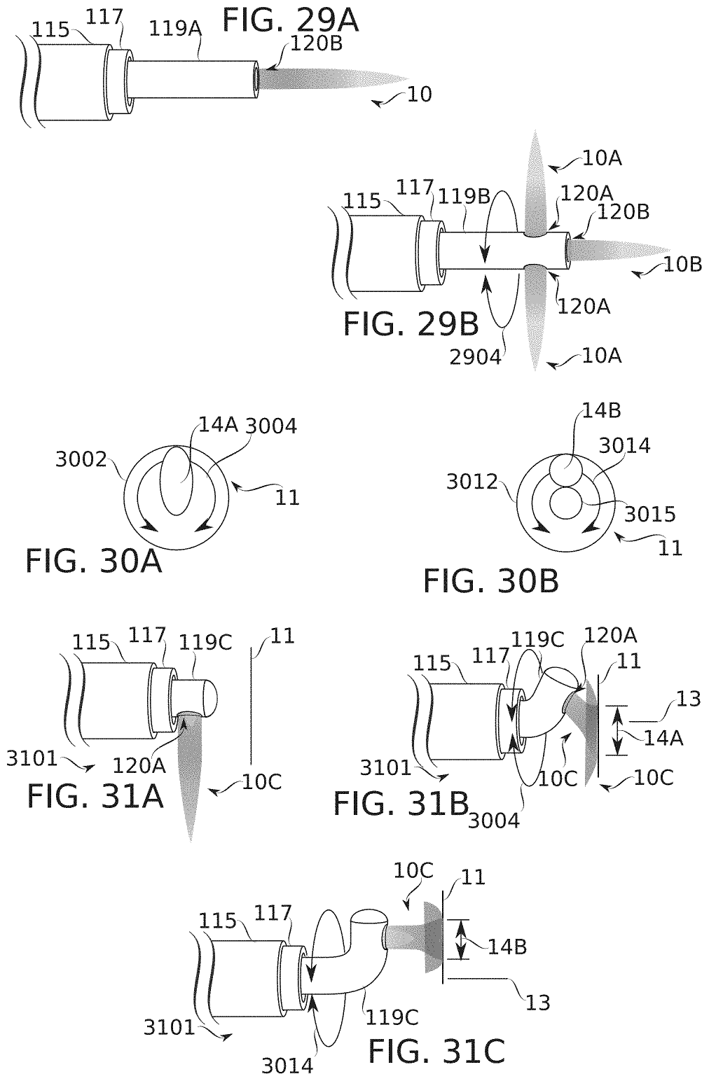

Reference is now made to FIGS. 30A-30B, which schematically represent patterns of plasma interaction with a surface generated by rotation of a plasma plume around a longitudinal axis offset from and/or oblique a longitudinal axis of the plasma plume itself, according to some embodiments of the present disclosure. Reference is also made to FIGS. 31A-31C, which schematically represent a self-orienting plasma delivery tip 3101 which is actuatable to reorient a plasma outlet aperture 120A through a range of off-axis orientations with respect to a longitudinal axis 13 of the sleeve 117 and/or working channel 115 that delivers it, according to some embodiments of the present disclosure.

In the example of FIGS. 31A-31C, gas supply tube 119c is configured with a closed off distal end, and a side-facing outlet aperture 120A near that distal end. Moreover, a section of the distal end of gas supply tube 119C is preconfigured to assume an angled shape when unconfined (e.g., as shown in FIG. 31C), while being flexible enough to straighten out as it is withdrawn into sleeve 117. For example, gas supply tube 119C may comprise a superelastic alloy such as nitinol, allowing it to interconvert between the straightened and angled shapes.

In the partially deployed condition of FIG. 31A, plasma plume 10C is directed approximately laterally from a longitudinal axis defined by the distal ends of sleeve 117 and/or working channel 115. This may be useful to impinge on surfaces substantially perpendicular to surface 11, but in the example shown, surface 11 is considered to be the target—e.g., it may be a tissue surface with a region of abnormal tissue.

Further extending gas supply tube 119C from sleeve 117 allows gas supply tube 119C to assume a partial bend. Now outlet aperture 120A is oriented obliquely to surface 11, so that plasma plume 10C can impinge upon it. In this example, the area of impingement extends within region 14A, which also happens to include the place where imaginary longitudinal axis 13 (a central longitudinal axis of sleeve 117) intersects surface 11.

Looking at surface 11 from a perpendicular angle (FIG. 30A) region 14A resolves as an approximately elliptical area. Gas delivery tube 119C is also rotatable around axis 13 (as indicated by double-headed arrow 3004). As it rotates, plasma plume 10C sweeps out an approximately circular area 3002.

In FIG. 31C, gas delivery tube 119C is extended further, so that it finishes assuming its predefined angle—in this case, an angle of about 90° to axis 13. The final predefined angle can be any suitable angle, e.g., an angle between about 45° and 90°; or more than 90°, e.g., up to about 135°, and/or less than 45°, e.g., about 30°. In this figure, plasma plume 10C impinges on surface 11 also at about a 90° angle. This results in an approximately circular shaped area 14B of plasma impingement on surface 11, as shown from a surface-perpendicular angle in FIG. 30B. Since circle 14B doesn't include the intersection point of axis 13 with surface 11, the shape of rotation it generates when rotated in the directions indicated by double-headed arrow 3014 has a hole 3015 in its center.

IN some embodiments, complete coverage of a target area is generated by advancing gas delivery tube 119C to one or more of its partially- or fully-bent positions, and rotating it through a circular motion. for example, the central region that is a “dead spot” (untreated) in FIGS. 3B and 31C can be reached using the configuration of FIG. 31B, while edges of the treatment area that are missed or only weakly treated by the configuration of 31B receive treatment using the configuration of 31C.

A potential advantage of using a circular scanning motion is that it can easily be controlled by turning a control element located on a proximal side of working channel 115 and in semi-rigid torque communication with the distal end of gas delivery tube 119C. A complete turn (or more than one turn) can be readily judged. Transition between the states of FIGS. 31A-31C may also be readily judged, e.g., by the distance of longitudinal translation of the same or another proximally positioned control element.

It is noted that the area of impingement can also be varied by advancing the whole of plasma delivery tip 3101 closer to or further from surface 11.

Reference is now made to FIGS. 32A-32C, which schematically represent a self-orienting plasma delivery tip 3201 which is actuatable to reorient a plasma outlet aperture 120A through a range of off-axis orientations with respect to a longitudinal axis 13 of the sleeve 117 and/or working channel 115 that delivers it, according to some embodiments of the present disclosure. The example shown uses a discharge electrode assembly 601 connected to power through an electrical conduit 105; alternatively, a circumferential electrode configuration is used.

Gas supply tube 119D is configured to transition from straight to some predefined maximal bend as also described for gas supply tube 119C.

Since outlet aperture 120A is orthogonal to axis 13, plasma plume 10D impinges orthogonally on surface 11 when gas supply tube 119D is minimally extended (FIG. 32A), and axis 13 is itself orthogonal to surface 11. This results in an approximately circular area of plasma impingement 14C that is nearly the same as the area that would be swept out if gas supply tube 119D were rotated around axis 13.

Partial advance of gas supply tube 119D (FIG. 32B) results in an oblique angle of intersection between plasma plume 10D and surface 11, and rotation of the area of static plasma impingement 14D (double-headed arrow 3215) allows sweeping out an approximately circular area; again with a central “dead area” which can be filled in by sweeps made in a less extended configuration.

Full extension of gas supply tube 119D, in this example, leads to plasma plume 10D pointing parallel to (and not impinging on) surface 11. This may be useful, for example, for side-direction of plasma to a surface which extends substantially parallel to longitudinal axis 13. In this connection, it is noted the range of transition angles and rotation angle shown in FIGS. 31A-33C could also be used to treat a substantially cylindrical region while sleeve 117 and/or working channel 115 remain stationary, gaining similar potential advantages for control of coverages as were described for a circular area of surface 11.

Reference is now made to FIGS. 33A-33C, which schematically represent a self-orienting plasma delivery tip 3301 which is actuatable to reorient a plasma outlet aperture 120C through a range of off-axis orientations with respect to a longitudinal axis 13 of the sleeve 117 and/or working channel 115 that delivers it, according to some embodiments of the present disclosure. The configuration in this example is similar to that of FIGS. 32A-32C, except that a hood 114 has been added which acts to deflect plasma plume 10E to an axis oblique to axis 13, even when gas supply tube 119E is almost completely withdrawn into sleeve 117. This may make a larger range of deployed angles useful for plasma delivery (e.g., there is no deployment angle that entirely misses pointing in the direction surface 11, so that there is some impingement area 14E, 14F, 14G shown throughout the range of angulation assumed by gas supply tube 119E). The small dead zone that this example allows around the intersection of axis 13 with surface 11 when rotationally sweeping (double arrow 3315) can be converted by a slight offset or jiggling of working channel 115; or it can be avoided by providing a smaller deflection so that the area of impingement for the mostly-withdrawn configuration includes the intersection of axis 13 and surface 11. FIG. 13B shows another tip configuration which is capable of creating an oblique-angled outlet aperture.

### Plasma Tips Comprising a Plurality of Plasma Generating Sites

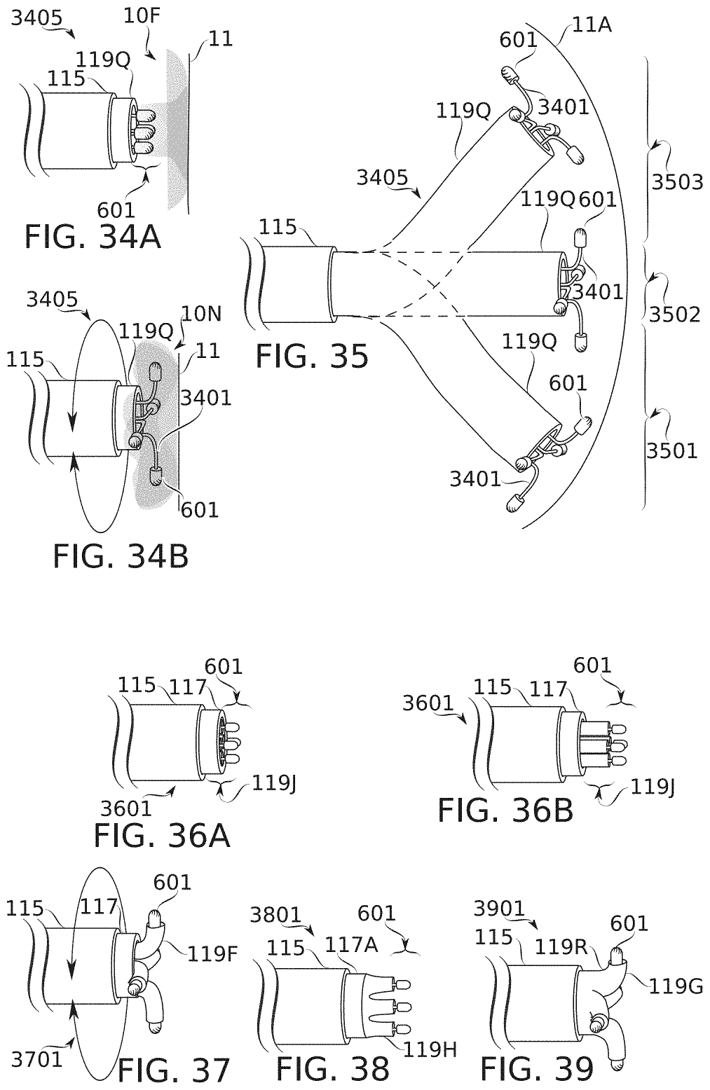

Reference is now made to FIGS. 34A-34B, which schematically illustrate a plasma delivery tip 3405 provided with a plurality of discharge electrode assemblies 601, according to some embodiments of the present disclosure. Reference is also made to FIG. 35, which shown the plasma delivery tip 3405 of FIGS. 34A-34B operated in a steerable configuration, according to some embodiments of the present disclosure.

One of the potential problems with making a plasma plume's cross-section larger is that the electrical field gradient drops off significantly with distance from the discharge electrode, so that plasma generation remains focal around the discharge electrode even when the gas supply tube's cross-section is larger. In the example of FIG. 34A, a plurality of discharge electrode assemblies 601 is provided, around each of which a similar flow of gas can be produced by flow through gas supply lumen 119Q. Each of the discharge electrode assemblies 601 is powered, leading to a plasma plume 10F which is essentially the superposition of several separately generated plasma plumes.

In FIG. 34A, discharge electrode assemblies 601 are partially extended from gas supply tube 119Q; they could additionally or alternatively be retained within gas supply tube 119Q. Their relative spacing is optionally maintained by separating them with a spacer, and/or by joining them together to form one or more multi-headed “candelabra” discharge electrode assemblies. Four discharge electrode assemblies 601 are shown in FIG. 34A; optionally another number is provided, for example, 2, 3, 4, 5, 6, 7 or more discharge electrode assemblies. Optionally, the discharge electrode assemblies 601 are electrically separated from one another, e.g, individually separated, or separated into a plurality of groups. Separated electrodes are optionally powered simultaneously (e.g., from different power supplies), or in a rapid rotation, e.g, using a multiplexing or phase-offset pulsed wave modulation scheme. Time-separated delivery of power to different discharge electrodes potentially helps to reduce non-linear interactions between different plasma generating sites.

Optionally, each discharge electrode assembly 601 is connected to electrical power through a flexible conductive member 3401 which is preconfigured to assume an angulated configuration upon leaving the confinement of gas delivery tube 119Q. In some embodiments, bending of these flexible conductive members 3401 upon their extension displaces the discharge electrode assemblies 601 away from a central axis of gas supply tube 119Q. Optionally, they are displaced into a roughly planar region, with the distance between pairs of most mutually-distant discharge electrode assemblies being larger than the lumenal diameter of gas supply tube 119Q. To put the spread-apart discharge electrode assemblies 601 within the flow of ionization gas, gas supply tube 119Q is brought close enough to a target surface 11 that the confined flow of ionization gas is forced to spread laterally.

Then, discharge electrode assemblies 601 can be advanced into the zone of gas flow to create a plasma plume; for example, plasma plume 10N. Within a sufficiently enclosed lumenal space, redirection of flow is not necessarily required, once ionization gas has been diffused sufficiently therein. Optionally, the discharge electrode assemblies 601 can be rotated together (as indicated by double-headed arrow 3405) to even out plasma contact with surface 11. The rotation may be performed, e.g., by rotation of an electrical conduit to which each of the conductive members 3401 is connected, and/or by rotation of gas supply tube 119Q.

A plasma plume is optionally generated while discharge electrode assemblies 601 are at any suitable intermediate position between those shown in FIGS. 34A-34B.

FIG. 35 shows the superposition of 3 different flexing states of gas supply tube 119Q, which is optionally configured to be bend-controllable; for example as described in relation to FIGS. 3A and/or 14A. This can be useful in particular for treatment of large areas of a curved lumenal wall surface 11A, such as may exist, for example, inside a bladder.

Reference is now made to FIGS. 36A-36B, which schematically illustrate a plasma delivery tip 3601 provided with a plurality of discharge electrode assemblies 601 operable together with a corresponding plurality of individual gas supply tubes 119J, according to some embodiments of the present disclosure.

In FIG. 36A, the individual gas supply tubes 119J remain fully housed within sleeve 117, with discharge electrode assemblies 601 protruding. Optionally, discharge electrode assemblies 601 can be withdrawn completely into sleeve 117 and/or gas supply tubes 119J.

In FIG. 36B, gas supply tubes 119J have been advanced partially out of sleeve 117. This is another way of generating a merged plasma plume, for example as described in relation to plasma plume 10F of FIG. 34A.

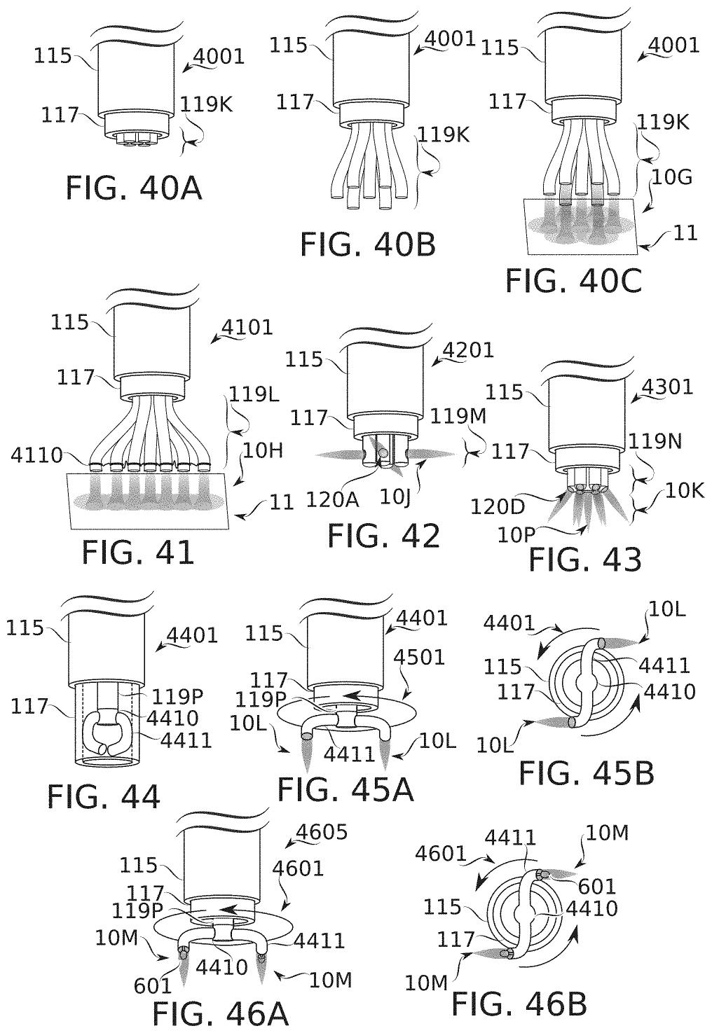

A potential advantage of this more fully individualized embodiment is greater isolation (and, accordingly, independence) of plasma generating sites from each other. Optionally, they can be operated with separate flows of gas and/or from separate electrical power, or joined together. This potentially allows separately tuning of plasma generating parameters for each plasma generating unit (each “unit” comprising one of the gas supply tubes 119J, and one of the discharge electrode assemblies 601. There may also be reduction of cross-talk between plasma generating units, so that plasma generating parameters developed for a single unit are potentially less likely to need adjustment when a plurality of such units are joined together. Other things that can be done with a plurality of separate plasma generating units are described in relation to FIGS. 40A-41; for example, the distal ends of each gas supply tube 119J can be configured to rearrange into a larger (more spread out) pattern as they advance from confinement, and/or a differently shaped (e.g., linear) pattern as they advance from confinement.

Reference is now made to FIGS. 37 and 39, which schematically illustrate a plasma delivery tip 3701 provided with a plurality of discharge electrode assemblies 601, operable together with a corresponding plurality of individual gas supply tubes 119F which spread to radially expanded shape upon advance from confinement, according to some embodiments of the present disclosure. This embodiment may be viewed as a combination of features of FIGS. 34A-35 with features of FIGS. 36A-36B. Instead of spreading out into a common “cloud” of ionization gas, each discharge electrode assembly 601 retains its own gas supply tube 119F, which potentially helps to maintain more stable conditions of plasma generation at each site, as well as better ensuring independent operation of each plasma generating unit. In FIG. 37, each of the gas supply tube 119F is a separate tube, optionally separate all the way back to their proximal ends from which ionization gas is supplied.

As also described in relation to the example of FIG. 34A, the discharge electrode assemblies 601 are optionally ganged together electrically, or electrically separated from one another, e.g, individually separated, or separated into a plurality of groups. Separated electrodes are optionally powered simultaneously (e.g., from different power supplies), or in a rapid rotation, e.g, using a multiplexing or phase-offset pulsed wave modulation scheme. Time-separated delivery of power to different discharge electrodes potentially helps to reduce non-linear interactions between different plasma generating sites.

Plasma delivery tip 3901 of FIG. 39 includes gas supply tubes 119G which branch from a common lumen of a gas delivery tube 117B. A potential advantage of splitting off from a common lumen to the distal end is a reduction in complexity of the device at more proximal locations, compared to tubes that extend the whole length of the device. There may also be, e.g., reduced resistance to gas flow, and/or more room available for electrical interconnections.

Reference is now made to FIG. 38, which schematically illustrates a plasma delivery tip 3801 provided with a plurality of discharge electrode assemblies 601, operable together with a corresponding plurality of individual gas supply tubes 119H which are linearly arranged, and branch from a common lumen of gas supply tube 117A. Three tubes are shown; other numbers of the individual gas supply tubes 119H are optionally provided, for example, 2, 4, 5, 6, 7 or more individual gas supply tubes 119H. A potential advantage of splitting off from a common lumen to the distal end is a reduction in complexity of the device at more proximal locations. There may also be, e.g., reduced resistance to gas flow, and/or more room available for electrical interconnections. As also described for other embodiments described herein, the final separation of gas supply into separate lumens is a potential advantage for providing a larger plasma delivery area using plasma generating parameters determined for a small cross-section device.

Reference is now made to FIGS. 40A-40C, which schematically illustrate a manifold-type plasma delivery tip 4001, according to some embodiments of the present disclosure. Plasma delivery tip 4001 comprises a plurality of gas supply tubes 119K that can be deployed from confinement within a lumen of a sleeve 117 and/or working channel 115. Additionally, (as displayed in FIGS. 40B and 40C) gas supply tubes 119K are configured to bend slightly as they advance from confinement into a wider-area spread (e.g., they are bent when unconfined, and forced together into straighter configurations when confined). Upon generation of plasma (FIG. 40C) the separately generated plasma plumes 10G together impinge upon a larger area of surface 11 than they would from their still-confined positions.

Reference is now made to FIG. 41, which schematically illustrates another manifold-type plasma delivery tip 4101, according to some embodiments of the present disclosure. In this example, the individual gas supply tubes 119L are configured to assume a substantially linear arrangement upon being deployed. This may be at least in part because the gas supply tubes 119L are bent so that they self-arrange into a linear form. Optionally, a truss 4110 is provided which itself has a preferred overall linear shape when unconfined, but enough flexibility and slack to fold upon itself when confined by retraction of gas supply tubes 119L back into sleeve 117 and/or working channel 115.

Reference is now made to FIGS. 42-43, which schematically additional manifold-type plasma delivery tips 4201, 4301, according to some embodiments of the present disclosure. In the example of FIG. 42, at least some of the individual gas supply tubes 119M are provided with lateral outlets 120A, from which plasma plumes 10J are provided. The plasma plumes are oriented in different directions, providing an approximately ring-shaped area of plasma coverage which can be scanned by rotation and/or by longitudinal translation of gas supply tubes 119M. FIG. 43 shows obliquely-angled outlet apertures 120D of gas supply tubes 119N. Angles of outlet apertures can be mixed; for example, the outlet of one of the gas supply tubes 119N is oriented to project plasma plume 10P directly along a longitudinal axis of a distal portion of sleeve 117 and/or working channel 115. This illustrates another way of producing a plasma plume spread wider than the diameter of the lumen used to deliver the plasma delivery tip. As another example of mixed aperture orientations: in some embodiments, another ring of gas supply tubes configured with lateral outlet apertures 120A as shown in FIG. 42, is added around the gas supply tubes 119N of FIG. 43. This may be used to produce an approximately hemispherical pattern of plasma plume generation.

Reference is now made to FIGS. 44-46B, which schematically illustrate embodiments of self-rotating plasma delivery tips 4401, 4605, according to some embodiments of the present disclosure. The jets of ionization gas which are used to generate plasma plumes 10L, 10M can confer a significant amount of thrust. Several liters per minute of ionization gas (e.g., equivalent to about 0.5 to 10 liters of atmospheric pressure gas per minute) may be ejected through apertures having a diameter less than about (for example) 1 mm, 2 mm or 3 mm. In some embodiments, this thrust is put to use by terminating gas delivery tube 119P, in a rotationally-mounted cap 4410, which redirects gas into arms 4411 that are themselves oriented so that gas exiting them induces a component of tangential thrust that causes cap 4410 and arms 4411 to rotate.

In FIG. 44, the arms 4411 are shown collapsed by confinement within sleeve 117. Upon advance from confinement (FIGS. 45A-45B and 46A-46B), arms 4411 extend radially (optionally beyond the diameter of the lumen few sleeve 117), with their outlets oriented so that at least a component of their thrush is directed tangentially. This results in circular motions of arms 4411, as indicated by arrows 4601, 4501. The circular motion causes plasma plumes 10L, 10M to distribute plasma circumferentially.

FIGS. 45A-45B show an example using circumferential electrodes inside the lumens of arms 4411. FIGS. 46A-46B show an example using a discharge electrode assembly that can be positioned within the flow of ionization gas either inside or outside of the lumens of arms 4411. FIGS. 45B and 46A are end-on views of the plasma delivery tip, while FIG. 45A-45B provide lateral views.

While the orientations of the outlets of arms 4411 provide plasma plumes 10L, 10M with a component of tangentially-directed thrust, they are also oriented so that the play of plasma plumes 10L, 10M extends somewhat ahead of (distally to) arms 4411, allowing treatment of a surface positioned also distally ahead of arms 4411. Alternatively, the outlets of arms 4411 are oriented parallel to the longitudinal axis of the distal portion of sleeve 117 and/or working channel 115, or even oriented to direct plasma somewhat proximally. In this and other embodiments herein (e.g., embodiments exemplified by FIGS. 31A-33C), proximally-directed plasma plumes may be provided in order to allow treatment of tissue located adjacent to a body lumen aperture from which the device has been introduced.

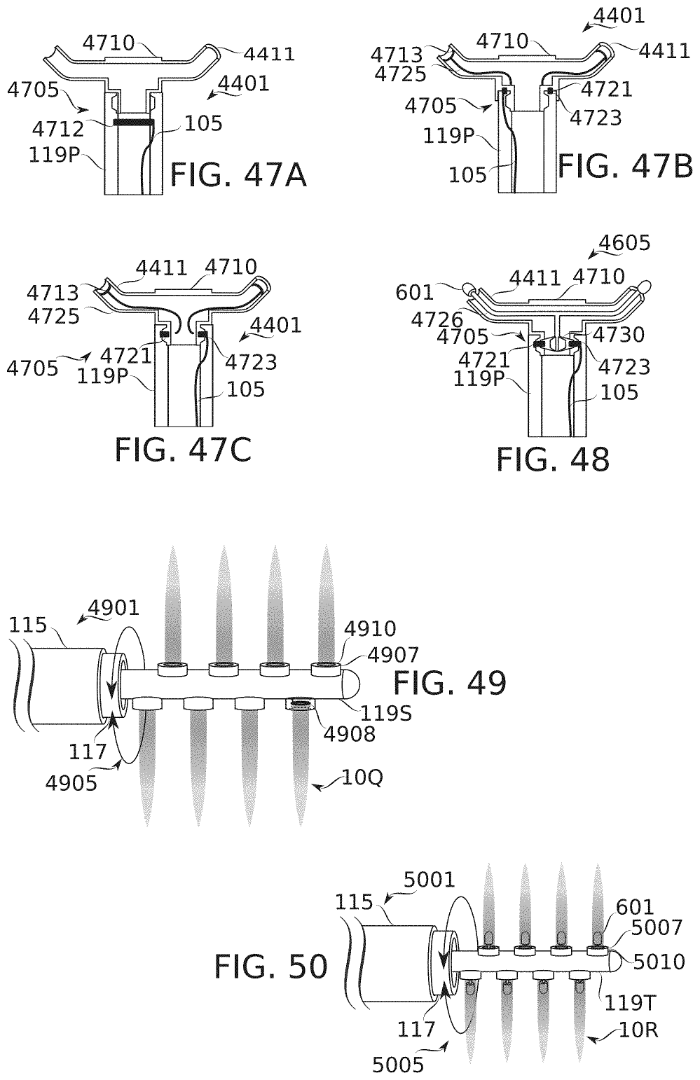

Reference is now made to FIGS. 47A-48, which schematically illustrate alternative embodiments of internal component of self-rotating plasma delivery tips 4401, 4605, according to some embodiments of the present disclosure. These figures focus on methods of transferring (or avoiding transferring) electrical power across the rotating connection 4705 that cap 4710 makes with gas delivery tube 119P.

In FIGS. 47A, electrical conduit 105 leads terminates in discharge electrode 4712, located on a proximal side of the connection 4705. As a result, there is no need to transfer electrical power across a sliding electrical connection. Plasma range may, however, be diminished as a result, since it has to travel further before it reaches the outlets of arms 4411.

The embodiments of FIGS. 47B, 47C, and 48 each use a sliding brush-type arrangement to transfer electrical power across the rotating connection 4705. Electrical conduit 105 terminates in a contact or set of contacts 4723 on the side of gas supply tube 119P, and contact(s) 4723 in turn make sliding electrical contact with contacts 4721. From there, further electrical interconnections 4725, 4726 bring power to discharge electrodes 4713 and/or discharge electrode assemblies 601. Contacts 4721, 4723 may be provided as end-facing elements (e.g., as flat rings in contact along their flat surfaces), for example as shown in FIG. 47B. Additionally or alternatively, contacts 4721, 4723 may be provided as concentric contacts, for example as shown in FIG. 47C. The contacts may also act as bearings. In the arrangement of FIG. 48, a centering element 4730 is provided which stabilizes the mounting of discharge electrode assemblies 601 on their electrical interconnections 4726, as well as itself acting as part of electrical interconnections 4726. Centering element 4730 may be perforated (e.g., constructed of vanes) to permit the flow of gas therethrough.

Reference is now made to FIGS. 49-50, which schematically illustrate plasma delivery tips 4901, 5001 configured with a plurality of longitudinally spaced plasma generation sites 4907, 5007, according to some embodiments of the present disclosure.

The arrangements of FIGS. 49, 50 show further examples of how a plasma generation site which in itself offers relatively limited plasma plume coverage can be duplicated in the design and/or moved (scanned) in operation to expand coverage area. Design duplication allows the design of the plasma delivery tip to rest on individual plasma generating sites as independently designed modules that can be arbitrarily combined. In effect, the modularity “linearizes” otherwise highly non-linear design issues that arise when expanding or otherwise rearranging the plasma generating area of a plasma delivery tip's design. As a result, the arrangement of modules (the plasma generating sites) can be approximated for design purposes as simply additive to each other in their respective areas of coverage.

In some embodiments, a plurality of plasma generation sites 4907, 5007 are spaced along a longitudinal axis of gas supply tube 119S, 119T; each site comprising a module configured to generate its own plasma plume 10Q, 10R. In the examples shown, they plasma generation sites 4907, 5007 are arranged in two alternating rows of plasma generation sites 4907, 5007; with each row's sites being respectively directed in radially opposite directions. Optionally, more than two rows of plasma generation sites 4907, 5007 sites are provided. Optionally, plasma generation sites are arranged otherwise than in longitudinal rows, e.g., helically (e.g., with three or more sites per helical turn). Gas delivery tubes 119S, 119T are rotatable (as indicated by double-headed arrow 5005), either separately from or together with working channel 115 and/or sleeve 117. In some embodiments, the arrangement of plasma generation sites 4907, 5007 is selected so that upon rotation, there is generated effectively continuous coverage of a lumenal wall portion which extends along the longitudinal extent of plasma generation site distribution.

Plasma generation sites 4907 are of the circumferential electrode configuration type, having an at least partially circumferential discharge electrode 4908 which ionizes ionization gas as it flows by through a lumenal substantially interior to the discharge electrode 4908. Plasma generation sites 5007 are of the type that comprises a discharge electrode assembly 601 positioned within the stream of ionization gas—within a lumen of the plasma delivery tip and/or outside of it (as shown).

The plasma generating sites 4907, 5007 shown in FIGS. 49-50 are constructed from tubes 4910, 5010. As shown, the tube lengths are short and ring-like; selected to allow their withdrawal into the lumenal space of sleeve 117 without deformation. Optionally, longer tubes are provided, the tubes comprising a flexible polymer (for example) which collapses upon confinement within sleeve 117, but expanding to point laterally upon release from confinement. Conversely, the tubes may be omitted, and the plasma generating sites implemented simply as apertures within gas delivery tubes 119S, 119T, for example as described in relation to FIGS. 13A-13B, and/or 13D-13F.

In the examples shown, the outlet apertures of plasma generating sites 4907, 5007 are round; optionally, they have another shape, for example as described in relation to FIGS. 17A-19F.

In FIG. 50, the discharge electrode assemblies 601 are shown entirely protruded from their respective tubes 5010, to a distance large enough that they deflect to a collapsed state upon being withdrawn into the confinement of sleeve 117 and/or working channel 115. For example, they may deflect to the side, and/or be mounted on elastic members within lumen 119T that deflect to allow pressing discharge electrode assemblies 601 inward. Optionally, discharge electrode assemblies remain at least partially withdrawn into their respective tubes 5010; optionally enough that they do not need to collapse upon confinement. The discharge electrode assemblies 601 may comprise any of the self-actuating (e.g., thermally self-adjusting) electrode designs described herein. Manually actuated design features are not excluded either, although they may be modified to gang together a plurality of assemblies to a common actuating member.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1-18. (canceled)

19. A plasma delivery tip of a medical-grade plasma generating device, comprising:
a gas delivery lumen having a proximal-to-distal axis, and along which a flow of ionization gas flows distally to an exit aperture of the gas delivery lumen; and
a discharge electrode, located in said lumen, for intraluminal creation of plasma, electrically isolated from the flow of ionization gas by a dielectric barrier layer, and which transmits a high voltage gradient into the flow of ionization gas when attached to a high voltage source, the gradient acting to generate free electrons alongside the discharge electrode and generate a flow of cold plasma by dielectric barrier discharge;
wherein the exit aperture of the gas delivery lumen is oriented to direct a cold plasma plume exiting the gas delivery lumen-, in an oblique direction oriented away from the proximal-to-distal axis,
wherein said plasma delivery tip is suitable for intraluminal delivery using a sheath or working channel and has an outer diameter of less than 7 mm; and
wherein said discharge electrode comprises a central conductor of a coaxial cable comprising said central conductor and an outer conductor, and the central conductor extends distally past said outer conductor and transmits said high voltage gradient.

20. (canceled)

21. The plasma delivery tip of claim 19, wherein said gas lumen comprises a plurality of exit apertures oriented in an oblique direction away from said axis including said exit aperture and wherein said discharge electrode extends circumferentially around said gas lumen.

22. The plasma delivery tip of claim 19, comprising:
at least one gas return channel extending along the gas delivery lumen, through which the ionization gas returns proximally after exiting the gas delivery lumen.

23. The plasma delivery tip of claim 22, wherein the at least one gas return channel extends helically around the gas delivery lumen.

24. The plasma delivery tip of claim 22, wherein the gas return channel is provided with a connector to allow attachment to a source of negative pressure.

25. The plasma delivery tip of claim 22, wherein the gas return channel is open to a pressure lower than a pressure developed negative pressure.

26. The plasma delivery tip of claim 22, wherein the plasma is thermally non-damaging.

27-38. (canceled)

39. The plasma tip of claim 19, comprising:
an outer insulating layer of the coaxial cable surrounding the outer conductor of the coaxial cable, and stripped from a distal portion of the central conductor;
wherein the outer conductor includes an distal extension of a flexible conducting electrical shielding layer from the distal portion of the coaxial cable comprising a stiffened electrical shielding layer; and
an insulator insulating the central conductor with a dielectric barrier layer where it extends distally past the outer conductor.

40. The plasma tip of claim 39, comprising an outer insulating layer added to extend over the stiffened electrical shielding layer.

41. The plasma tip of claim 39, wherein the coaxial cable has an outer diameter of less than 4 mm.

42. (canceled)

43. The plasma delivery tip of claim 19, provided together with the plasma generating device, and operable to generate plasma.
